(19) United States
(12) Patent Application Publication
(10) Pub. No.: US 2026/0308284 A1
(43) Pub. Date: Oct. 8, 2026

(54) SUPPORTED OLEFIN POLYMERIZATION CATALYSTS COMPRISING SUBSTITUTED 2-HYDROXYTHIOPHENE COMPOUNDS

(71) Applicant: Dow Global Technologies LLC, Midland, MI (US)

(72) Inventors: Andrew M. Camelio, Auburn, MI (US); Rhett A. Baillie, Missouri City, TX (US); Matthew L. Krause, Frankenmuth, MI (US)

(21) Appl. No.: 19/475,166

(22) PCT Filed: May 23, 2024
(86) PCT No.: PCT/US2024/030820

§ 371 Date: Oct. 14, 2025

Related U.S. Application Data
(60) Provisional application No. 63/471,823, filed on Jun. 8, 2023.

(51) Int. Cl. C08F 4/76; C07D 409/14; C08F 2/18; C08F 2/34; C08F 210/14

(52) U.S. Cl. CPC C08F 4/76; C07D 409/14; C08F 2/18; C08F 2/34; C08F 210/14; C08F 2500/09

(57) ABSTRACT

Disclosed are a supported catalyst system comprising a substituted 2-hydroxythiophene compound and a support material; and a method of making the supported catalyst system; Also disclosed are a gas phase or slurry phase polymerization process employing the supported catalyst system; and a polyolefin made by the gas phase or slurry phase polymerization process. Also disclosed are the substituted 2-hydroxythiophene compound and a precatalyst comprising the substituted 2-hydroxythiophene compound, a metal atom, and a leaving group. Also disclosed are methods of making the precatalyst and the substituted 2-hydroxythiophene compound.

### FIELD

Olefin polymerization catalysts, materials, and methods.

### INTRODUCTION

Polyolefins are made by generally known methods comprising polymerizing one or more olefin monomers in solution phase catalyzed by homogeneous catalysts, or in slurry phase or gas phase catalyzed by heterogeneous catalysts.

Homogeneous catalysis generally refers to reactions where a soluble catalyst and a reactant it acts upon are in the same phase (same state of matter), and in unrestricted contact. This is almost always liquid phase. The catalyst in pure form and the reactant in pure form at standard temperature and pressure (23° C., 101 kilopascals) may be a solid or a gas, but when the catalyst and reactant are dissolved in the same solution they are both in the liquid phase.

Liquid phase olefin polymerizations mean solution reactions where a homogeneous olefin polymerization catalyst and the reactant—one or more olefin monomers—are dissolved and react in a same hydrocarbon solvent. When the olefin monomer comprises ethylene, the polymerizations are run in hydrocarbon solutions at temperatures from 120° to 250° C., and usually 150° to 190° C., which is above the 115° to 135° C. melting temperature range of polyethylenes.

Homogeneous olefin polymerization catalysts must have at least partial solubility in the hydrocarbon solvent so that, at the relatively low catalyst concentrations and high temperatures used, the entire amount of the catalyst is dissolved in solution. In practice these catalysts are free (unsupported) ligand-metal complex molecules and the hydrocarbon solvent is alkanes or aromatic hydrocarbons.

Structures of free ligand-metal complex molecules may be precisely determined using small molecule structure characterization techniques such as proton-nuclear and carbon-nuclear magnetic resonance (¹H-NMR and/or ¹³C-NMR) spectroscopy or x-ray crystallography. This knowledge enables researchers to make rational design modifications to the homogeneous catalyst to study its structure-activity relationships and structure-product property relationships.

Heterogeneous catalysis generally refers to reactions where an insoluble catalyst and a reactant it acts upon are in different phases (different states of matter). Reaction occurs at interfaces between phases.

Heterogeneous catalysts may be made by a general strategy of heterogenization of homogeneous catalysts or homogeneous precatalysts and activators onto solid supports to yield heterogeneous catalysts in the form of supported catalyst systems. Different supported catalyst systems may require different support materials. For example, Ziegler-Natta catalysts use magnesium chloride and supported metallocene catalysts use silica. Supported catalyst structures cannot be precisely determined.

In gas phase/solid phase olefin polymerizations, called gas phase polymerizations, the supported catalyst system (a heterogeneous olefin polymerization catalyst) is in a solid phase and the reactant—one or more olefin monomers—is in a gas or vapor phase. Reaction occurs at solid phase/gas phase interfaces.

In liquid phase/solid phase olefin polymerizations, called slurry phase polymerizations, the supported catalyst system (a heterogeneous olefin polymerization catalyst) is in a solid phase and the reactant—one or more olefin monomers—is dissolved in a hydrocarbon solvent to give a solution that constitutes the liquid phase. Reaction occurs at solid phase/liquid phase interfaces.

When the olefin monomer comprises ethylene, the gas phase and slurry phase polymerizations are run at from 75° to 120° C., below melting temperatures of most polyethylenes.

For these and other reasons, supported catalyst systems produce significantly different performance results and product properties than those of their counterpart homogeneous olefin polymerization catalysts. Thus, homogeneous olefin polymerization catalysis/solution phase polymerizations are not predictive of heterogeneous olefin polymerization catalysis/gas or slurry phase polymerizations.

### SUMMARY

We claim a supported catalyst system comprising a substituted 2-hydroxythiophene compound and a support material; and a method of making the supported catalyst system. Also claimed are a gas phase or slurry phase polymerization process employing the supported catalyst system; and a polyolefin made by the gas phase or slurry phase polymerization process. Also claimed are the substituted 2-hydroxythiophene compound and a precatalyst comprising the substituted 2-hydroxythiophene compound, a metal atom, and a leaving group. Also claimed are methods of making the precatalyst and the substituted 2-hydroxythiophene compound.

### BRIEF DESCRIPTION OF THE DRAWINGS

FIG. 1 shows Scheme 1 directed to a synthesis of an intermediate compound.

FIG. 2 shows Scheme 2 directed to a synthesis of a substituted 2-hydroxythiophene compound (I).

FIG. 3 shows Scheme 3 directed to a synthesis of a precatalyst of formula (II).

FIG. 4 shows Scheme 4 directed to the making of a spray-dried supported catalyst system (III) or a conventionally-dried supported catalyst system (IV).

FIG. 5 shows Mark-Houwink plots of ethylene/1-hexene copolymers produced from spray-dried supported catalyst system 1 (SCS 1) and a comparative polyethylene homopolymer.

FIG. 6 shows Mark-Houwink plots of ethylene/1-hexene copolymers produced from SCS 1 and a comparative polyethylene homopolymer.

FIG. 7 shows Mark-Houwink plots of ethylene/1-hexene copolymers produced from spray-dried supported catalyst system 3 (SCS 3) and a comparative polyethylene homopolymer.

FIG. 8 shows Mark-Houwink plots of ethylene/1-hexene copolymers produced from SCS 3 and a comparative polyethylene homopolymer.

FIG. 9 shows Mark-Houwink plots of ethylene/1-hexene copolymers produced from spray-dried supported catalyst system 5 (SCS 5) and a comparative polyethylene homopolymer.

FIG. 10 shows Mark-Houwink plots of ethylene/1-hexene copolymers produced from SCS 5 and a comparative polyethylene homopolymer.

FIG. 11 shows Mark-Houwink plots of ethylene/1-hexene copolymers produced from spray-dried supported catalyst system 7 (SCS 7) and a comparative polyethylene homopolymer.

FIG. 12 shows Mark-Houwink plots of ethylene/1-hexene copolymers produced from spray-dried supported catalyst system 9 (SCS 9) and a comparative polyethylene homopolymer.

FIG. 13 shows Mark-Houwink plots of ethylene/1-hexene copolymers produced from spray-dried supported catalyst system 11 (SCS 11) and a comparative polyethylene homopolymer.

FIG. 14 shows Mark-Houwink plots of ethylene/1-hexene copolymers produced from SCS 11 and a comparative polyethylene homopolymer.

FIG. 15 shows Mark-Houwink plots of ethylene/1-hexene copolymers produced from SCS 13 and a comparative polyethylene homopolymer.

FIG. 16 shows Scheme 4 directed to a synthesis of a bis(iodophenoxy)propylene compound.

FIG. 17 has pictorial illustrations of representative chain structures of LLDPE, LDPE, and HDPE.

### DETAILED DESCRIPTION

We claim a supported catalyst system comprising a substituted 2-hydroxythiophene compound and a support material; and a method of making the supported catalyst system. Also claimed are a gas phase or slurry phase polymerization process employing the supported catalyst system; and a polyolefin made by the gas phase or slurry phase polymerization process. Also claimed are the substituted 2-hydroxythiophene compound and a precatalyst comprising the substituted 2-hydroxythiophene compound, a metal atom, and a leaving group. Also claimed are methods of making the precatalyst and the substituted 2-hydroxythiophene compound.

### Synthesis of the Supported Catalyst System

There are two general strategies for making the supported catalyst system comprising a substituted 2-hydroxythiophene compound. A first strategy comprises heterogenizing a homogeneous olefin polymerization precatalyst comprising the substituted 2-hydroxythiophene compound (“homogenous precatalyst”). A second strategy comprises heterogenizing a homogeneous olefin polymerization catalyst comprising the substituted 2-hydroxythiophene compound (“homogeneous catalyst”).

There are two main contacting routes for the first heterogenization strategy comprising heterogenizing the homogeneous precatalyst. A first route comprises contacting a solution of the homogeneous precatalyst in a hydrocarbon solvent onto a solid support that has been pretreated with an activator (also called a cocatalyst), yielding the supported catalyst system. An example of the solid support that has been pretreated with an activator is the spray-dried methylaluminoxane/hydrophobic fumed silica (“SMAO”), which may be used as a convenient way of making an embodiment of the supported catalyst system for use in slurry phase polymerization. A second route comprises contacting an activator with solid support that has been pretreated with the homogeneous precatalyst, yielding the supported catalyst system.

A third contacting route is used for the second heterogenization strategy comprising heterogenizing the homogeneous catalyst. This third route comprises contacting a solution of the homogeneous precatalyst in a hydrocarbon solvent with an activator to give the homogeneous catalyst dissolved in hydrocarbon solvent, and then contacting the solution with the solid support, yielding the supported catalyst system.

The second and third contacting routes are disfavored for use with solid supports that give side reactions with either the homogeneous precatalyst or homogeneous catalyst. The first heterogenization strategy comprising the first contacting route usually does not suffer from this potential problem.

The heterogenization strategies and contacting routes independently make the supported catalyst system as a suspension of solid particles thereof in a liquid consisting essentially of the hydrocarbon solvent and any hydrocarbon-soluble compounds. The hydrocarbon-soluble compounds may include unreacted activator (e.g., methyl aluminoxane or triethylaluminum) and/or by-products and side products from the heterogenization reaction and/or the activation reaction.

In some embodiments the suspension, including any hydrocarbon-soluble compounds, from the contacting route is fed into a gas phase or slurry phase polymerization reactor to polymerize olefin monomer. Prior to or during the feeding step the suspension may or may not be stored for a period of time in a storage tank and/or may or may not be diluted with additional hydrocarbon solvent, which may be the same as or different than the hydrocarbon solvent used in the contacting route.

In other embodiments the suspension from the contacting route is not fed into a gas phase or slurry phase polymerization reactor. Instead the contacting route used to make the supported catalyst system is followed by a separating step, which is performed prior to any feeding of the supported catalyst system into a gas phase or slurry phase polymerization reactor. In such embodiments the separating step comprises physically removing the supported catalyst system solids from the liquid portion of the suspension obtained from the contacting route, or vice versa physically removing the liquid portion from the supported catalyst system solids.

In some embodiments the separating step comprises a filtering step, a decanting step, or an evaporating step. The separating step may also comprise a combination of any two or more separating steps.

The filtering step may comprise contacting the suspension with a filter to yield a filtrate consisting of the liquid portion and a filtercake consisting of the supported catalyst system (solids). The filtercake may be washed with fresh hydrocarbon solvent and/or dried.

The decanting step may comprise pouring off or suctioning off the liquid portion of the suspension, yielding a decanted or suctioned liquid and the supported catalyst system (solids) as a “paste” consisting of the supported catalyst system (solids) and a small remainder of undecanted or unsuctioned liquid. The paste may be used as is in a polymerization or dried or slurried with fresh hydrocarbon solvent.

The drying step may comprise removing volatile constituents from the suspension, yielding the supported catalyst system (solids) as a dry powder. The volatile constituents may include any volatile components of the hydrocarbon-soluble compounds mentioned earlier, such as any volatile unreacted activator and/or volatile by-products and side products from the heterogenization reaction and/or the activation reaction. The drying step may comprise slowly evaporating volatile constituents from the suspension, which is slowly concentrated, yielding a “conventionally-dried” embodiment of the dry powder of the supported catalyst system. Alternatively, the drying step may comprise spray-drying the suspension so as to rapidly remove (flash off) volatile constituents from the suspension, yielding a “spray-dried” embodiment of the dry powder of the supported catalyst system.

The combination of any two or more separating steps may comprise, for example, the decanting step followed by the evaporating step or two sequential decanting steps.

The spray-dried supported catalyst system embodiments can have higher catalyst efficiencies, higher catalyst productivities, faster light-offs, and can produce polyethylene polymers having different properties in gas phase polymerizations than the conventionally-dried embodiments of the supported catalyst system have in gas phase polymerizations. Thus, the spray-dried supported catalyst system embodiments may be preferred over the conventionally-dried supported catalyst system embodiments for gas phase polymerizations. Nonetheless, the conventionally-dried supported catalyst system embodiments are also completely useful and effective for gas phase polymerizations.

There may be little or no difference between the spray-dried supported catalyst system embodiments and the conventionally-dried supported catalyst system embodiments in slurry phase polymerizations, or there may be significant differences. However, any given spray-dried supported catalyst system, or any given conventionally-dried supported catalyst system, may perform quite differently in gas phase polymerizations than in slurry phase polymerizations.

All dry powder embodiments of the supported catalyst system are versatile for gas phase and slurry phase polymerizations because they can be fed as a dry powder, or suspended in alkanes or mineral oil and the resulting suspension fed, into gas phase or slurry phase olefin polymerization reactors. Catalyst feeders for both methods are commercially available.

The supported catalyst system, no matter its physical constitution (e.g., as dry powder or as a powder suspended in hydrocarbon solvent) is useful for catalyzing gas phase or a slurry phase olefin polymerization of one or more olefin monomers to make polyolefins such as polyethylene polymers.

### Technical Advantages

Homogeneous olefin polymerization catalysis in a solution phase reaction with the counterpart homogeneous catalyst comprising a substituted 2-hydroxythiophene compound is quite different than heterogeneous olefin polymerization catalysis in a gas phase or slurry phase reaction with the supported catalyst system. The former is not predictive of the latter and polyolefin products obtained from the latter are different than polyolefin products obtained from the former in various properties such as polymer weight average molecular weight, melt rheology, and branching.

The supported catalyst system and polyolefins made by the supported catalyst system via gas phase or slurry phase olefin polymerization have technical advantages relative to the counterpart homogeneous catalyst and polyolefin made by the homogeneous catalyst via solution phase olefin polymerization. These technical advantages include one or more of increased catalyst efficiencies or productivities, improved behavior in gas phase reactors, and differing product polyolefin polymer properties and morphologies. These technical advantages result in different types of unpredictable results: performance differences between the inventive supported catalyst system and a comparative homogeneous olefin polymerization catalyst; property differences between an inventive polyolefin and a comparative polyolefin; performance differences between different embodiments of the inventive supported catalyst system; and property differences between different embodiments of the inventive polyolefin. Further, the spray-dried embodiments of the supported catalyst system tend to have more technical advantages than the conventionally dried embodiments of the supported catalyst system.

Without being bound by theory, we believe that the technical advantages of the inventive supported catalyst system, comprising the substituted 2-hydroxythiophene compound, in gas phase or slurry phase polymerizations are functions of one or more of the following factors: (a) effects of the solid support, (b) performance differences between different embodiments of the supported catalyst system depending on if the embodiment was made via heterogenization according to the first, second, or third route, (c) the different effects of the conventional drying method versus the spray-drying method used to make dried powders of the supported catalyst system, (d) the effects of differences in process conditions between solution phase versus gas phase or slurry phase, (e) performance differences between different embodiments of the supported catalyst system in their gas phase reactor behavior, or (f) any combination of two or more of effects (a) to (e).

The (a) effects of the solid support may vary with the olefin—polar group-containing olefin monomers being especially sensitive to the solid support, and with the solid support's surface chemistry, which in turn is affected by whether or not it has been pretreated with a hydrophobing agent and the hydrophobing agent used.

The (b) effects of the route used to heterogenize the homogeneous olefin polymerization catalyst to make different embodiments of the supported catalyst system may vary depending upon if the first, second, or third route is used, or a different route.

The (c) effects of the drying method used to make the dry powder of the supported catalyst system may vary depending on whether or not the drying step is employed and the type of the drying step, e.g., conventional drying versus spray-drying. In some embodiments the inventive method comprises spray-drying.

The differences in (d) process conditions comprise reaction temperature differences. Solution phase polymerizations of ethylene are run at temperatures from 140° to 250° C., typically 150° to 190° C., whereas gas phase and slurry phase polymerizations of ethylene are run at lower temperatures, from 70° to 120° C., usually from 750 to 115° C. These temperature differences affect catalyst efficiencies and productivities and polyethylene properties such as molecular weights (e.g., weight-average molecular weights), which may vary significantly at different reaction temperatures. For example, these relationship differences are at least in part due to differences in reaction rates for competing reactions comprising polyethylene chain propagation versus polyethylene chain termination in solution phase at 150° to 190° C. versus these competing reaction rates in gas phase or slurry phase at 75° to 115° C. For example, all other things being equal, if the ratio of the chain propagation reaction rate to the chain termination reaction rate increases as reaction temperature decreases, the catalyst efficiencies and productivities and molecular weights will increase (improve), whereas if the ratio decreases, these relationships decrease (worsen).

The performance differences between different embodiments of the supported catalyst system in (e) gas phase reactor behavior comprise kinetics of the supported catalyst system on its light-off kinetics for freshly fed catalyst, maximum temperature reached after feed (temperature will increase due to exothermic nature of olefin polymerization reactions), or the amount of ethylene uptake per unit weight of catalyst.

The (f) combinations of two or more of factors (a) to (e) are a further technical advantage of the inventive heterogeneous olefin polymerization catalyst comprising the substituted 2-hydroxythiophene compound and the polyolefin made via gas phase or slurry phase olefin polymerization catalyzed thereby.

In some embodiments the supported catalyst system comprising the substituted 2-hydroxythiophene compound has an improved activity in a gas phase and slurry phase polymerization reaction relative to that activity of its counterpart homogeneous olefin polymerization catalyst. For example, the improved activity may be an increased catalyst efficiency and/or an increased catalyst productivity. In some embodiments the supported catalyst system also makes a polyethylene product with one or more improved properties relative to those properties of a polyethylene product made by its counterpart homogeneous olefin polymerization catalyst in solution phase polymerization. For example, the improved property may be an increased weight-average molecular weight (Mw); an increased content of ultra-high molecular weight (“UHMW”) constituents, e.g., Mw greater than 1,000,000 grams per mole (g/mol); a z-average molecular weight greater than 2,000,000 grams per mole; a broader molecular weight distribution (MWD) or polydispersity index (PDI), e.g., Mw/Mn; an increased long chain branching (LCB) content; or a combination of any two or more thereof.

### Additional Embodiments

Another embodiment is a substituted 2-hydroxythiophene compound of formula (I):

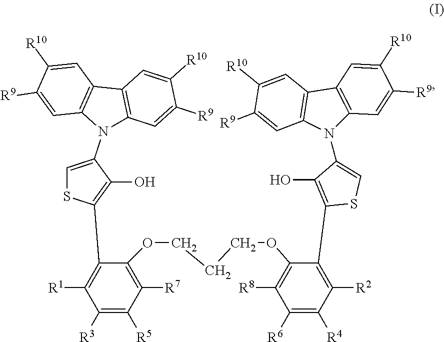

or a Group 1 or Group 2 metal salt thereof. R¹, R², R³, R⁴, R⁵, R⁶, R⁷, R⁸, R⁹, and R¹⁰ are defined below.

Another embodiment is a precatalyst of formula (II):

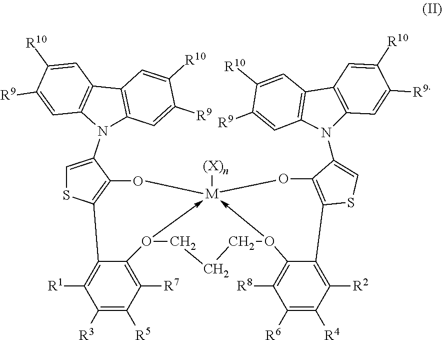

R¹, R², R³, R⁴, R⁵, R⁶, R⁷, R⁸, R⁹, R¹⁰, M, X, and subscript n are defined below.

Another embodiment is a catalyst made by contacting the precatalyst of formula (II) with an activator. The catalyst is useful for polymerizing one or more olefin monomers.

Another embodiment is a supported catalyst system comprising the precatalyst of formula (II), a support material, and an activator.

Another embodiment is a method of making the supported catalyst system, the method comprising step (a) or comprising steps (b) and (c): (a) spray drying a mixture of an inert hydrocarbon solvent, the precatalyst of formula (II), the support material, and the activator to make the supported catalyst system; or (b) spray drying a mixture of an inert hydrocarbon solvent, the support material and the activator to make a spray-dried supported activator, and (c) mixing the precatalyst of formula (II) with the spray-dried supported activator and an inert hydrocarbon solvent to make the supported catalyst system.

Another embodiment is a method of polymerizing an olefin monomer, the method comprising contacting the olefin monomer with the supported catalyst system, thereby making a polyolefin. The method may comprise a gas phase polymerization in a gas phase reactor under gas phase conditions or a slurry phase polymerization in a slurry phase reactor under slurry phase conditions.

Another embodiment is the polyolefin made by the method of polymerizing.

Independently in the formulas (I) and (II), R¹ and R² independently are H or a halogen. In some embodiments R¹ and R² are different, or R¹ and R² are identical. In some embodiments R¹ and R² are H. In other embodiments R¹ and R² are F.

Independently in the formulas (I) and (II), R³ and R⁴ independently are H, a halogen, a (C₁-C₁₅)hydrocarbyl, a (C₁-C₁₀)alkoxy, or a Si((C₁-C₁₀)alkyl)₃. In some embodiments R³ and R⁴ are different, or R³ and R⁴ are identical. In some embodiments R³ and R⁴ are a halogen, a (C₁-C₁₅)hydrocarbyl, a (C₁-C₁₀)alkoxy, or a Si((C₁-C₁₀)alkyl)₃. In some embodiments R³ and R⁴ are H. In other embodiments R³ and R⁴ are F. In some embodiments R³ and R⁴ are a (C₁-C₁₅)hydrocarbyl. In other embodiments R³ and R⁴ are a (C₁-C₁₀)alkoxy. In other embodiments R³ and R⁴ are a Si((C₁-C₁₀)alkyl)₃. In some embodiments R³ and R⁴ are identical and are both H, or both F, or both —C(CH₃)₃, or both —C(CH₂CH₃)₃, or both —C(CH₃)₂CH₂C(CH₃)₃, or both —OCH₃, or both —O(CH₂)₂C(CH₃)₃, or both —O(CH₂)₇CH₃, or both 4-i(tert-butyl)phenyl, or both 1,3-di(tert-butyl)phenyl, or both —Si(CH₃)₂(CH₂)₇CH₃. In some embodiments each (C₁-C₁₅)hydrocarbyl independently is a (C₁-C₁₅)alkyl, a (C₁-C₅)alkyl, a (C₆-C₁₀)alkyl, a (C₆-C₁₅)aryl (e.g., phenyl or naphthyl), a (C₇-C₁₅)aralkyl (e.g., benzyl, 2-phenylethyl, or 1-phenylprop-1-yl), or a (C₇-C₁₅)alkaryl (e.g., 4-methylphenyl or 2,6-diisopropylphenyl).

Independently in the formulas (I) and (II), R⁵ and R⁶ independently are H or a halogen. In some embodiments R⁵ and R⁶ are different, or R⁵ and R⁶ are identical. In some embodiments R⁵ and R⁶ are H. In some embodiments R⁵ and R⁶ are F.

Independently in the formulas (I) and (II), R⁷ and R⁸ independently are H or a halogen. In some embodiments R⁷ and R⁸ are different, or R⁷ and R⁸ are identical. In some embodiments R⁷ and R⁸ are H. In some embodiments R⁷ and R⁸ are F.

In some embodiments R¹, R², R³, R⁴, R⁵, R⁶, R⁷, and R⁸ are H.

In some embodiments R³, R⁴, R⁵, and R⁶ are F and R¹, R², R⁷, and R⁸ are H. In some embodiments R¹, R², R³, R⁴, R⁵, and R⁶ are F and R⁷ and R⁸ are H. In some embodiments R³, R⁴, R⁵, R⁶, R⁷, and R⁸ are F and R¹ and R² are H.

In some embodiments R¹, R², R⁵, R⁶, R⁷, and R⁸ are H and R³ and R⁴ are as defined above with the proviso that R³ and R⁴ are not H.

Independently in the formulas (I) and (II), in some embodiments each R⁹ is H and each R¹⁰ is a (C₁-C₁₅)hydrocarbyl, alternatively a (C₁-C₁₀)alkyl or a (C₁-C₅)alkyl, phenyl, or substituted phenyl; or each R¹⁰ is H and each R⁹ is a (C₁-C₁₅)hydrocarbyl, alternatively a (C₁-C₁₀)alkyl or a (C₁-C₅)alkyl, phenyl, or substituted phenyl. In other embodiments each R⁹ is H and each R¹⁰ is a —Si((C₁-C₁₀)alkyl)₃, (C₁₀-C₁₈)aryl, or substituted (C₁₀-C₁₈)aryl; or each R¹⁰ is H and each R⁹ is a —Si((C₁-C₁₀)alkyl)₃, (C₁₀-C₁₈)aryl, or substituted (C₁₀-C₁₈)aryl. Each substituted phenyl has from 1 to 3 substituent groups independently selected from F, (C₁-C₁₀)alkyl, and (C₁-C₁₀)alkoxy; or F and (C₁-C₁₀)alkoxy; or (C₁-C₁₀)alkyl. In some embodiments each R⁹ is H and each R¹⁰ is tertiary-butyl, 4-tert-butylphenyl, 4-triethylmethylphenyl, 3,5-dimethylphenyl, 3,5-di-tert-butylphenyl, or 3,5-difluoro-4-octyloxyphenyl. In other embodiments each R¹⁰ is H and each R⁹ is 3,5-di-tert-butylphenyl.

In some embodiments R¹ and R² are different, or R¹ and R² are identical; or R¹ and R² are H; or R¹ and R² are F; or R³ and R⁴ are different, or R³ and R⁴ are identical, or R³ and R⁴ are a halogen, a (C₁-C₁₅)hydrocarbyl, a (C₁-C₁₀)alkoxy, or a Si((C₁-C₁₀)alkyl)₃, or R³ and R⁴ are a (C₁-C₁₀)alkyl or a (C₁-C₁₀)alkoxy; or R⁵ and R⁶ are different, or R⁵ and R⁶ are identical, or R⁵ and R⁶ are H, or R⁵ and R⁶ are F; or R⁷ and R⁸ are different, or R⁷ and R⁸ are identical, or R⁷ and R⁸ are H, or R⁷ and R⁸ are F; or each R⁹ is H and each R¹⁰ is tertiary-butyl, 4-tert-butylphenyl, 4-triethylmethylphenyl, 3,5-dimethylphenyl, 3,5-di-tert-butylphenyl, or 3,5-difluoro-4-octyloxyphenyl; or each R¹⁰ is H and each R⁹ is 3,5-di-tert-butylphenyl.

Independently in formula (II), M is Ti, Hf, or Zr. In some embodiments M is Hf or Zr, or M is Ti, or M is Hf, or M is Zr.

Independently in formula (II), subscript n is 1, 2, or 3. In some embodiments subscript n 1 or 2, or n is 2 or 3, or n is 1, or n is 2, or n is 3.

Independently in formula (II), each X independently is a leaving group, at least one of which is displaceable when precatalyst (II) is contacted with an activator. In some embodiments each X independently is selected from a monodentate ligand independently chosen from a hydrogen atom, a (C₁-C₅₀)hydrocarbyl, a (C₁-C₅₀)heterohydrocarbyl, a (C₁-C₅₀)organoheteryl, a halogen atom, a dialkylamino, or a dialkyl carbamate. Each heteroatom in a heterohydrocarbyl or organoheteryl may be O, N, S, Si, or P. In some embodiments each heteroatom is O, N, or Si, or each heteroatom is Si. In some embodiments each X a halogen, a (C₁-C₈)alkyl group, a Si((C₁-C₈)alkyl)₃ group, a CH₂Si((C₁-C₁₀)alkyl)₃ group, or benzyl. In some embodiments each X is benzyl or each X is Cl and subscript n is 2; or each X is benzyl and subscript n is 2.

The subscript n and X are chosen so that the precatalyst of formula (II) is overall (i.e., formally) charge-neutral.

Independently in formula (I) and (II), in some embodiments R¹ and R² are identical, R³ and R⁴ are identical, R⁵ and R⁶ are identical, R⁷ and R⁸ are identical, each R⁹ is identical, each R¹⁰ is identical, each R¹¹ is identical, and each R¹² is identical. In some such embodiments R¹ and R² are H; R³ and R⁴ are a halogen, a (C₁-C₁₅)hydrocarbyl, a (C₁-C₁₀)alkoxy, or a Si((C₁-C₁₀)alkyl)₃; R⁵ and R⁶ are H; and R⁷ and R⁸ are H. In some such embodiments R³ and R⁴ are as defined earlier. In some embodiments each X is identical. In some of these embodiments M is Hf. In some of these embodiments M is Zr.

Independently in formula (I) and (II), in some embodiments R¹ and R² are H, R³ and R⁴ are each a (C₁-C₁₅)alkyl or a (C₁-C₁₀)alkoxy, R⁵ and R⁶ are H, R⁷ and R⁸ are H, each R⁹ is H, and each R¹⁰ is the same and is tertiary-butyl, 4-tert-butylphenyl, 4-triethylmethylphenyl, 3,5-dimethylphenyl, or 3,5-di-tert-butylphenyl. In other embodiments R¹ and R² are H, R³ and R⁴ are a (C₇-C₉)alkyl or a (C₇-C₉)alkoxy, R⁵ and R⁶ are H, R⁷ and R⁸ are H, each R⁹ is H, and each R¹⁰ is tertiary-butyl. In some embodiments each R³ and R⁴ is (CH₃)₃CCH₂C(CH₃)₂— or CH₃(CH₂)₇O—.

Independently in formula (I) and (II), in some embodiments R¹ and R² are F, or R⁵ and R⁶ are F, or R⁷ and R⁸ are F, or at least four thereof are F.

Independently in formula (I) and (II), in some embodiments R¹ and R² are the same, R³ and R⁴ are the same, R⁵ and R⁶ are the same, R⁷ and R⁸ are the same, each R⁹ is the same, each R¹⁰ is the same, and, in formula (II), each X is the same.

In some embodiments the substituted 2-hydroxythiophene compound of formula (I) is free of a Group 1 or Group 2 metal, i.e., has the structure drawn for formula (I). In other embodiments the substituted 2-hydroxythiophene compound is the Group 1 or Group 2 metal salt thereof.

The Group 1 or Group 2 metal salt may be made by replacing the hydrogen atom of one of the hydroxyl groups or replacing each of the hydrogen atoms of both hydroxyl groups, of the substituted 2-hydroxythiophene compound in formula (I) with a Group 1 or Group 2 metal atom. This may be done by reacting the compound of formula (I) with a Group 1 or Group 2 metal reactant. The Group 1 or Group 2 metal reactant may be a Group 1 or Group 2 metal hydroxide, a Group 1 or Group 2 metal hydride, a Group 1 or Group 2 metal alkoxide, or an alkyl Group 1 or Group 2 metal. In some embodiments the Group 1 or Group 2 metal atom or metal atoms independently is Li, Na, K, Ca, or Mg. The quantity of Group 1 or Group 2 metal reactant is chosen so that the Group 1 or Group 2 metal salt of the precatalyst of formula (I) is overall (i.e., formally) charge-neutral.

In some embodiments the substituted 2-hydroxythiophene compound of formula (I) is selected from the group consisting of compounds 1 to 6 in TABLE 1:

TABLE 1

| Cmpd | R¹/R² | R³/R⁴ | R⁵/R⁶ | R⁷/R⁸ | | |
|---|---|---|---|---|---|---|
| No. | each is | each is | each is | each is | R⁹ | R¹⁰ |
| 1 | H | t-Octyl | H | H | H | t-Bu |
| 2 | H | OctylO | H | H | H | t-Bu |
| 3 | H | Et₃C— | H | H | H | t-Bu |
| 4 | H | 3,5-dtBP | H | H | H | t-Bu |
| 5 | H | C₈Me₂Si | H | H | H | t-Bu |
| 6 | H | 4-Et₃CPh | H | H | H | t-Bu |

wherein “Cmpd No.” is compound number, t-Bu is tertiary-butyl; t-Octyl is (CH₃)₃CCH₂C(CH₃)₂—; OctylO is CH₃(CH₂)₇O—; 3,5-dtBP is 3,5-di-tert-butylphenyl or 3,5-di(t-Bu)-Ph; C₈Me₂Si is CH₃(CH₂)₇(CH₃)₂Si- (octyl-dimethyl-silyl); and 4-Et₃CPh is 4-triethylmethylphenyl (i.e., 4-[(CH₃CH₂)₃C]-C₆H₄—); and OctylOPh is 4-octyloxyphenyl.

In some embodiments is the precatalyst of formula (II) selected from the group consisting of precatalyst numbers 1 to 13 in TABLE 2:

TABLE 2

| Precatalyst | Make from Formula | | X | |
|---|---|---|---|---|
| No. | (I) Compound No. | M | each is | n |
| 1 | 1 | Zr | Benzyl | 2 |
| 2 | 1 | Hf | Benzyl | 2 |
| 3 | 2 | Zr | Benzyl | 2 |
| 4 | 2 | Hf | Benzyl | 2 |
| 5 | 3 | Zr | Benzyl | 2 |
| 6 | 3 | Hf | Benzyl | 2 |
| 7 | 4 | Zr | Benzyl | 2 |
| 8 | 4 | Hf | Benzyl | 2 |
| 9 | 5 | Zr | Benzyl | 2 |
| 10 | 5 | Hf | Benzyl | 2 |
| 11 | 6 | Zr | Benzyl | 2 |
| 12 | 6 | Hf | Benzyl | 2 |
| 13 | 2 | Zr | Cl | 2 |

In some embodiments is the supported catalyst system selected from the group consisting of spray-dried supported catalyst system numbers SOS 1 to SOS 13 and undried supported catalyst system numbers SCS 14 to SCS 16 in TABLE 3:

TABLE 3

| | Make from Formula | Support | | Catalyst Drying |
|---|---|---|---|---|
| SCS No. | (II) Precatalyst No. | Material | Activator | Method |
| SCS 1 | 1 | HPFS1 | MAO | Spray |
| SCS 2 | 2 | HPFS1 | MAO | Spray |
| SCS 3 | 3 | HPFS1 | MAO | Spray |
| SCS 4 | 4 | HPFS1 | MAO | Spray |
| SCS 5 | 5 | HPFS1 | MAO | Spray |
| SCS 6 | 6 | HPFS1 | MAO | Spray |
| SCS 7 | 7 | HPFS1 | MAO | Spray |
| SCS 8 | 8 | HPFS1 | MAO | Spray |
| SCS 9 | 9 | HPFS1 | MAO | Spray |
| SCS 10 | 10 | HPFS1 | MAO | Spray |
| SCS 11 | 11 | HPFS1 | MAO | Spray |
| SCS 12 | 12 | HPFS1 | MAO | Spray |
| SCS 13 | 13 | HPFS1 | MAO | Spray |

| | | | |
|---|---|---|---|
| SCS 14 | 1 | SMAO | None (undried) |
| SCS 15 | 2 | SMAO | None (undried) |
| SCS 16 | 3 | SMAO | None (undried) |

wherein “~HPFS1” is a hydrophobic fumed silica made from untreated fumed silica and a hydrophobing agent that is dichlorodimethylsilane; and “MAO” is methylaluminoxane; and wherein “SMAO” is spray dried methylaluminoxane/HPFS1, wherein HPFS1 is made from untreated fumed silica and a hydrophobing agent that is dichlorodimethylsilane.

In some embodiments the supported catalyst system is that which has been shown to make by gas phase polymerization an ethylene/1-hexene copolymer having a weight-average molecular weight greater than 1,000,000 grams per mole and/or a z-average molecular weight greater than 2,000,000 grams per mole.

In some embodiments the substituted 2-hydroxythiophene compound of formula (I) is selected from Cmpd. nos. 1 to 6; or from any five of Cmpd. Nos. 1 to 6; or from Cmpd. nos. 1 and 2; or from Cmpd. nos. 3 and 4; or from Cmpd. nos. 5 and 6.

In some embodiments the precatalyst of formula (II) is selected from Precat. nos. 1 to 12; or from any eleven of Precat. Nos. 1 to 13; or from Precat. nos. 1 to 9, 11, and 12; or from Precat. nos. 1 and 2; or from Precat. nos. 3 and 4; or from Precat. nos. 5 and 6; or from Precat. nos. 7 and 8; or from Precat. nos. 9 and 10; or from Precat. nos. 11 and 12; or Precat. No. 13.

In some embodiments the spray-dried supported catalyst system is selected from SCS nos. 1 to 13; or from any thirteen of SCS nos. 1 to 13; or from SCS nos. 1 to 9, 11, and 12; or from SCS nos. 1 and 2; or from SCS nos. 3 and 4; or from SCS nos. 5 and 6; or from SCS nos. 7 and 8; or from SCS nos. 9 and 10; or from SCS nos. 11 and 12, or SCS no. 13. In some embodiments the supported catalyst system is an undried supported catalyst system selected from SCS nos. 14 to SCS 16; or from any three of SCS nos. 14 to SCS 16; or from SCS nos. 14 and 15; or SCS no. 16.

Embodiments also include a method of making a polyolefin in a gas phase polymerization process, the method comprising contacting one or more olefin monomers with the supported catalyst system, described above, in a gas phase polymerization reactor under gas phase polymerization conditions to make a polyolefin polymer. In some embodiments the one or more olefin monomers comprise ethylene or propylene and optionally a 1-alkene having from 4 to 20 carbon atoms (“(C₄-C₂₀)1-alkene”) and the polyolefin polymer that is made comprises a polyethylene polymer selected from a polyethylene homopolymer or an ethylene/(C₄-C₂₀)1-alkene copolymer or a polypropylene polymer selected from a polypropylene homopolymer or a propylene/(C₄-C₂₀)1-alkene copolymer. In some embodiments the one or more olefin monomers comprises ethylene and 1-butene, 1-hexene, or 1-octene and the polyethylene polymer is an ethylene/1-butene copolymer, an ethylene/1-hexene copolymer, or an ethylene/1-octene copolymer. In some of the foregoing embodiments making the polyethylene polymer, the polyethylene polymer has a weight-average molecular weight greater than 1,000,000 grams per mole and/or a z-average molecular weight greater than 2,000,000 grams per mole.

In some embodiments the method of polymerizing comprises gas phase polymerization and has any one of limitations (i) to (v): (i) wherein the one or more olefin monomers comprises ethylene or a combination of ethylene and propylene or a combination of ethylene and a (C₄-C₂₀)alpha-olefin and wherein the polyolefin polymer is an ethylene homopolymer or an ethylene/propylene copolymer or an ethylene/(C₄-C₂₀)alpha-olefin copolymer; (ii) wherein the one or more olefin monomers comprises ethylene or a combination of ethylene and a (C₄-C₂₀)alpha-olefin and the polyolefin polymer is an ethylene homopolymer or an ethylene/(C₄-C₂₀)alpha-olefin copolymer; wherein the ethylene homopolymer or an ethylene/(C₄-C₂₀)alpha-olefin copolymer has a weight-average molecular weight 1,000,000 grams per mole or greater, or a z-average molecular weight of 2,000,000 grams per mole or greater, or both; or (iii) wherein the one or more olefin monomers comprises a combination of ethylene and a (C₄-C₂₀)alpha-olefin and wherein the polyolefin polymer is an ethylene/(C₄-C₂₀)alpha-olefin copolymer having a polydispersity index (PDI) of a ratio of weight-average molecular weight to number-average molecular weight (Mw/Mn) of greater than or equal to 4.0, or a broad molecular weight distribution of a ratio of z-average molecular weight to weight-average molecular weight (Mz/Mw) greater than or equal to 3.5, or both; (iv) any one of limitations (i) to (iii) wherein the (C₄-C₂₀)alpha-olefin is 1-hexene; (v) a combination of limitations (ii) and (iii) or a combination of limitations (ii), (iii), and (iv).

Embodiments also include a method of making a polyolefin in a slurry phase polymerization process, the method comprising contacting one or more olefin monomers with the supported catalyst system, described above, in a slurry phase polymerization reactor under slurry phase polymerization conditions to make a polyolefin polymer. In some embodiments the method has any one of limitations (i) and (ii): (i) wherein the one or more olefin monomers comprises ethylene or a combination of ethylene propylene or a combination of ethylene and a (C₄-C₂₀)alpha-olefin and the polyolefin polymer comprises an ethylene homopolymer or an ethylene/propylene copolymer of an ethylene/(C₄-C₂₀)alpha-olefin copolymer. In some embodiments the one or more olefin monomers comprises a combination of ethylene and 1-hexene and the polyolefin polymer comprises an ethylene/1-hexene copolymer.

### Synthesis of the Compound of Formula (I) and the Precatalyst of Formula (II)

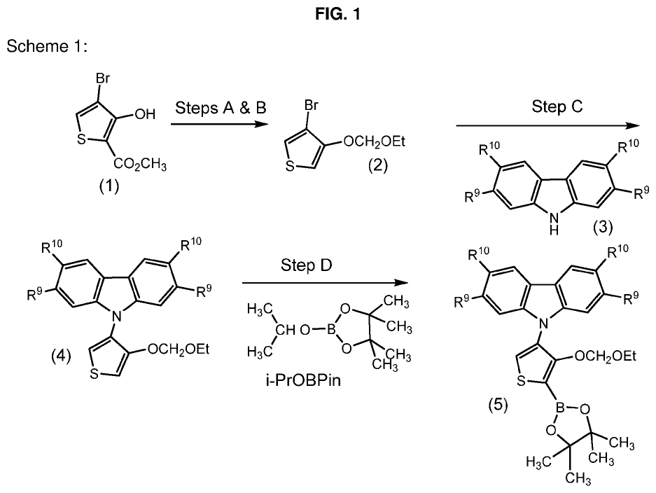

The compound of formula (I) and the precatalyst of formula (II) can be synthesized according to Schemes 1 to 4 shown in FIGS. 1 to 4. Reaction workup procedures are standard and described later in the Examples. Structures are characterized by proton-nuclear magnetic resonance (¹H-NMR) spectroscopy and carbon-13 nuclear magnetic resonance (¹³C-NMR) spectroscopy.

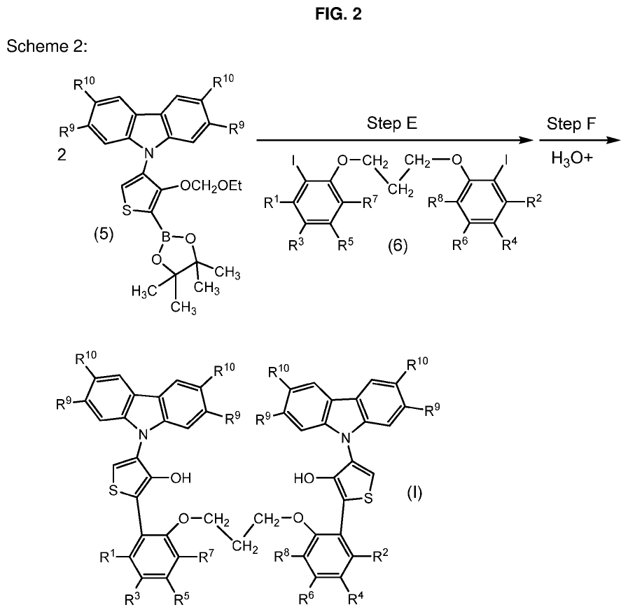

FIG. 1 depicts synthetic Scheme 1 showing the conversion of starting material (1) to intermediate compound (5). In Scheme 1, 3-bromo-2-hydroxy-thiophene-1-carboxylic acid methyl ester (1) was obtained from a commercial supplier. In step A, compound (1) is saponified with sodium hydroxide (NaOH) in aqueous 1,4-dioxane at 80° C. to give 3-bromo-2-hydroxy-thiophene-1-carboxylate sodium salt. The carboxylate was heated with concentrated hydrochloric acid at 60° C. to give 3-bromo-2-hydroxythiophene. In step B the 3-bromo-2-hydroxythiophene was reacted with lithium hydroxide monohydrate (LiOH·H₂O), ethoxychloromethane (ClCH₂OCH₂CH₃) in 1,4-dioxane/tetrahydrofuran (1:4, v/v) at 0° C. to make 3-bromo-2-ethoxymethyloxythiophene (2). In step C, 1.0 mole equivalent of compound (2) was reacted with 2.20 mole equivalents of 3,6-di-t-butylcarbazole (3), 2.00 mole equivalents of cuprous oxide (Cu₂O), 10 mole equivalents of potassium carbonate (K₂CO₃), and 4.0 mole equivalents of N,N′-dimethylethylenediamine (“DMEDA”) in deoxygenated anhydrous xylenes at 140° C. to make the 2-ethoxymethyloxy-3-carbazolyllthiophene (4). In step D, 1 mole equivalent of compound (4) was reacted with 1.25 mole equivalents of n-butyl lithium at −35° C. for 4 hours, and then 2.0 mole equivalents of neat isopropoxyboropinacolate ester (“i-PrOBPin”) were added and the temperature allowed to warm to room temperature to make intermediate compound (5). The synthesis continues in Scheme 2 shown in FIG. 2.

FIG. 2 depicts synthetic Scheme 2 showing the conversion of intermediate compound (5) to the substituted 2-hydroxythiophene compound of formula (I). In Step E, 3 mole equivalents of compound (5) were reacted with 1 mole equivalent of a 1,3-bis(iodophenoxy)propylene (6) wherein R¹, R², R³, R⁴, R⁵, R⁶, R⁷, and R⁸ are as defined for formula (I) in the presence of a catalyst (e.g., bis(di-tert-butyl(4-dimethylaminophenyl)phosphine)dichloropalladium(II)) or “Pd(AmPhos)Cl₂”) and 9 mole equivalents of potassium phosphate tribasic (K₃PO₄) to make a bis(ethoxymethyl)-protected compound. The bis(ethoxymethyl)-protected compound was used in Step F comprising deprotective hydrolysis with concentrated hydrochloric acid in dichloromethane/1,4-dioxane (1:1, v/v) under nitrogen at 23° C. to make the substituted 2-hydroxythiophene compound of formula (I).

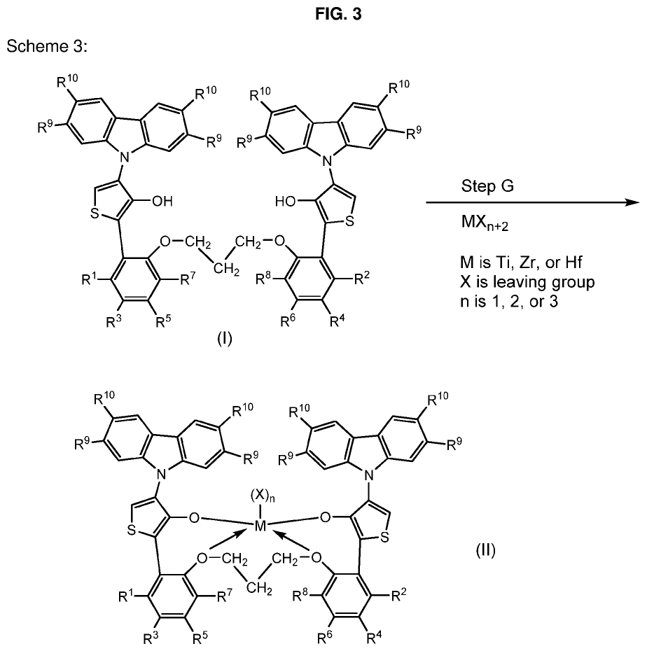

FIG. 3 depicts synthetic Scheme 3 showing the conversion of the substituted 2-hydroxythiophene compound of formula (I) to an embodiment of the precatalyst of formula (11). In Step G, 1.0 mole equivalent of the substituted 2-hydroxythiophene compound of formula (I) is azeotropically dried using toluene.. Then, to a solution of compound (1) in toluene was added 1.15 mole equivalent of a Group 4 metal salt of formula M(X)n+2, wherein M, X, and subscript n are as defined for formula (II), dropwise, and the reaction mixture was stirred at 23° C. for 30 minutes. The reaction mixture was filtered through a 0.45 micrometer (μm) polytetrafluoroethylene filter, and the filtrate was concentrated to give the precatalyst of formula (II). Examples of the Group 4 metal salt of formula M(X)ₙ₊₂ are zirconium tetrachloride (ZrCl₄), zirconium dibenzyl dichloride (ZrBn₂Cl₂), zirconium tetrabenzyl (ZrBn₄), hafnium tetrachloride (HfCl₄), hafnium dibenzyl dichloride (HfBn₂Cl₂), and hafnium tetrabenzyl (HfBn₄). Benzyl, abbreviated “Bn”, is phenylmethyl, which is a monoradical of formula —CH₂C₆H₅. The ZrCl₄, ZrBn₂Cl₂, HfCl₄, or HfBn₂Cl₂ make embodiments of the precatalyst of formula (II) wherein M is Zr or Hf and each X is Cl. These embodiments may be converted to other embodiments of the precatalyst of formula (II). For example, the embodiments of the precatalyst of formula (II) wherein each X is Cl may be reacted with n mole equivalents of an alkylmagnesium halide or of an alkyl lithium to make the precatalyst of formula (II) wherein each X is alkyl. The embodiments of the precatalyst of formula (II) wherein M is Zr or Hf and each X is benzyl (Bn) are made directly from the ZrBn₄ or HfBn₄. Alternatively, the embodiments of the precatalyst of formula (II) wherein M is Zr or Hf and each X is benzyl (Bn) may be made from the embodiments of the precatalyst of formula (II) wherein M is Zr or Hf and each X is Cl by reacting them with n mole equivalents of a benzylmagnesium halide or benzyl lithium.

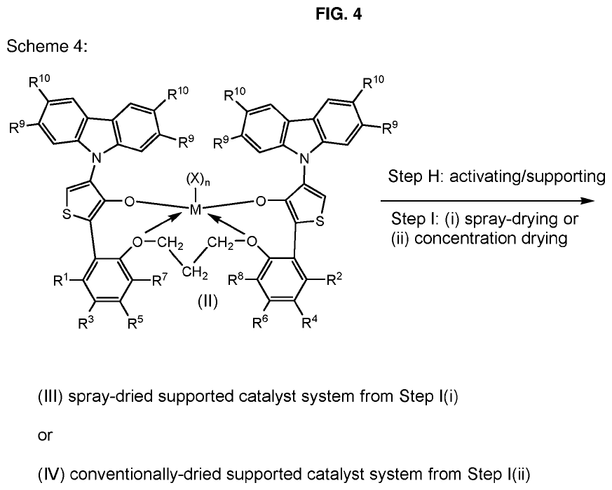

FIG. 4 depicts synthetic Scheme 4. In Step H of Scheme 4, the precatalyst of formula (II) is activated by an activator and supported on a support material in an inert hydrocarbon liquid, such as alkanes or toluene, to make a supported catalyst system suspended in the inert hydrocarbon liquid. In Step I(i), the suspension of the supported catalyst system is spray-dried as described herein to make a spray-dried supported catalyst system (“sd-SCS”) embodiment. Alternatively, in Step I(ii) the suspension of the supported catalyst system is conventionally dried as described herein to make a conventionally-dried supported catalyst system (“cd-SCS”) embodiment. The catalyst activity and catalyst productivity of an sd-SCS embodiment made from a given precatalyst of formula (II) are different than, and typically superior to, the catalyst activity and catalyst productivity of a cd-SDS embodiment made from the same precatalyst of formula (II).

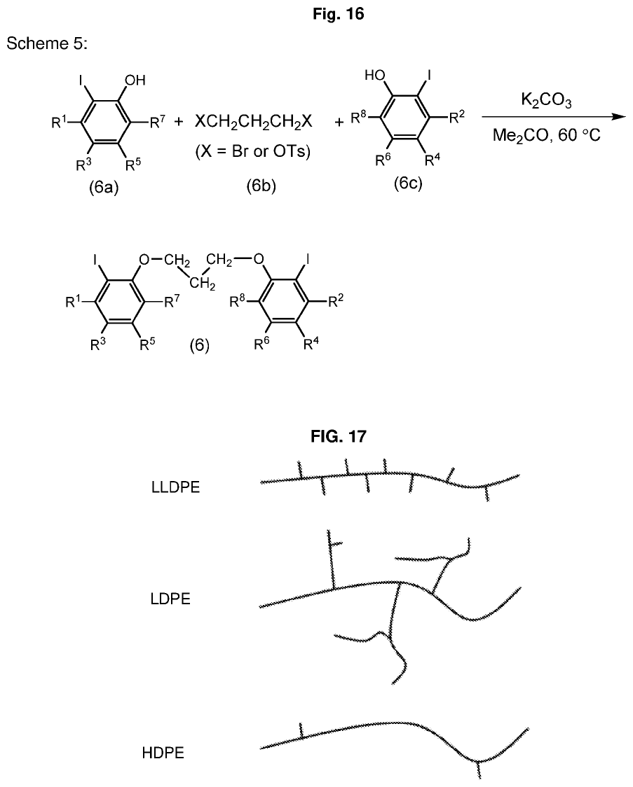

FIG. 16 depicts synthetic Scheme 5. In Scheme 5, the 1,3-bis(iodophenoxy)propylene compound (6) shown in Scheme 2 in FIG. 2 is made from phenols (6a) and (6c) and 1,3-dibromopropane or 1,3-bistosylatepropane (6b) in the presence of potassium carbonate (K₂CO₃) in acetone (Me₂CO) at 60° C.

### Supported Catalyst System

The supported catalyst system is a heterogeneous olefin polymerization catalyst. The heterogenization of the precatalyst of formula (II) with support material and activator to make the supported catalyst system may be carried out according to any one of the heterogenization routes described earlier. The supported catalyst system is formulated for use in gas phase or slurry phase polymerizations of olefin monomers.

The supported catalyst system is made from the precatalyst of formula (II), an activator, and a solid support. The supported catalyst system may comprise additional components such as by-products and side products of the preparation of the supported catalyst system and any unreacted activator that may remain in preparations that use an excess amount of activator relative to the amount of the precatalyst of formula (II).

The catalysts of the supported catalyst system may be unsupported when contacted with an activator, which may be the same or different for the different catalysts. Alternatively, the catalysts may be disposed by spray-drying onto a solid support material prior to being contacted with the activator(s). The solid support material may be uncalcined or calcined prior to being contacted with the catalysts. The solid support material may be a hydrophobic fumed silica (e.g., a fumed silica treated with dimethyldichlorosilane, which is ((CH₃)₂SiCl₂), which is commercially available from Cabot Corporation as Cabosil™ TS-610 fumed silica. The bimodal (unsupported or supported) catalyst system may be in the form of a powdery, free-flowing particulate solid.

### Support Material

The support material used in the supported catalyst system may be an inorganic oxide solid. The terms “support”, “solid support”, “support material”, and “solid support material” mean the same thing as used herein and refer to a porous inorganic substance or organic substance. In some embodiments, the support material may be an inorganic oxide, which includes Group 2, 3, 4, 5, 13 or 14 metal oxides, alternatively Group 13 or 14 metal oxides. Examples of inorganic oxide-type support materials are silica, magnesia, alumina, titania, zirconia, thoria, and mixtures of any two or more of such inorganic oxides. Examples of such mixtures are silica-chromium, silica-alumina, and silica-titania. The support material may be untreated or the support material may be treated with a hydrophobing agent. In some embodiments the support material is a hydrophobic fumed silica.

The inorganic oxide support material is porous and has variable surface area, pore volume, and average particle size. In some embodiments, the surface area is from 50 to 1000 square meter per gram (m²/g) and the average particle size is from 1 to 300 micrometers (μm), alternatively 20 to 300 μm. Alternatively, the pore volume is from 0.5 to 6.0 cubic centimeters per gram (cm³/g) and the surface area is from 200 to 600 m²/g. Alternatively, the pore volume is from 1.1 to 1.8 cm³/g and the surface area is from 245 to 375 m²/g. Alternatively, the pore volume is from 2.4 to 3.7 cm³/g and the surface area is from 410 to 620 m²/g. Alternatively, the pore volume is from 0.9 to 1.4 cm³/g and the surface area is from 390 to 590 m²/g. Each of the above properties are measured using conventional techniques known in the art.

The support material may comprise silica, alternatively amorphous silica (not quartz), alternatively a high surface area amorphous silica (e.g., from 500 to 1000 m²/g). Such silicas are commercially available from several sources including the Davison Chemical Division of W.R. Grace and Company (e.g., Davison 952 and Davison 955 products), and PQ Corporation (e.g., ES70 product). The silica may be in the form of spherical particles, which may be obtained by a spray-drying process. Alternatively, MS3050 product is a silica from PQ Corporation that is not spray-dried. As procured, these silicas are not calcined (i.e., not dehydrated). Silica that is calcined prior to purchase may also be used as the support material.

In some embodiments the solid support is a hydrophobic fumed silica. The hydrophobic fumed silica is made by contacting an untreated fumed silica, having surfaces containing silicon-bonded hydroxyl groups (Si-OH groups), with a hydrophobing agent, described later. In some embodiments the hydrophobing agent is a silicon-based hydrophobing agent, containing on average per molecule one or more functional groups reactive with a Si—OH group, to give the hydrophobic fumed silica. The silicon-based hydrophobing agent may be selected from (CH₃)₂SiCl₂, a polydimethylsiloxane, hexamethyldisilazane (HMDZ), and a (C₁-C₁₀)alkylSi((C₁-C₁₀)alkoxy)₃ (e.g., an octyltrialkoxysilane such as octyltriethoxysilane, i.e., CH₃(CH₂)₇Si(OCH₂CH₃)₃). In some embodiments the silicon-based hydrophobing agent is dimethyldichlorosilane, i.e., (CH₃)₂SiCl₂. In some embodiments the support material is a dimethyldichlorosilane-treated fumed silica, such as that sold as product TS-610 from Cabot Corporation.

The support material may be uncalcined or calcined. The calcined support material is made prior to being contacted with a precatalyst, activator, and/or hydrophobing agent, by heating the support material in air to give a calcined support material. The calcining comprises heating the support material at a peak temperature from 3500 to 850° C., alternatively from 400° to 8000 C., alternatively from 400° to 700° C., alternatively from 500° to 6500 C. and for a time period from 2 to 24 hours, alternatively from 4 to 16 hours, alternatively from 8 to 12 hours, alternatively from 1 to 4 hours, thereby making the calcined support material. If the support material has not been heated in this way it is an uncalcined support material.

### Hydrophobing Agent

The hydrophobing agent is an organic compound or an organosilicon compound that forms a stable reaction product with surface hydroxyl groups of a fumed silica. The organosilicon compound may be a polydiorganosiloxane compound or an organosilicon monomer, which contains silicon bonded leaving groups (e.g., Si-halogen, Si-acetoxy, Si-oximo (Si—ON═C<), Si-alkoxy, or Si-amino groups) that react with surface hydroxyl groups of untreated fumed silica to form Si—O—Si linkages with loss of water molecule as a by-product. The polydiorganosiloxane compound, such as a polydimethylsiloxane, contains backbone Si—O—Si groups wherein the oxygen atom can form a stable hydrogen bond to a surface hydroxyl group of fumed silica. The silicon-based hydrophobing agent may be trimethylsilyl chloride, dimethyldichlorosilane, a polydimethylsiloxane fluid, hexamethyldisilazane, an octyltrialkoxysilane (e.g., octyltrimethoxysilane), and a combination of any two or more thereof.

### Activator

The activator used in the heterogenization method may be any compound capable of reacting with the precatalyst of formula (II) to yield an active olefin polymerization catalyst. The activator may be a Lewis acid, a non-coordinating ionic activator, or an ionizing activator, or a Lewis base.

In some embodiments the activator is an aluminum based activator. The molar ratio of activator's metal (AI) to a particular catalyst compound's metal (Group 4 metal, e.g., Ti, Zr, or Hf) may be 7,000:1 to 0.5:1, alternatively 3,500:1 to 1:1, alternatively 1,000:1 to 0.5:1, alternatively 300:1 to 1:1, alternatively 150:1 to 1:1. Suitable activators are commercially available. In some embodiments the aluminum based activator is an alkylaluminum or an alkylaluminoxane (alkylalumoxane). Any alkyl group may be used. In some embodiments each alkyl of the alkylaluminum or alkylaluminoxane independently may be a (C₁-C₈)alkyl, alternatively a (C₁-C₇)alkyl, alternatively a (C₁-C₆)alkyl, alternatively a (C₁-C₄)alkyl.

The alkylaluminum may be a trialkylaluminum, alkylaluminum halide, or alkylaluminum alkoxide (diethylaluminum ethoxide). The trialkylaluminum may be trimethylaluminum, triethylaluminum (“TEAI”), tripropylaluminum, or tris(2-methylpropyl)aluminum. The alkylaluminum halide may be diethylaluminum chloride. The alkylaluminum alkoxide may be diethylaluminum ethoxide.

The alkylaluminoxane may be a methylaluminoxane (MAO), ethylaluminoxane, 2-methylpropyl-aluminoxane, or a modified methylaluminoxane (MMAO).

In some embodiments the activator is the MAO.

### Supported Catalyst System

Once the precatalyst of formula (II) and the activator are in contact with each other, whether in the presence or absence of the solid support, as the case may be for the first, second or third heterogenization routes described earlier, an active catalyst species and an activator species are made in situ. The active catalyst species comprises a ligand derived from the substituted 2-hydroxythiophene compound of formula (I) and an activator species. The activator species has a different structure or composition than the activator from which it is derived. The activation reaction may also generate one or more by-products. The corresponding activator species may be a derivative of the Lewis acid, non-coordinating ionic activator, ionizing activator, Lewis base, alkylaluminum, or alkylaluminoxane, respectively. An example of the derivative of the by-product is a methylaluminoxane species that is formed by devolatilizing during spray-drying of a supported catalyst system made with methylaluminoxane.

The supported catalyst system may be made by the heterogenization routes described earlier. These routes typically include use of an inert hydrocarbon liquid as solvent or carrier.

In some embodiments the precatalyst and support material are contacted together in the inert hydrocarbon liquid to give a suspension of a supported precatalyst in the inert hydrocarbon liquid, then the suspension is contacted with the activator to give a suspension of the supported catalyst system in the inert hydrocarbon liquid, and then the inert hydrocarbon liquid is removed to give the supported catalyst system.

In other embodiments the precatalyst and activator are contacted together in an inert hydrocarbon liquid to give a solution of a catalyst in the inert hydrocarbon liquid, then the solution is contacted with the support material to give a suspension of the supported catalyst system in the inert hydrocarbon liquid, and then the inert hydrocarbon liquid is removed to give the supported catalyst system.

In other embodiments the activator and the support material are contacted together in an inert hydrocarbon liquid to give a suspension of a supported activator in the inert hydrocarbon liquid, then the suspension is contacted with the precatalyst to give a suspension of the supported catalyst system in the inert hydrocarbon liquid, and then the inert hydrocarbon liquid is removed to give the supported catalyst system.

In other embodiments the precatalyst, activator, and support material are contacted together simultaneously in an inert hydrocarbon liquid to give a suspension of the supported catalyst, and then the inert hydrocarbon liquid is removed to give the supported catalyst system.

The removing of the inert hydrocarbon liquid from the suspension of the supported catalyst system may include a step of decanting some of the inert hydrocarbon liquid from the suspension. In some embodiments the decanting method comprises pouring off excess inert hydrocarbon liquid from the suspension to give a concentrated suspension of the supported catalyst system.

The removing of the inert hydrocarbon liquid from the suspension of the supported catalyst system may comprise a step of drying the supported catalyst system. The drying step may comprise a conventional drying method or a spray-drying method.

The conventional drying method comprises a method of slowly increasing the mass or molar amount of less volatile chemical constituent(s) per unit volume of a continuous mixture comprising more volatile and less volatile chemical constituent(s) by gradually removing the more volatile chemical constituent(s) from the less volatile constituent(s) of the continuous mixture to give a concentrate having a higher mass or molar amount of the less volatile chemical constituent(s) per unit volume than did the continuous mixture, wherein the rate of gradual removing is limited by a relatively small evaporative surface area to mass ratio (compared to spray-drying). The concentrate may be a precipitated solid.

The spray-drying method comprises rapidly forming a particulate solid comprising less volatile chemical constituents via aspiration of a bulk mixture of the less volatile chemical constituents and more volatile chemical constituents through a nebulizer using a hot gas, wherein the aspiration forms particulates collectively having a large evaporative surface area to mass ratio compared to that of concentrating. The particle size and shape of the particulate solid formed by spray-drying may be different than those of a precipitated solid.

In some embodiments the spray-dried supported catalyst system may be made at laboratory scale according to the following spray-drying procedure in a nitrogen-purged glove box: charge an oven-dried glass jar with anhydrous deoxygenated toluene and a solid support material. The contents are stirred at room temperature until well dispersed as a slurry. To the slurry is added a 10% solution by weight of methylaluminoxane (MAO) in toluene. The resulting mixture is stirred for 15 minutes, then a quantity of the precatalyst of formula (II) is added. The resulting reaction mixture is stirred at room temperature for an additional 30 to 60 minutes to activate the precatalyst, yielding the supported catalyst system suspended in toluene. This suspension is spray-dried using a spray drier apparatus (e.g., a BOchi Mini Spray Dryer model B-290 from BUCHI Corporation, New Castle, Delaware, USA) with the following parameters: Set Temperature 1400 C., Outlet Temperature 750 C. (minimum), aspirator setting 95 rotations per minute (rpm), and pump speed 150 rpm. The spray-drying process yields the spray-dried supported catalyst system as an anhydrous solid powder. In some embodiments the solid support material that has been treated with a hydrophobing agent, such as a hydrophobic fumed silica that has been treated with dimethyldichlorosilane. The foregoing procedure may be scaled up to manufacturing size quantities using generally known methods. Comparing Advantages of Undried, Conventionally-Dried, and Spray-Dried Embodiments of the Supported Catalyst System

The present invention contemplates both the conventionally dried supported catalyst system embodiments, the spray-dried supported catalyst system embodiments, and the decanted but undried supported catalyst system embodiments. The decanted but undried supported catalyst system embodiments are useful in catalyzing slurry phase polymerizations and are convenient form for adding the supported catalyst system to a slurry phase reactor.

The conventionally dried supported catalyst system embodiments may have higher catalyst efficiencies, and thus greater polyolefin productivities, than do comparative unsupported catalysts made from the same precatalyst and activator in the absence of the support material.

The spray-dried supported catalyst system embodiments may have higher catalyst efficiencies, and thus greater polyolefin productivities, than do the conventionally dried supported catalyst system embodiments. Therefore, the spray-dried embodiments of the supported catalyst system may have still higher catalyst efficiencies, and thus still greater polyolefin productivities, than do comparative unsupported catalysts made from the same precatalyst and activator in the absence of the support material.

Many spray-dried supported catalyst system embodiments also make polyolefins having improved resin properties. For example, some spray-dried supported catalyst system embodiments make polyolefins having increased content of long chain branching (LCB), whereas other spray-dried supported catalyst system embodiments and the conventionally dried catalyst system embodiments do not. In another example, some spray-dried supported catalyst system embodiments make polyolefins having ultrahigh molecular weight contents, whereas other spray-dried supported catalyst system embodiments and the conventionally dried catalyst system embodiments do not.

The supported catalyst system may be used in slurry phase or gas phase olefin polymerization reactions to enhance the rate of polymerization of monomer and/or comonomer(s). In some aspects the olefin polymerization reaction is conducted in a gas phase reactor in the gas phase, or in a slurry phase reactor in the slurry phase.

### Method of Polymerizing One or More Olefin Monomers

In embodiments of the method of polymerizing an olefin monomer, the method comprising contacting the olefin monomer with the supported catalyst system, thereby making a polyolefin, wherein the olefin polymerization is conducted in a gas phase reactor under gas phase process conditions or the olefin polymerization is conducted in a slurry phase reactor under slurry phase conditions. In some embodiments the method comprises polymerizing ethylene only and makes a polyethylene homopolymer. In other embodiments the method comprises polymerizing ethylene and propylene and makes an ethylene/propylene copolymer, or polymerizing ethylene and a (C₄-C₈)alpha-olefin and makes an ethylene/(C₄-C₈)alpha-olefin copolymer. In some embodiments the (C₄-C₈)alpha-olefin is 1-butene, 1-hexene, or 1-octene; or 1-butene or 1-hexene; or 1-butene; or 1-hexene; or 1-octene; and the ethylene/(C₄-C₈)alpha-olefin copolymer is ethylene/1-butene copolymer, ethylene/1-hexene copolymer, or ethylene/1-octene copolymer; or ethylene/1-butene copolymer or ethylene/1-hexene copolymer; or ethylene/1-butene copolymer; or ethylene/1-hexene copolymer; or ethylene/1-octene copolymer.

### Polymerization Reactors and Process Conditions

The method of polymerizing an olefin monomer may be carried out in any gas phase olefin polymerization reactor or slurry phase olefin polymerization reactor and under any gas phase polymerization process conditions or slurry phase polymerization conditions.

Reactors and process conditions for gas phase and slurry phase olefin polymerization reactions are well-known. For example slurry phase reactors and process conditions include those described in U.S. Pat. No. 3,324,095. The gas phase polymerization reactor and process conditions may employ stirred-bed gas-phase polymerization reactor (SB-GPP reactor) or a fluidized-bed gas-phase polymerization reactor (FB-GPP reactor). The gas phase reactor and process conditions may include an induced condensing agent and be conducted in condensing mode polymerization such as described in U.S. Pat. Nos. 4,453,399; 4,588,790; 4,994,534; 5,352,749; 5,462,999; and 6,489,408. The gas phase reactor and process conditions may be a fluidized bed reactor/method as described in U.S. Pat. Nos. 3,709,853; 4,003,712; 4,011,382; 4,302,566; 4,543,399; 4,882,400; 5,352,749; 5,541,270; EP-A-0 802 202; and Belgian Patent No. 839,380. These patents disclose gas phase polymerization processes wherein the polymerization medium is either mechanically agitated or fluidized by the continuous flow of the gaseous monomer and diluent. Other useful gas phase processes include series or multistage polymerization processes such as described in U.S. Pat. Nos. 5,627,242; 5,665,818; 5,677,375; EP-A-0 794 200; EP-B1-0 649 992; EP-A-0 802 202; and EP-B-634421.

In some embodiments the gas phase reactor and process conditions comprise a single gas phase reactor and single set of process conditions.

In other embodiments the gas phase reactor and process conditions comprise two gas phase reactors in series and two sets of process conditions. In such embodiments a first olefin polymerization is conducted in a first gas phase reactor under a first gas phase process conditions, then the resulting polyolefin is transferred into a second gas phase reactor, wherein a second olefin polymerization reaction is conducted under a second set of process conditions. The supported catalyst system may be used in the first olefin polymerization and not the second olefin polymerization, or in the second olefin polymerization and not the first olefin polymerization, or in both the first and second olefin polymerizations. The supported catalyst system used in both the first and second olefin polymerizations may be the same embodiment or different embodiments.

In some embodiments the olefin polymerization comprises a slurry phase reactor and process conditions and a gas phase reactor and process conditions in series, or vice versa. In some embodiments a first olefin polymerization is conducted in the slurry phase reactor under the slurry phase process conditions, then the slurry phase polyolefin is transferred into the gas phase reactor and a second olefin polymerization is conducted under gas phase conditions. The supported catalyst system may be used in the first olefin polymerization and not the second olefin polymerization, or in the second olefin polymerization and not the first olefin polymerization, or in both the first and second olefin polymerizations. The supported catalyst system used in both the first and second olefin polymerizations may be the same embodiment or different embodiments.

### Polyolefin

The product of the olefin polymerization method is a polyolefin.

In some embodiments the polyolefin is a low-density polyethylene (LDPE), linear low-density polyethylene (LLDPE), a medium-density polyethylene (MDPE), or a high-density polyethylene (HDPE). These polyethylenes have different polymer chain structures, which are pictorially illustrated in FIG. 17, and are a result of the different polymerization process conditions and initiators or catalysts used to make them. In general an LLDPE is distinguished from LDPE by the initiator or catalyst and the polymerization process conditions used to make them, which leads to differences in their amounts of long chain branching. LDPE is made by a free radical polymerization process (e.g., initiated by small amounts of organic peroxide) at high pressure and as such LDPE inherently has a significant amount of long chain branching as shown in FIG. 17. LLDPEs that are made using traditional Ziegler-Natta catalysts, which do not generate long chain branching, are linear and free of long chain branching as illustrated in FIG. 17. In general an LLDPE is distinguished from HDPE by density and by the amount of short chain branching (SCB). LLDPEs have densities less than 0.940 g/cm³, whereas HDPE has densities greater than or equal to 0.940 g/cm³. Also, LLDPEs have a significant amount of short chain branching, whereas HDPEs have far lesser amounts of short chain branching; see FIG. 17.

In some embodiments the polyethylene may have no detectable long-chain branching content, i.e., 0 long-chain branches (“LCB”) per 1000 carbon atoms. In other embodiments the polyethylene may have a long-chain branching content from 0.01 to 2 long-chain branches (“LCB”) per 1000 carbon atoms (LCB/1000C), alternatively from 0.01 LCB/1000C to 1.0 LCB/1000C, alternatively from 0.1 LCB/1000C to 1.0 LCB/1000C. As used herein having a LCB content means having an amount of long chain branching that is detectable by the 13C-NMR spectroscopy, which currently has a lower detection limit of 0.004 LCB/1000C. LCB content from greater than 0.000 LCB/1000C to less than 0.010 LCB/1000C are excluded herein.

The long chain branching content of the inventive polyolefin may be directly or indirectly characterized by any one of the following measurements (i) to (iv): (i) directly by carbon-13 nuclear magnetic resonance (NMR) spectroscopy; (ii) indirectly by a melt flow ratio (121/12) equation described below; (iii) indirectly by a melt flow ratio (121/12) range; or (iv) Mark-Houwink analysis using a triple detector gel permeation chromatography (triple detector GPC). In some embodiments the characterization may comprise a combination of measurements (i) and (ii), a combination of measurements (i) and (iv), a combination of measurements (i) and (iii), a combination of measurements (ii) and (iii), a combination of measurements (ii) and (iv), a combination of measurements (iii) and (iv), or a combination of measurements (i), (ii), (iii), and (iv).

In some embodiments the polyethylene may have ultrahigh molecular weight (“UHMW”) content. In some embodiments the polyethylene may have UHMW tail in a GPC plot. The UHMW content of these polyethylene embodiments may be measured by GPC, and is a polymer weight average molecular weight of 1,000,000 g/mol or greater. The UHMW tail is any one of limitations (i) to (iii): (i) a z-average molecular weight of 1,000,000 g/mol or greater, (ii) a ratio of z-average molecular weight to weight-average molecular weight (Mz/Mw) of 3.5 or greater, or (iii) both limitations (i) and (ii).

The polyolefin may be formulated with one or more additives useful in polyethylene articles, such as but not limited to, additives useful in polyethylene films, additives useful in polyethylene pipes, or additives useful in blow molded polyethylene articles. In some embodiments the one or more additives comprise additives useful for films such as one or more antioxidants, one or more ultraviolet (UV) light stabilizers, one or more colorants, and/or one or more anti-microbial agents.

### Definitions

Activator a compound for converting a precatalyst having no or negligible catalytic activity into a catalyst having orders of magnitude higher catalytic activity.

Alpha-olefin: is a terminal monoalkene of formula H₂C═CH(CH₂)ₖCH₃ wherein subscript k is an integer of 0 or higher, or 1 or higher; abbreviated “α-olefin”.

Biphenyl: a compound of this structure and position numbering:

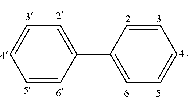 [SMILES: c1ccc(-c2ccccc2)cc1]

Carbazole: is a compound of structure and position numbering:

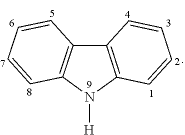 [SMILES: c1ccc2c(c1)[nH]c1ccccc12]

Dry: precatalysts, activators, catalysts and calcined support materials may have a moisture content from 0 to less than 5 parts per million based on total parts by weight. Dry may also refer to being free of an organic solvent such as toluene or hexanes when used to describe an embodiment of a supported catalyst system as a dry powder.

Fumed silica: a pyrogenic silica produced in a flame. An amorphous silica powder made by fusing microscopic droplets into branched, chainlike, three-dimensional secondary particles, which agglomerate into tertiary particles. Not quartz.

Heteroatoms: as used herein, generic heteroatom-containing organic groups wherein the specific heteroatom or heteroatoms is not or are not explicitly or implicitly indicated, such as is the case for “heterohydrocarbyl” groups and “organoheteryl” groups, inherently contain one or more heteroatoms selected from the group consisting of O, S, N, P, and Si; or O, S, N, and Si; or O, N, and Si; or O and N; or O; or N; or Si: or S; or P. In contrast, examples of heteroatom-containing organic groups wherein the heteroatom is explicitly or implicitly indicated are: alkoxy groups wherein the heteroatom implicitly is O′ and amino groups wherein the heteroatom implicitly is N; alkylO-groups wherein the heteroatom explicitly is O; and —CH₂Si(alkyl)₃ groups wherein the heteroatom explicitly is Si.

Hydrocarbyl, heterohydrocarbyl, and organoheteryl have their IUPAC Gold Book meanings. The hydrocarbyl is a monovalent radical that in unsubstituted embodiments consists of one or more carbon atoms and hydrogen atoms, wherein the monovalent radical is on a carbon atom. Examples are alkyl and aryl. The heterohydrocarbyl group is a monovalent radical that in unsubstituted embodiments consists of one or more carbon atoms and at least one heteroatom, wherein the monovalent radical is a carbon atom. Examples are ethoxymethyl and —CH₂Si(alkyl)₃. The organoheteryl group is a monovalent radical that in unsubstituted embodiments consists of one or more carbon atoms and at least one heteroatom, wherein the monovalent radical is a heteroatom. Examples alkoxy and —Si(alkyl)₃.

Inert: not (appreciably) reactive. The term “inert” as applied to the purge gas or olefin monomer feed means a molecular oxygen (O₂) content from 0 to less than 5 parts per million based on total parts by weight of the purge gas or olefin monomer feed. As applied to a hydrocarbon (unsubstituted) solvent means free of carbon-carbon double and triple bonds, free of molecular oxygen (O to less than 5 ppm O₂), and free of moisture (“dry”, 0 to less than 5 ppm H₂O). Examples are hydrocarbon solvents that may be inerted (dried and purged of O₂) are unsubstituted alkanes (e.g., hexanes and heptane), unsubstituted arenes (e.g., benzene and naphthalene), and unsubstituted alkylarenes (e.g., toluene, xylenes, and fluorene).

Metallocene catalyst. Homogeneous or heterogeneous molecule that contains an unsubstituted- or substituted-cyclopentadienyl ligand-metal complex and enhances olefin polymerization reaction rates. With respect to number of catalytic sites, typically unsupported metallocene catalyst molecules are substantially single site or dual site and supported metallocene catalysts are multi-sited, meaning two or more sites or speciations. The unsubstituted cyclopentadienyl is a monoanion of formula [C₅H₅]⁻. As used herein “substituted cyclopentadienyl” includes monocyclic derivatives of cyclopentadienyl, such as propylcyclopentadienyl and pentamethylcyclopentadienyl, and multicyclic derivatives of cyclopentadienyl, such as bicyclic derivatives indenyl and tetrahydroindenyl and tricyclic derivatives fluorenyl, tetrahydrofluorenyl, and octahydrfluorenyl, and substituted derivatives thereof. Examples of substituted-cyclopentadienyl ligands are unsubstituted indenyl, alkyl-substituted indenyl, unsubstituted 4,5,6,7-tetrahydroindenyl, alkyl-substituted 4,5,6,7-tetrahydroindenyl, unsubstituted fluorenyl, and alkyl-substituted fluorenyl, unsubstituted 1,2,3,4-tetrahydrofluorenyl, alkyl-substituted 1,2,3,4-tetrahydrofluorenyl, unsubstituted 1,2,3,4,5,6,7,8-octahydrofluorenyl, and alkyl-substituted 1,2,3,4,5,6,7,8-octahydrofluorenyl.

Meta-terphenyl: also named 3-phenyl-1,1′-biphenyl, is a compound of this structure and position numbering:

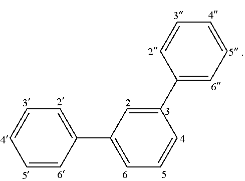 [SMILES: c1ccc(-c2cccc(-c3ccccc3)c2)cc1]

Modality of Molecular Weight Distribution in reference to a polyolefin indicates the nature of the polyolefin's molecular weight distribution greater than a molecular weight of 1,000 grams/mole (Log(MW) >3.0) and less than a molecular weight of 10,000,000 grams/mole (Log(MW)<7.0) in a plot of dW/d Log(MW) on the y-axis versus Log(MW) on the x-axis to give a Gel Permeation Chromatograph (GPC) chromatogram, wherein Log(MW) and dW/d Log(MW) are as defined herein and are measured by the High Temperature Gel Permeation Chromatography (GPC) Test Method described later. Only peaks between log(MW) 3.0 and log(MW) 7.0 count for modality. The modality of the polyolefin may be unimodal (only 1 peak between log(MW) 3.0 and log(MW) 7.0) or multimodal (2 or more peaks between log(MW) 3.0 and log(MW) 7.0). The modality of the multimodal polyolefin may be bimodal (only 2 peaks between log(MW) 3.0 and log(MW) 7.0), trimodal (only 3 peaks between log(MW) 3.0 and log(MW) 7.0), or higher modal (4 or more peaks between log(MW) 3.0 and log(MW) 7.0). Any two peaks between log(MW) 3.0 and log(MW) 7.0 may be separated by a distinguishable local minimum therebetween or one peak may merely be a shoulder on the other. Deconvolution of a GPC plot between log(MW) 3.0 and log(MW) 7.0 may be used to determine if there are any hidden peaks, which would then be counted for modality.

Multi-site catalyst: any catalyst that makes a polyethylene having a polydispersity index (PDI, M_{w}/Mₙ) greater than 2.0.

Olefin monomer: unsubstituted hydrocarbon containing a carbon-carbon double bond.

Polyolefin: a straight chain or branched chain macromolecule consisting of carbon and hydrogen, or plurality of macromolecules, and having six or more constituent units derived by polymerizing an olefin monomer or two or more olefin comonomers.

Precatalyst: a catalyst precursor compound, also called a “precatalyst”. A precatalyst has none or very little catalytic activity itself, but upon being contacted with an activator the precatalyst is converted into a catalyst compound. The precatalyst may be a ligand-metal complex such as the precatalysts described herein.

Prophetic example. An embodiment that has not been actually made, but which can be readily made from the teachings provided herein and which the inventor(s) expect will have the described inventive features and advantages, and is included to support the claimed scope.

Single-site catalyst. An organic ligand-metal complex useful for enhancing rates of polymerization of olefin monomers and having at most two discreet binding sites at the metal available for coordination to an olefin monomer molecule prior to insertion on a propagating polymer chain.

Single-site non-metallocene catalyst. A single-site catalyst that is free of an unsubstituted or substituted cyclopentadienyl ligand.

System (chemical): an interrelated arrangement of different chemical constituents so as to form a functioning whole.

Ziegler-Natta catalyst: a titanium catalyst supported on magnesium dichloride solids, and, optionally, a silica.

In the event of a disagreement between a chemical name and its structure, the structure controls and determines the identity of the compound in question. In the event of a disagreement between a chemical name and its description by reference to formula (I) or (II), and substituent groups R^{#} (e.g., R¹, R², etc.) and X, the description referring to the formula (I) or (II), and substituent R^{#} and X groups controls and determines the identity of the compound in question.

### Experimental Methods

Preparing Test Plaques, Sheets, or Specimens: see ASTM D4703-10, Standard Practice for Compression Molding Thermoplastic Materials into Test Specimens, Plaques, or Sheets.

Density Test Method: measured according to ASTM D792-13, Standard Test Methods for Density and Specific Gravity (Relative Density) of Plastics by Displacement, Method B (for testing solid plastics in liquids other than water, e.g., in liquid 2-propanol). Units are grams per cubic centimeter (g/cm³).

Long Chain Branching (LCB) Value Test Method: the amount of the LCB occurring in the EB LLDPE resins can be measured using a combination of nuclear magnetic resonance (NMR) techniques described in Z. Zhou, S. Pesek, J. Klosin, M. Rosen, S. Mukhopadhyay, R. Cong, D. Baugh, B. Winniford, H. Brown, K. Xu, “Long chain branching detection and quantification in LDPE with special solvents, polarization transfer techniques, and inverse gated ¹³C NMR spectroscopy”, Macromolecules, 2018, 51, 8443; Z. Zhou, C. Anklin, R. Cong, X. Qiu, R. Kuemmerle, “Long-chain branch detection and quantification in ethylene-hexene LLDPE with ¹³C NMR”, Macromolecule, 2021, 54, 757; and Z. Zhou, C. Anklin, R. Kuemmerle, R. Cong, X. Qiu, J. DeCesare, M. Kapur, R. Patel, “Very sensitive ¹³C NMR method for the detection and quantification of long-chain branches in ethylene-hexene LLDPE”, Macromolecule, 2021, 54, 5985. The chemical shift range for LCB calculation is between 38.12 ppm to 38.22 ppm.

Melt Flow Test Methods. Melt flow index values of polyethylenes were measured via the rate of extrusion of molten polymers through a die of specified length and diameter, under prescribed conditions of temperature, load, piston position in the barrel and duration, employing a melt indexer and test methods according to ASTM D1238-13 at 190° C. The load is 2.16 kg (“I₂”), 5.0 kg (“I₅”), or 21.6 kg (“I₂₁”).

Differential Scanning Calorimetry Test Method. Melt temperature was determined via Differential Scanning Calorimetry according to ASTM D 3418-08. In general, a scan rate of 10° C./min on a sample of 10 milligrams (mg) was used, and the second heating cycle was used to determine Tₘ.

### Gel-Permeation Chromatography (GPC) Test Method:

Weight-average molecular weight (M_{w}), number-average molecular weight (Mₙ), and z-average molecular weight (M_{z}) were measured using a High Temperature Gel Permeation Chromatography (Polymer Laboratories), equipped with a differential refractive index detector (DRI). The M_{w} and Mₙ values obtained were used to calculate polydispersity index (PDI), wherein PDI=M_{w}/Mₙ. Three Polymer Laboratories PLgel 10 μm Mixed-B columns were used. The nominal flow rate was 1.0 mL/min, and the nominal injection volume was 300 μL. The various transfer lines, columns, and differential refractometer (the DRI detector) were contained in an oven maintained at 160° C. The solvent for the experiment was prepared by dissolving 6 grams of butylated hydroxytoluene as an antioxidant in 4 liters of Aldrich reagent grade 1, 2, 4 trichlorobenzene (TCB). The TCB mixture was then filtered through a 0.1 μm Teflon filter. The TCB was then degassed with an online degasser before entering the GPC instrument. The polymer solutions were prepared by placing dry polymer in glass vials, adding the desired amount of TCB, then heating the mixture at 160° C. with continuous shaking for about 2 hours. All quantities were measured gravimetrically. The injection concentration was from 0.5 to 2.0 mg/ml, with lower concentrations being used for higher molecular weight samples. Prior to running each sample the DRI detector was purged. The flow rate in the apparatus was then increased to 1.0 ml/minute, and the DRI was allowed to stabilize for 8 hours before injecting the first sample. The molecular weight was determined by combining universal calibration relationship with the column calibration which is performed with a series of monodispersed polystyrene (PS) standards. The MW was calculated at each elution volume with following equation:

$$log {M}_{X} = \frac{log ( {K}_{X} / {K}_{P S} )}{{a}_{X} + 1} + \frac{{a}_{P S} + 1}{{a}_{X} + 1} log {M}_{P S}$$

where the variables with subscript “X” stand for the test sample while those with subscript “PS” stand for PS. In this method, a_{PS}=0.67 and K_{PS}=000017, while aₓ and Kₓ were obtained from published literature. Specifically, a/K=0.695/0.000579 for PE and 0.705/0.0002288 for PP.

The concentration, c, at each point in the chromatogram was calculated from the baseline-subtracted DRI signal, IDRI, using the following equation: c=KDRIIDRI/(dn/dc), where KDRI is a constant determined by calibrating the DRI, and (dn/dc) is the refractive index increment for the system. Specifically, dn/dc=0.109 for polyethylene. The mass recovery was calculated from the ratio of the integrated area of the concentration chromatography over elution volume and the injection mass which is equal to the pre-determined concentration multiplied by injection loop volume. All molecular weights are reported in g/mol unless otherwise noted. In event of conflict between the GPC-DRI procedure and the “Rapid GPC,” the GPC-DRI procedure immediately above shall be used. The comonomer content (i.e., 1-hexene) incorporated in the polymers (weight %)) was determined by rapid FT-IR spectroscopy on the dissolved polymer in a GPC measurement. Comonomer content can be determined with respect to polymer molecular weight by use of an infrared detector such as an IR5 detector in a gel permeation chromatography measurement, as described in Analytical Chemistry 2014, 86(17), 8649-8656. “Toward Absolute Chemical Composition Distribution Measurement of Polyolefins by High-Temperature Liquid Chromatography Hyphenated with Infrared Absorbance and Light Scattering Detectors” by Dean Lee, Colin Li Pi Shan, David M. Meunier, John W. Lyons, Rongjuan Cong, and A. Willem deGroot. Analytical Chemistry 2014 86 (17), 8649-8656.

For some of the polymer samples, weight-average molecular weight (M_{w}), number-average molecular weight (Mₙ), and z-average molecular weight (M_{z}) were measured using a chromatographic system consisting of a PolymerChar GPC-IR (Valencia, Spain) high temperature GPC chromatograph equipped with an internal IR5 infra-red detector (IR5) and 4-capillary viscometer (DV) coupled to a Precision Detectors (Now Agilent Technologies) 2-angle laser light scattering (LS) detector Model 2040. For all absolute Light scattering measurements, the 15 degree angle is used for measurement. The autosampler oven compartment was set at 165° Celsius and the column compartment and detectors were set at 155° Celsius. The columns used were 4 TOSOH TSKgel GMHHR-H (30) HT 30-micron particle size, mixed pore size columns. The chromatographic solvent used was 1,2,4 trichlorobenzene and contained 200 ppm of butylated hydroxytoluene (BHT). The solvent source was nitrogen sparged. The injection volume used was 200 microliters and the flow rate was 1.0 milliliters/minute.

Calibration of the GPC column set was performed with 21 narrow molecular weight distribution polystyrene standards with molecular weights ranging from 580 to 8,400,000 and were arranged in 6 “cocktail” mixtures with at least a decade of separation between individual molecular weights. The standards were purchased from Agilent Technologies. The polystyrene standards were prepared at 0.025 grams in 50 milliliters of solvent for molecular weights equal to or greater than 1,000,000, and 0.05 grams in 50 milliliters of solvent for molecular weights less than 1,000,000. Individually prepared polystyrene standards of 10,000,000 and 15,000,000 g/mol, both from Agilent Technologies, were also prepared, at 0.5 and 0.3 mg/mL respectively. The polystyrene standards were pre-dissolved at 80° C. with gentle agitation for 30 minutes then cooled and the room temperature solution is transferred cooled into the autosampler dissolution oven at 160° C. for 30 minutes. The polystyrene standard peak molecular weights were converted to polyethylene molecular weights using Equation 1 (as described in Williams and Ward, J. Polym. Sci., Polym. Let., 6, 621 (1968)).

M_{polyethylene}=A×(M_{polystyrene})^{B} (EQ1),

wherein M is the molecular weight, A has a value of 0.3992, and B equals 1.0.

A third order polynomial was used to fit the respective polyethylene-equivalent calibration points.

The total plate count of the GPC column set was performed with decane which was introduced into blank sample via a micropump controlled with the PolymerChar GPC-IR system. The plate count for the chromatographic system should be greater than 12,000 for the 4 TOSOH TSKgel GMHHR-H (30) HT 30-micron particle size, mixed pore size columns.

Samples were prepared in a semi-automatic manner with the PolymerChar “Instrument Control” Software, wherein the samples were weight-targeted at 1 mg/ml, and the solvent (contained 200 ppm BHT) was added to a pre nitrogen-sparged septa-capped vial, via the PolymerChar high temperature autosampler. The samples were dissolved for 3 hours at 165° Celsius under “low speed” shaking.

The calculations of Mn_{(GPC)}, Mw_{(GPC)}, and Mz_{(GPC)} were based on GPC results using the internal IR5 detector (measurement channel) of the PolymerChar GPC-IR chromatograph according to Equations 2 to 4, using PolymerChar GPCOne™ software, the baseline-subtracted IR chromatogram at each equally-spaced data collection point (i), and the polyethylene equivalent molecular weight obtained from the narrow standard calibration curve for the point (i) from Equation 1.

$${Mn}_{( GPC )} = \frac{{\sum }^{i} {IR}_{i}}{{\sum }^{i} ( \frac{{IR}_{i}}{{M}_{{polyethylene}_{i}}} )} . \qquad ( EQ2 )$$

$${Mw}_{( GPC )} = \frac{{\sum }^{i} ( {IR}_{i} * {M}_{{polyethylene}_{i}} )}{{\sum }^{i} {IR}_{i}} . \qquad ( EQ3 )$$

$${Mz}_{( GPC )} = \frac{{\sum }^{i} ( {IR}_{i} * {M}_{{polyethylene}_{i}}^{2} )}{{\sum }^{i} ( {IR}_{i} * {M}_{{polyethylene}_{i}} )} . \qquad ( EQ4 )$$

In order to monitor the deviations over time, a flowrate marker (decane) was introduced into each sample via a micropump controlled with the PolymerChar GPC-IR system. This flowrate marker (FM) was used to linearly correct the pump flowrate (Flowrate(nominal)) for each sample by RV alignment of the respective decane peak within the sample (RV(FM Sample)) to that of the decane peak within the narrow standards calibration (RV(FM Calibrated)). Any changes in the time of the decane marker peak are then assumed to be related to a linear-shift in flowrate (Flowrate(effective)) for the entire run. After calibrating the system based on a flow marker peak, the effective flowrate (with respect to the narrow standards calibration) is calculated as Equation 5. Processing of the flow marker peak was done via the PolymerChar GPCOne™ Software. Acceptable flowrate correction is such that the effective flowrate should be within +/−0.5% of the nominal flowrate.

$$Flowrate ( effective ) = Flowrate ( nominal ) * ( RV ( FM Calibrated ) / RV ( FM S ample ) ) . \qquad ( EQ 5 )$$

For polymer samples prepared via slurry polymerization in a parallel pressure reactor (PPR), high temperature GPC analysis was performed using a Dow Robot Assisted Delivery (RAD) system equipped with a PolymerChar infrared detector (IR5) and Agilent PLgel Mixed A columns. Decane (10 μL) was added to each sample for use as an internal flow marker. Samples were first diluted in 1,2,4-trichlorobenzene (TCB) stabilized with 300 ppm butylated hydroxyl toluene (BHT) at a concentration of 10 mg/mL and dissolved by stirring at 160° C. for 120 minutes. Prior to injection samples were further diluted with TCB stabilized with BHT to a concentration of 2 mg/mL. Samples (250 μL) were eluted through one PL-gel 20 μm (50×7.5 mm) guard column followed by two PL-gel 20 μm (300×7.5 mm) Mixed-A columns maintained at 160° C. with TCB stabilized with BHT at a flowrate of 1.0 mL/min. The total run time was 24 minutes. To calibrate for molecular weight (MW) Agilent EasiCal polystyrene standards (PS-1 and PS-2) were analyzed to create a 3^{rd} order MW calibration curve. Molecular weight units were converted from polystyrene (PS) to polyethylene (PE) using a daily Q-factor calculated around 0.4 using the average of 5 Dow 38-4 reference samples of known MW. Hexene incorporation was determined by use of a linear calibration developed by analyzing copolymer samples with known compositions.

Liquid chromatography-mass spectrometry (LC-MS) Measurements were performed using a Waters e2695 Separations Module coupled with a Waters 2424 ELS detector, a Waters 2998 PDA detector, and a Waters 3100 ESI mass detector. LC-MS separations were performed on an XBridge C18 3.5 μm 2.1×50 mm column using a 5:95 to 100:0 acetonitrile to water gradient with 0.1% formic acid as the ionizing agent. HRMS analyses were performed using an Agilent 1290 Infinity LC with a Zorbax Eclipse Plus C18 1.8 μm 2.1×50 mm column coupled with an Agilent 6230 TOF Mass Spectrometer with electrospray ionization.

Nuclear magnetic resonance (NMR) spectra were recorded on Bruker 400 NMR, Bruker 500 NMR, Varian 400-MR and VNMRS-500 spectrometers. ¹H NMR data are reported as follows: chemical shift (multiplicity (br=broad, s=singlet, d=doublet, t=triplet, q=quartet, p=pentet, sex=sextet, sept=septet and m=multiplet), integration, and assignment). Chemical shifts for ¹H NMR data are reported in ppm downfield from internal tetramethylsilane (TMS, b scale) using residual protons in the deuterated solvent as references. ¹³C NMR data were determined with ¹H decoupling, and the chemical shifts are reported downfield from tetramethylsilane (TMS, δ scale) in ppm versus the using residual carbons in the deuterated solvent as references.

### EXAMPLES

Actual examples are not indicated as such, whereas prophetic examples, if any, are marked prophetic. Syntheses of commercially available compounds are not shown. Unless otherwise noted the following conditions are used. Anhydrous toluene, hexanes, tetrahydrofuran, and diethyl ether were purified via passage through activated alumina and, in some cases, Q-5 reactant. Solvents used for experiments performed in a nitrogen-filled glovebox were further dried by storage over activated 3A molecular sieves. Glassware for moisture-sensitive reactions was dried in an oven overnight prior to use.

### Example 1: Synthesis of 3-bromo-2-hydroxythiophene (Example of Step A)

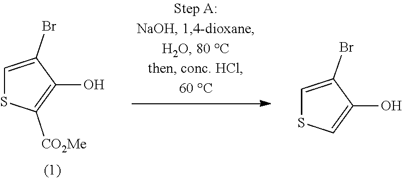 [SMILES: COC(=O)c1scc(Br)c1O ; Oc1cscc1Br]

To a suspension of 3-bromo-2-hydroxy-thiophene-1-carboxylic acid methyl ester (1) (10.020 grams, 42.267 mmol, 1.00 eq) in 1,4-dioxane (100 mL) and H₂O (450 mL) under nitrogen was added NaOH (50.000 g, 1.250 mol, 29.6 eq) all at once. The pale yellow mixture was equipped with a reflux condenser and placed in a mantle (i.e., heating mantle) heated to 80° C. After stirring (500 rpm) for 2.5 hours, thin layer chromatography (TLC) of the resulting golden yellow solution indicated complete conversion of (1) to a product having a lower Rf spot. The reaction mixture was removed from the mantle, allowed to gradually cool to 23° C., and placed in an ice water bath for 60 minutes. Then concentrated HCl (175 mL, 37%) was added over 10 minutes, and the resulting white heterogeneous mixture was removed from the ice water bath, placed in a mantle heated to 60° C., and stirred vigorously (1000 rpm) for 5 hours. The now pale golden yellow solution was removed from the mantle, allowed to cool gradually to 23° C., diluted with Et₂O (100 mL), stirred vigorously for 2 minutes, poured into a separatory funnel, partitioned, organics were washed with aqueous HCl (2×100 mL, 1 Normal (“N”)), residual organics were extracted from the aqueous layer using Et₂O (2×50 mL), dried over solid Na₂SO₄, decanted, and the Et₂O was removed via rotary evaporation to afford 3-bromo-2-hydroxythiophene as a solution in 1,4-dioxane (100 mL). The solution of 3-bromo-2-hydroxythiophene is used in step B without concentration or purification. An aliquot was removed, fully concentrated in vacuo, and NMR is consistent with pure 3-bromo-2-hydroxythiophene as a mixture of keto-enol tautomers where * indicates keto-enol tautomer: ¹H NMR (400 MHz, Chloroform-d) δ (8.34 (s, 1H)*), 7.12 (d, J=3.7 Hz, 1H), 6.43 (d, J=3.7 Hz, 1H), 5.49 (s, 1H), (3.72 (s, 2H)*). ¹C NMR (101 MHz, Chloroform-d) δ (210.23*), 195.46, 160.19, (149.69*), 121.43, (111.65*), (103.07*), 100.24, (37.05*).

### Example 2: Synthesis of 3-bromo-2-ethoxymethyloxythiophene (2) (Example of Step B)

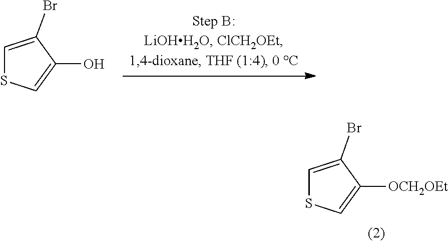

The solution of the 3-bromo-2-hydroxythiophene in 1,4-dioxane (100 mL) from Step A was diluted with non-anhydrous, non-deoxygenated THE (400 mL). Then H₂O (6 mL) was added. The solution was placed in an ice water bath, sparged with nitrogen for 1 hour, placed under a positive flow of nitrogen upon which solid lithium hydroxide-monohydrate (3.544 g, 84.453 mmol, 2.00 eq) was added. The now dark red-brown solution, was stirred vigorously (1000 rpm) for 1 hour, then neat chloromethyl ethyl ether (11.8 mL, 126.80 mmol, 3.00 eq, “ClCH₂OEt”) was added via syringe in a quick dropwise manner. After stirring for 2 hours at 0° C. the dark brown solution was diluted with aqueous NaOH (200 mL, 1 N), stirred for 2 minutes, THE was removed in vacuo, the biphasic mixture was diluted with CH₂Cl₂ (100 mL), suction filtered over a pad of diatomaceous earth, rinsed with CH₂Cl₂ (4×50 mL). The dark brown filtrate was poured into a separatory funnel, partitioned, organics were washed with aqueous NaOH (2×100 mL, 1 N), residual organics were extracted from the aqueous using CH₂Cl₂ (2×50 mL), combined, dried over solid Na₂SO₄, decanted, and carefully concentrated to afford a golden brown oil which was diluted with CH₂Cl₂ (25 mL), suction filtered over a pad of silica gel, rinsed with CH₂Cl₂ (4×50 mL), and the filtrate was concentrated to afford the 3-bromo-2-ethoxymethyloxythiophene (2) as a golden yellow oil (9.534 g, 40.209 mmol, 95% two steps). NMR is consistent with compound (2): ¹H NMR (400 MHz, Chloroform-d) δ 7.15 (d, J=3.6 Hz, 1H), 6.61 (d, J=3.5 Hz, 1H), 5.19 (s, 2H), 3.73 (q, J=7.1 Hz, 2H), 1.22 (t, J=7.1 Hz, 3H). ¹¹C NMR (101 MHz, Chloroform-d) δ 151.51, 121.50, 103.84, 101.55, 95.07, 64.53, 15.05.

### Example 3: Synthesis of 2-ethoxymethyloxy-3-(3′,6′-di-tert-butylcarbazolyl)lthiophene (4) (Example of Step C)

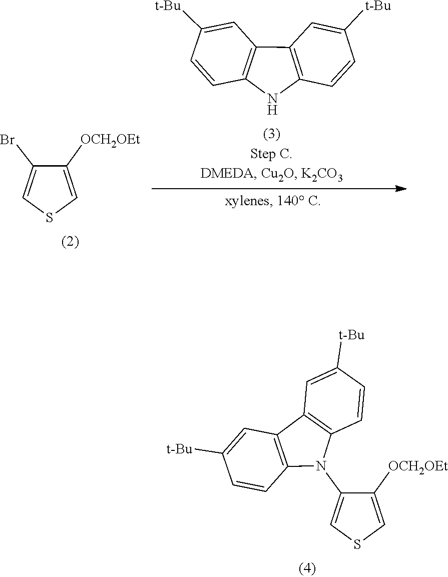

Step C. In a nitrogen filled continuous purge glovebox, a mixture of the bromothiophene (2) (5.883 g, 24.811 mmol, 1.00 eq), 3,6-di-t-butylcarbazole (15.252 g, 54.585 mmol, 2.20 eq), Cu₂O (7.100 g, 49.622 mmol, 2.00 eq), and K₂CO₃ (34.290 g, 248.11 mmol, 10.00 eq) was suspended in deoxygenated anhydrous xylenes (200 mL), N,N-DMEDA (10.7 mL, 99.244 mmol, 4.00 eq) was added, the mixture was equipped with a reflux condenser and a rubber septa, removed from the glovebox, placed under nitrogen, placed in a mantle heated to 140° C., stirred vigorously (1000 rpm) for 48 hours, removed from the mantle, the now deep red-black mixture was allowed to cool gradually to 23° C., CH₂Cl₂ (100 mL) was added, the mixture was stirred for 5 mins, suction filtered over a pad of silica gel, rinsed with CH₂Cl₂ (4×75 mL), the golden brown filtrate was concentrated to an amorphous solid, which was suspended in hexanes (50 mL), placed in a mantle heated to 60° C., after stirring (300 rpm) for 1 hr, the golden brown mixture was removed from the heating mantle, allowed to gradually cool to 23° C., the mixture was suction filtered, the residual white solid was rinsed with hexanes (4×20 mL), the golden brown filtrate solution was concentrated onto diatomaceous earth, and purified via silica gel chromatography using an ISCO chromatography purification system; 15% CH₂Cl₂ in hexanes to make the thiophene-carbazole (4) as a white amorphous foam (7.699 g, 17.673 mmol, 71%). NMR is consistent with compound (4). ¹H NMR (500 MHz, Chloroform-d) δ 8.12 (d, J=1.9 Hz, 2H), 7.45 (dd, J=8.6, 2.0 Hz, 2H), 7.32 (d, J=3.6 Hz, 1H), 7.20 (d, J=8.6 Hz, 2H), 6.89 (d, J=3.6 Hz, 1H), 3.56 (q, J=7.1 Hz, 2H), 1.47 (s, 18H), 1.16 (t, J=7.1 Hz, 3H). ¹³C NMR (126 MHz, Chloroform-d) δ 150.87, 142.60, 139.70, 127.62, 123.44, 123.08, 120.21, 116.07, 109.57, 102.36, 94.78, 64.37, 34.70, 32.03, 15.01.

### Example 4: Synthesis of 2-ethoxymethyloxy-3-(3′,6′-di-tert-butylcarbazolyl)-2-pinocolatoboryllthiophene (5) (Example of Step D)

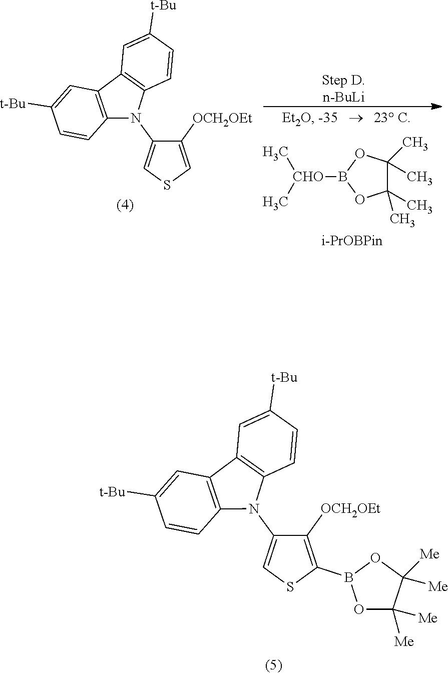

Step D. A golden yellow solution of the thiophene carbazole (4) (3.000 g, 6.887 mmol, 1.00 eq) in anhydrous deoxygenated Et₂O (75 mL) in a nitrogen filled continuous purge glovebox was placed in the freezer (−35° C.), and allowed to precool for 14 hours upon which a precooled solution of normal-butyl lithium (n-Butyllithium or n-BuLi) (3.50 mL, 8.608 mmol, 1.25 eq, titrated 2.5 M in hexanes) was added in a quick dropwise manner. The pale orange solution was allowed to sit in the freezer for 4 hours upon which the isopropoxyboropinacolate ester (“i-PrOBPin”) (2.81 mL, 13.774 mmol, 2.00 eq) was added neat. The now golden yellow solution was allowed to stir at 23° C. for 2 hours, the now white heterogeneous mixture was diluted with an aqueous phosphate buffer (20 mL, pH=8, 0.05 M), concentrated via rotary evaporation, the mixture was diluted with CH₂Cl₂ (25 mL) and water (25 mL), poured into a separatory funnel, partitioned, organics were washed with water (1×25 mL), residual organics were extracted with CH₂Cl₂ (2×25 mL), combined, dried over solid Na₂SO₄, decanted, concentrated, the resultant golden yellow foam was dissolved in CH₂Cl₂ (10 mL), suction filtered through a short pad of silica gel, rinsed with CH₂Cl₂ (4×20 mL), and the golden yellow filtrate solution was concentrated to afford the thiophene-boropinacolate ester (5) as a pale golden yellow foam (2.581 g, 4.596 mmol, 67%,). NMR was consistent with approximately 72% pure compound (5). ¹H NMR (500 MHz, Chloroform-d) δ 8.11-8.08 (m, 2H), 7.62 (d, J=0.9 Hz, 1H), 7.45 (dt, J=8.6, 1.4 Hz, 2H), 7.23 (dd, J=8.7, 0.7 Hz, 2H), 4.88 (d, J=0.8 Hz, 2H), 2.96-2.88 (m, 2H), 1.46 (s, 18H), 1.38 (s, 12H), 0.58 (t, J=7.1 Hz, 3H). ¹³C NMR (126 MHz, Chloroform-d) δ 158.93, 142.70, 139.53, 130.88, 127.58, 123.65, 123.00, 115.86, 109.77, 98.24, 84.20, 64.53, 34.71, 32.03, 24.80, 14.14. The 72% pure compound (5) was used in the subsequent reaction without further purification.

### Example 5: Synthesis of 2-iodo-4-(1′,1′,3′,3′-tetramethylbutyl)phenol

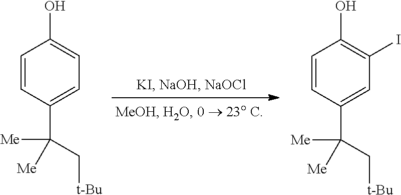 [SMILES: CC(C)(C)CC(C)(C)c1ccc(O)cc1 ; CC(C)(C)CC(C)(C)c1ccc(O)c(I)c1]

A clear colorless solution of 4-(1′,1′,3′,3′-tetramethylbutyl)phenol (3.324 g, 16.110 mmol, 1.00 eq), potassium iodide (KI, 3.477 g, 20.943 mmol, 1.30 eq), and aqueous NaOH (21 mL, 20.943 mmol, 1.30 eq, 1 N) in methanol (100 mL) and water (50 mL) under nitrogen was placed in an ice water bath and stirred vigorously for 1 hour, upon which precooled commercial aqueous bleach (26 mL, 20.943 mmol, 1.30 eq, 5.2% w/w) was added in a dropwise manner over 10 minutes. The now pale opaque yellow mixture was stirred for 2 hours at 0° C., the mixture was removed from the ice water bath, stirred at 23° C. for 3 hours, upon which solid NaH₂PO₄ (20 g) was added followed by a saturated aqueous mixture Na₂S₂O₃ (100 mL) to reduce residual iodine. Water (100 mL) was added to the mixture, which was stirred vigorously for 10 minutes, diluted with CH₂Cl₂ (50 mL), the biphasic yellow mixture was poured into a separatory funnel, partitioned, organics were washed with aqueous Na₂S₂O₃ (2×50 mL), residual organics were extracted from the aqueous layer using CH₂Cl₂ (2×50 mL), combined, dried over solid Na₂SO₄, decanted, and concentrated onto diatomaceous earth, and purified via silica gel chromatography; hexanes—10% CH₂Cl₂ in hexanes to give 2-iodo-4-(1′,1′,3′,3′-tetramethylbutyl)phenol as a clear colorless amorphous foam (3.240 g, 9.340 mmol, 58%). NMR is consistent with pure 2-iodo-4-(1′,1′,3′,3′-tetramethylbutyl)phenol: ¹H NMR (500 MHz, Chloroform-d) δ 7.60 (d, J=2.3 Hz, 1H), 7.24 (dd, J=8.5, 2.3 Hz, 1H), 6.90 (dd, J=8.6, 0.5 Hz, 1H), 5.11 (s, 1H), 1.68 (s, 2H), 1.32 (s, 6H), 0.73 (s, 9H). ¹³C NMR (126 MHz, Chloroform-d) δ 152.34, 144.65, 135.66, 128.14, 114.23, 85.38, 56.87, 37.93, 32.35, 31.81, 31.55.

### Example 6: Synthesis of 1,3-bis[2-iodo-4-(1′,1′,3′,3′-tetramethylbutyl)phenoxy]propane

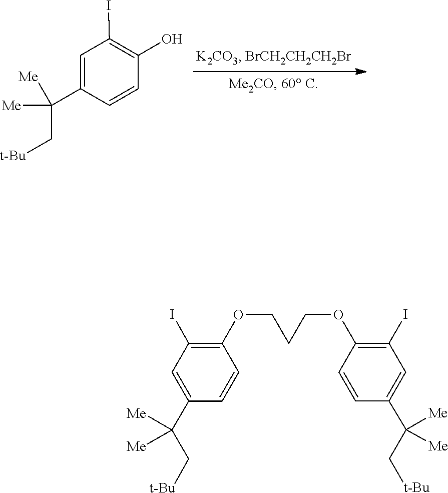 [SMILES: CC(C)(C)CC(C)(C)c1ccc(O)c(I)c1 ; CC(C)(C)CC(C)(C)c1ccc(OCCCOc2ccc(C(C)(C)CC(C)(C)C)cc2I)c(I)c1]

To a mixture of the iodophenol (2.345 g, 7.058 mmol, 2.00 eq) and K₂CO₃(2.926 g, 21.174 mmol, 7.00 eq) in acetone (30 mL) was added 1,3-dibromopropane (0.36 mL, 3.529 mmol, 1.00 eq), the mixture was equipped with a reflux condenser, placed under nitrogen, placed in a mantle heated to 60° C., and stirred (500 rpm) for 48 hours. The mixture was then removed from the mantle, allowed to cool to ambient temperature, diluted with CH₂Cl₂ (20 mL), the mixture was suction filtered through a pad of diatomaceous earth, rinsed with CH₂Cl₂ (4×20 mL), the pale golden brown filtrate solution was poured into a separatory funnel, washed with aqueous NaOH (2×20 mL, 1N), residual organics were extracted from the aqueous using CH₂Cl₂ (2×20 mL), dried over Na₂SO₄, decanted, the pale golden brown solution was concentrated onto diatomaceous earth, and purified via silica gel chromatography using an ISCO; hexanes—30% CH₂Cl₂ in hexanes to afford the bisiodide as a white solid (2.091 g, 2.968 mmol, 84%). NMR indicated product. ¹H NMR (500 MHz, cdcl₃) 6 7.72 (d, J=2.3 Hz, 2H), 7.28-7.25 (m, 2H), 6.79 (d, J=8.6 Hz, 2H), 4.26 (t, J=6.0 Hz, 4H), 2.33 (p, J=6.0 Hz, 2H), 1.68 (s, 4H), 1.32 (s, 12H), 0.72 (s, 18H). ¹³C NMR (126 MHz, cdcl₃) δ 154.98, 144.70, 137.15, 127.12, 111.51, 86.30, 65.70, 56.87, 37.91, 32.35, 31.83, 31.56, 29.26.

### Example 7: Synthesis of 4-octyloxyphenol

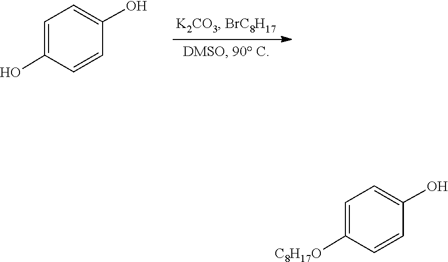

To a mixture of hydroquinone (20.000 g, 181.64 mmol, 1.00 eq) and K₂CO₃ (100.40 g, 726.56 mmol, 4.00 eq) in DMSO (600 mL) was added 1-bromooctane (40.8 mL, 236.13 mmol, 1.30 eq). The mixture was placed under nitrogen, placed in a mantle heated to 90° C., stirred (500 rpm) for 36 hours, removed from the mantle, allowed to cool to ambient temperature, water (200 mL) and KH₂PO₄ (100 grams) were added, and stirred for approximately 10 mins. Then, EtOAc/hexanes (200 mL, 1:1) was added, the biphasic mixture was poured into a separatory funnel, partitioned, organics were washed with water (3×100 mL), residual organics were extracted from the aqueous using EtOAc/hexanes (2×100 mL, 1:1), combined, dried over Na₂SO₄, decanted, concentrated onto diatomaceous earth, and purified via silica gel chromatography using an ISCO; 40%-80% CH₂Cl₂ in hexanes to afford 4-octylether phenol as a pale golden brown solid (16.646 g, 74.871 mmol, 41%). NMR indicated product. ¹H NMR (400 MHz, CDCl₃) 56.84-6.73 (m, 4H), 5.11 (s, 1H), 3.92 (t, J=6.6 Hz, 2H), 1.78 (p, J=6.8 Hz, 2H), 1.46 (p, J=7.0 Hz, 2H), 1.34 (ddd, J=19.4, 10.1, 5.1 Hz, 8H), 0.99-0.84 (m, 3H). ¹³C NMR (101 MHz, CDCl₃) δ 153.24, 149.44, 116.07, 115.74, 68.91, 31.83, 29.38, 29.26, 26.06, 22.67, 14.11.

### Example 8: Synthesis of 4-octyloxyphenolmethyl ethyl ether

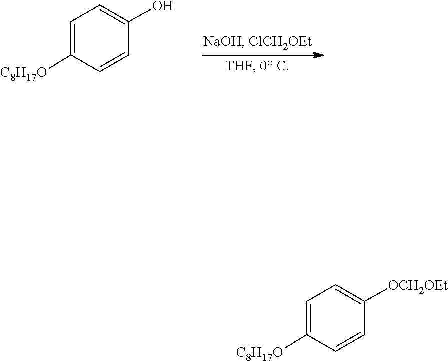

A clear, colorless solution of the iodo-phenol (18.405 g, 0.08278 mol, 1.00 eq) in THF (500 mL) was sparged under positive flow of nitrogen for 15 mins upon which an aqueous solution of NaOH (13.2 mL, 0.500 mol, 6.00 eq, 50% w/w) was added via syringe in a quick dropwise manner. After stirring (500 rpm) for 60 mins at 23° C., neat chloromethyl ethyl ether (23.0 mL, 0.24835 mol, 3.00 eq) was added via syringe in a quick dropwise manner to the clear pale yellow solution. After stirring for 3 hours at 23° C., the now white heterogeneous mixture was diluted with aqueous NaOH (100 mL, 1 N), THF was removed via rotary evaporation, the resultant white biphasic mixture was diluted with CH₂Cl₂ (100 mL), poured into a separatory funnel, partitioned, organics were washed with aqueous NaOH (2×100 mL, 1 N), residual organics were extracted from the aqueous using CH₂Cl₂ (2×50 mL), combined, dried over solid Na₂SO₄, decanted, and concentrated. The resultant pale yellow oil was diluted in CH₂Cl₂ (20 mL), suction filtered through a pad of silica gel, rinsed with CH₂Cl₂ (4×25 mL), and the filtrate was concentrated to afford the phenolic methyl ethyl ether as a clear amber oil (23.116 g, 0.08244 mol, 99%). NMR indicated product. ¹H NMR (400 MHz, cdcl₃) δ 7.00-6.92 (m, 2H), 6.84-6.76 (m, 2H), 5.13 (s, 2H), 3.88 (t, J=6.6 Hz, 2H), 3.71 (q, J=7.1 Hz, 2H), 1.74 (dt, J=14.6, 6.7 Hz, 2H), 1.43 (dq, J=10.2, 6.0 Hz, 2H), 1.37-1.24 (m, 8H), 1.21 (t, J=7.1 Hz, 3H), 0.92-0.83 (m, 3H). ¹³C NMR (101 MHz, cdcl₃) 6 154.19, 151.31, 117.50, 115.26, 94.00, 68.51, 63.99, 31.79, 29.35, 29.22, 26.03, 22.63, 15.09, 14.07.

### Example 9: Synthesis of 2-iodo-4-octyloxyphenolmethyl ethyl ether

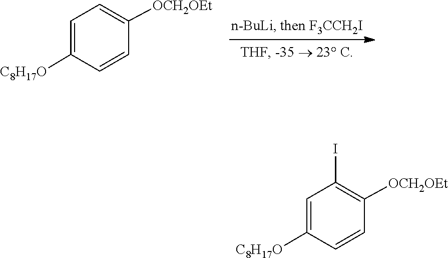

Prior to use, the bisether was azeotropically dried using toluene (4×10 mL). A clear, colorless solution of the bisether (4.359 g, 15.545 mmol, 1.00 eq) in anhydrous deoxygenated THF (150 mL) in a continuous purge nitrogen filled glovebox was placed in a freezer (−35° C.) for 16 hours. n-BuLi (10.9 mL, 27.204 mmol, 1.75 eq, 2.5 M in hexanes) was added, the dark amber solution was allowed to sit in the freezer for 20 hours, 2-iodo-1,1,1-trifluoroethane (3.8 mL, 38.863 mmol, 2.50 eq) was added neat in a quick dropwise manner, the now golden brown solution was allowed to remain in the freezer for 30 mins, removed, stirred (500 rpm) for 4 hours at 23° C., the mixture was removed from the glovebox, neutralized with H₂O (50 mL), and THE was removed via rotary evaporation. The brown mixture was diluted with CH₂Cl₂ (50 mL) and water (50 mL), poured into a separatory funnel, partitioned, organics were extracted with CH₂Cl₂ (2×50 mL), combined, dried over Na₂SO₄, decanted, concentrated onto diatomaceous earth, and purified by silica gel chromatography using an ISCO; hexanes—20% CH₂Cl₂ in hexanes to afford the iodide as a clear colorless oil (5.208 g, 12.818 mmol, 82%). NMR indicated product. ¹H NMR (500 MHz, cdcl₃) 6 7.31 (d, J=2.9 Hz, 1H), 7.00 (d, J=8.9 Hz, 1H), 6.83 (dd, J=9.0, 2.9 Hz, 1H), 5.19 (s, 2H), 3.88 (t, J=6.6 Hz, 2H), 3.78 (q, J=7.1 Hz, 2H), 1.74 (dq, J=8.9, 6.6 Hz, 2H), 1.47-1.39 (m, 2H), 1.39-1.28 (m, 8H), 1.23 (d, J=7.1 Hz, 3H), 0.91-0.85 (m, 3H). ¹³C NMR (126 MHz, cdcl₃) δ 154.71, 150.48, 125.02, 116.25, 115.55, 94.60, 87.65, 68.74, 64.55, 31.80, 29.32, 29.24, 29.22, 25.99, 22.65, 15.07, 14.09.

### Example 10: Synthesis of 2-iodo-4-octyloxyphenol

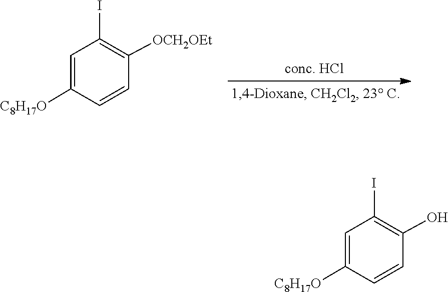

To a clear pale yellow solution of the iodo-phenol (17.510 g, 0.04310 mol, 1.00 eq) in 1,4-dioxane (50 mL) and CH₂Cl₂ (50 mL) under nitrogen at 23° C. was added conc. HCl (25 mL). After stirring (500 rpm) for 8 hours at 23° C., the now golden brown mixture was diluted with water (100 mL) and CH₂Cl₂ (50 mL), poured into a separatory funnel, partitioned, organics were extracted from the aqueous using CH₂Cl₂ (2×25 mL), combined, dried over Na₂SO₄, decanted, concentrated, CH₂Cl₂ (20 mL) was added, the dark brown solution was suction filtered over a pad of silica gel, rinsed with CH₂Cl₂ (4×25 mL), and the filtrate was concentrated to afford the iodophenol as a clear amber oil (14.810 g, 0.04253 mol, 99%). NMR indicated product with minor impurities, which was used in the subsequent reaction without further purification. ¹H NMR (400 MHz, cdcl₃) 6 7.17 (d, J=2.8 Hz, 1H), 6.88 (d, J=8.9 Hz, 1H), 6.80 (dd, J=8.9, 2.9 Hz, 1H), 4.93 (s, 1H), 3.85 (t, J=6.5 Hz, 2H), 1.81-1.65 (m, 2H), 1.41 (p, J=6.9 Hz, 2H), 1.37-1.20 (m, 8H), 0.96-0.79 (m, 3H). ¹³C NMR (101 MHz, cdcl₃) δ 153.51, 149.03, 123.45, 116.95, 115.04, 85.07, 68.94, 31.79, 29.32, 29.24, 29.21, 25.98, 22.64, 14.09.

### Example 11: Synthesis of 1,3bis-[2-iodo-4-(octyloxy)phenoxy]propane

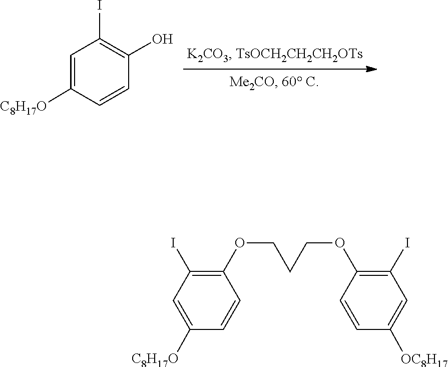

A mixture of 2-iodo-4-octylether phenol (17.800 g, 51.116 mmol, 2.20 eq), K₂CO₃ (22.478 g, 162.65 mmol, 7.00 eq), and the bistosylate (8.933 g, 23.235 mmol, 1.00 eq) in acetone (200 mL) was equipped with a reflux condenser, placed under nitrogen, placed in a mantle heated to 60° C., stirred (500 rpm) for 36 hours, removed from the mantle, allowed to cool to ambient temperature, diluted with CH₂Cl₂ (100 mL), aqueous NaOH (100 mL, 1 N) was added, the biphasic mixture was poured into a separatory funnel, partitioned, organics were washed with aqueous NaOH (2×50 mL, 1N), residual organics were extracted from the aqueous using CH₂Cl₂ (2×50 mL), dried over Na₂SO₄, decanted, the golden brown solution was concentrated onto diatomaceous earth, and purified via silica gel chromatography using an ISCO; hexanes—50% CH₂Cl₂ in hexanes to afford the bisiodide as a white solid (13.700 g, 18.601 mmol, 80%). NMR indicated product. ¹H NMR (500 MHz, cdcl₃) δ 7.32 (d, J=2.8 Hz, 2H), 6.84 (dd, J=8.9, 2.8 Hz, 2H), 6.80 (d, J=8.9 Hz, 2H), 4.21 (t, J=6.0 Hz, 4H), 3.87 (t, J=6.6 Hz, 4H), 2.29 (p, J=5.9 Hz, 2H), 1.78-1.69 (m, 4H), 1.43 (tt, J=9.2, 3.7 Hz, 4H), 1.32 (dddd, J=17.9, 10.7, 5.3, 1.5 Hz, 16H), 0.94-0.84 (m, 6H). ¹³C NMR (126 MHz, cdcl₃) δ 153.96, 151.82, 125.31, 115.46, 113.29, 86.95, 68.83, 66.57, 31.81, 29.39, 29.34, 29.28, 29.24, 26.00, 22.66, 14.12.

### Example 12: Synthesis of 2-iodo-4-(triethylmethyl)phenol

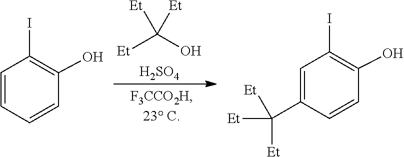

To a golden yellow suspension of the starting 2-iodophenol (2.000 g, 9.091 mmol, 1.00 eq) in trifluoroacetic acid (5 mL) under nitrogen at 23° C. was added neat 3-ethyl-3-pentanol (1.54 mL, 10.909 mmol, 1.20 eq) followed by the dropwise addition of neat concentrated H₂SO₄ (0.14 mL, 2.612 mmol, 0.29 eq). After stirring (500 rpm) for 16 hours at 23° C., the mixture was poured into ice water (100 mL), methylene chloride was added (25 mL), the biphasic mixture was poured into a separatory funnel, partitioned, organics were washed with water (1×25 mL), and then a saturated aqueous mixture of NaHCO₃ (2×25 mL). Residual organics were extracted from the aqueous layer using CH₂Cl₂ (1×20 mL), combined, dried over solid Na₂SO₄, decanted, and concentrated onto diatomaceous earth, and purified via silica gel chromatography; hexanes—15% CH₂Cl₂ to afford the 2-iodo-4-(triethylmethyl)phenol as a clear colorless amorphous foam (1.246 g, 3.916 mmol, 43%). NMR is consistent with pure 2-iodo-4-(triethylmethyl)phenol. ¹H NMR (400 MHz, cdcl₃) δ 7.50 (d, J=2.3 Hz, 1H), 7.15 (dd, J=8.6, 2.3 Hz, 1H), 6.91 (d, J=8.6 Hz, 1H), 5.07 (s, 1H), 1.58 (q, J=7.4 Hz, 6H), 0.63 (t, J=7.3 Hz, 9H).

### Example 13: Synthesis of 1,3-bis(2-iodo-4-triethylmethylphenoxy)propane

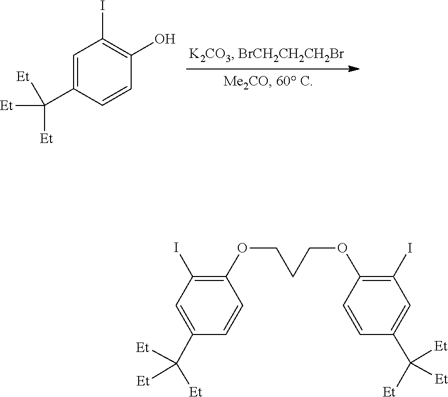 [SMILES: CCC(CC)(CC)c1ccc(O)c(I)c1 ; CCC(CC)(CC)c1ccc(OCCCOc2ccc(C(CC)(CC)CC)cc2I)c(I)c1]

To a mixture of the iodophenol (4.528 g, 14.230 mmol, 2.10 eq) and K₂CO₃(7.867 g, 56.920 mmol, 8.40 eq) in acetone (100 mL) was added 1,3-dibromopropane (0.69 mL, 6.776 mmol, 1.00 eq), the mixture was equipped with a reflux condenser, placed under nitrogen, placed in a mantle heated to 60° C., and stirred (500 rpm) for 48 hours. The mixture was then removed from the mantle, allowed to cool to ambient temperature, diluted with CH₂Cl₂ (20 mL), the mixture was suction filtered through a pad of diatomaceous earth, rinsed with CH₂Cl₂ (4×20 mL), the pale golden brown filtrate solution was poured into a separatory funnel, washed with aqueous NaOH (2×20 mL, 1N), residual organics were extracted from the aqueous using CH₂Cl₂ (2×20 mL), dried over Na₂SO₄, decanted, the pale golden brown solution was concentrated onto diatomaceous earth, and purified via silica gel chromatography using an ISCO; hexanes—30% CH₂Cl₂ in hexanes to afford the bisiodide as a white solid (4.125 g, 6.098 mmol, 90%). NMR indicated product. ¹H NMR (500 MHz, cdcl₃) δ 7.65 (d, J=2.3 Hz, 2H), 7.20 (dd, J=8.6, 2.3 Hz, 2H), 6.81 (d, J=8.6 Hz, 2H), 4.27 (t, J=6.0 Hz, 4H), 2.34 (p, J=6.0 Hz, 2H), 1.61 (q, J=7.4 Hz, 12H), 0.64 (t, J=7.4 Hz, 18H). ¹³C NMR (126 MHz, cdcl₃) δ 154.84, 141.84, 137.82, 127.94, 111.46, 86.58, 65.60, 43.14, 29.30, 28.69, 7.93.

### Example 14: Synthesis of 2-iodo-4-(3′,5′-di-t-butylphenyl)phenol

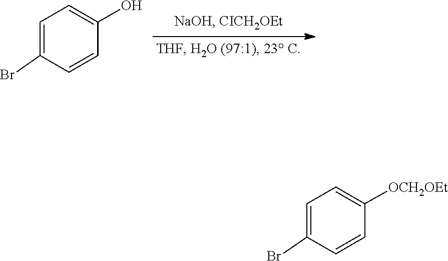

A clear, colorless solution of the phenol (5.000 g, 28.900 mmol, 1.00 eq) in THE (200 mL) and H₂O (2 mL) was sparged under positive flow of nitrogen for 1 hr upon which an aqueous solution of NaOH (4.6 mL, 57.800 mmol, 2.00 eq, 50% w/w) was added via syringe in a quick dropwise manner. After stirring (500 rpm) for 30 mins at 23° C., neat chloromethyl ethyl ether (8.0 mL, 86.700 mmol, 3.00 eq) was added via syringe in a quick dropwise manner to the clear colorless solution. After stirring for 2 hours at 23° C., the now white heterogeneous mixture was diluted with aqueous NaOH (100 mL, 1 N), THE was removed via rotary evaporation, the resultant white biphasic mixture was diluted with CH₂Cl₂ (100 mL), poured into a separatory funnel, partitioned, organics were washed with aqueous NaOH (2×50 mL, 1 N), residual organics were extracted from the aqueous (2×25 mL), combined, dried over solid Na₂SO₄, decanted, and concentrated. The resultant pale yellow oil was diluted in CH₂Cl₂ (10 mL), suction filtered through a silica gel pad, rinsed with CH₂Cl₂ (4×25 mL), and the filtrate was concentrated to afford the phenolic methyl ethyl ether as a clear colorless oil (6.628 g, 28.681 mmol, 99%). NMR indicated pure product. ¹H NMR (400 MHz, Chloroform-d) δ 7.43-7.31 (m, 2H), 6.97-6.86 (m, 2H), 5.17 (s, 2H), 3.70 (qd, J=7.1, 1.2 Hz, 2H), 1.20 (td, J=7.1, 1.2 Hz, 3H). ¹³C NMR (101 MHz, Chloroform-d) δ 156.48, 132.26, 118.04, 114.01, 93.19, 64.31, 15.07.

### Example 15: Synthesis of 4-(3,5-di-t-butylphenyl)phenoxymethyl ethyl ether

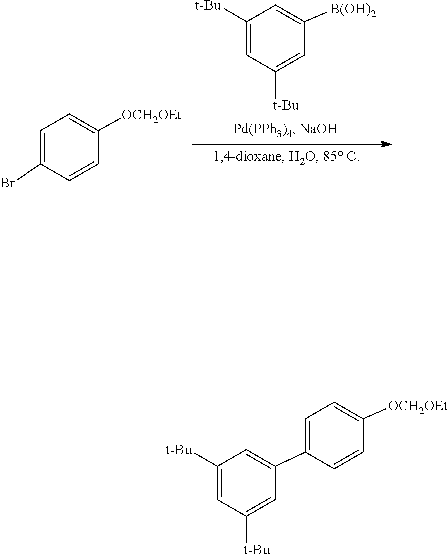

A mixture of boronic acid (2.500 g, 10.677 mmol, 1.50 eq), the aryl bromide (1.645 g, 7.118 mmol, 1.00 eq), NaOH (1.281 g, 32.031 mmol, 4.50 eq), and Pd(PPh₃)₄(0.823 g, 0.7118 mmol, 0.10 eq) in a flask equipped with a reflux condenser was evacuated, back-filled with nitrogen, and the evacuation/refill process was repeated 3x more. Freshly sparged 1,4-dioxane (75 mL) and H₂O (15 mL) were added, the mixture was placed in a mantle heated to 85° C., stirred (500 rpm) for 40 hours, removed from the mantle, allowed to cool to ambient temperature, diluted with CH₂Cl₂ (20 mL), suction filtered through a pad of silica gel, rinsed with CH₂Cl₂ (4×20 mL), the filtrate was concentrated onto diatomaceous earth, and purified via silica gel chromatography using an ISCO; hexanes—50% CH₂Cl₂ in hexanes to afford the biaryl protected phenol as a clear colorless amorphous foam (2.078 g, 6.103 mmol, 86%). NMR indicated product. ¹H NMR (400 MHz, cdcl₃) δ 7.54-7.49 (m, 2H), 7.40 (d, J=1.8 Hz, 1H), 7.38 (d, J=1.8 Hz, 2H), 7.15-7.08 (m, 2H), 5.27 (s, 2H), 3.77 (q, J=7.1 Hz, 2H), 1.38 (s, 18H), 1.25 (t, J=7.1 Hz, 3H). ¹³C NMR (101 MHz, cdcl₃) δ 156.72, 151.02, 140.23, 136.17, 128.43, 121.42, 120.95, 116.39, 93.24, 64.23, 34.94, 31.52, 15.12.

### Example 16: Synthesis of 2-iodo-4-(3,5-di-t-butylphenyl)phenoxymethyl ethyl ether

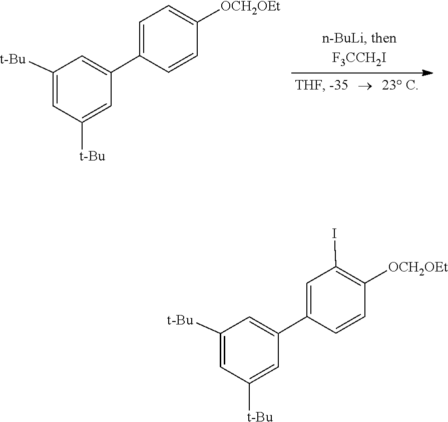

Prior to use, the ether was azeotropically dried using toluene (4×10 mL). A clear, colorless solution of the ether (2.078 g, 6.103 mmol, 1.00 eq) in anhydrous deoxygenated THE (50 mL) in a continuous purge nitrogen filled glovebox was placed in a freezer (−35° C.) for 16 hours. n-BuLi (3.20 mL, 7.934 mmol, 1.30 eq, 2.5 M in hexanes) was added, the amber solution was allowed to sit in the freezer for 12 hours, then 2-iodo-1,1,1-trifluoroethane (0.90 mL, 9.155 mmol, 1.50 eq) was added neat in a quick dropwise manner, the now golden brown solution was allowed to remain in the freezer for 60 mins, removed, stirred (500 rpm) for 2 hours at 23° C., the mixture was removed from the glovebox, neutralized with H₂O (50 mL), and THE was removed via rotary evaporation. The golden brown mixture was diluted with CH₂Cl₂ (50 mL) and water (50 mL), poured into a separatory funnel, partitioned, organics were extracted with CH₂Cl₂ (2×25 mL), combined, dried over Na₂SO₄, decanted, concentrated onto diatomaceous earth, and purified by silica gel chromatography using an ISCO; 5%-75% CH₂Cl₂ in hexanes to afford the iodide as a white foam (2.783 g, 5.967 mmol, 98%). NMR indicated product. ¹H NMR (400 MHz, cdcl₃) δ 7.97 (d, J=2.2 Hz, 1H), 7.49 (dd, J=8.5, 2.2 Hz, 1H), 7.42 (t, J=1.8 Hz, 1H), 7.32 (d, J=1.8 Hz, 2H), 7.15 (d, J=8.5 Hz, 1H), 5.32 (s, 2H), 3.80 (q, J=7.1 Hz, 2H), 1.37 (s, 18H), 1.25 (t, J=7.1 Hz, 3H). ¹³C NMR (101 MHz, cdcl₃) δ 155.39, 151.22, 138.82, 138.20, 138.13, 128.47, 121.43, 121.38, 114.88, 93.78, 87.51, 64.71, 34.96, 31.51, 15.08.

### Example 17: Synthesis of 2-iodo-4-(3,5-di-t-butylphenyl)phenol

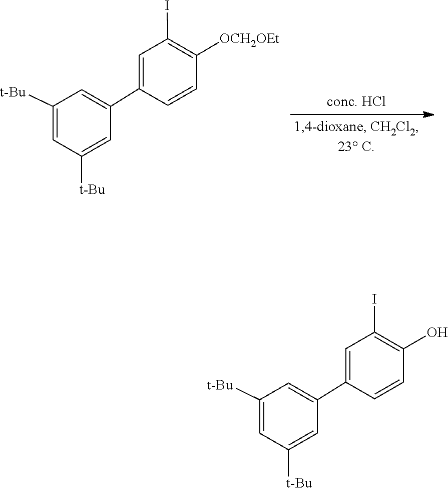

To a clear, colorless solution of the ether (2.783 g) in 1,4-dioxane (25 mL) and CH₂Cl₂ (25 mL) under nitrogen at 23° C. was added conc. HCl (15 mL), the mixture was stirred (300 rpm) for 16 hours, water (30 mL) and CH₂Cl₂ (30 mL) was added to the golden brown mixture, which was poured into a separatory funnel, partitioned, organics were extracted with CH₂Cl₂ (2×25 mL), combined, dried over Na₂SO₄, decanted, concentrated onto diatomaceous earth, and purified via silica gel chromatography using an ISCO; hexanes—50% CH₂Cl₂ in hexanes to afford the iodophenol as a white foam (2.334 g, 5.716 mmol, 96%). NMR indicated product.

¹H NMR (500 MHz, cdcl₃) δ 7.88 (d, J=2.1 Hz, 1H), 7.50 (dd, J=8.4, 2.1 Hz, 1H), 7.44 (t, J=1.8 Hz, 1H), 7.34 (d, J=1.8 Hz, 2H), 7.08 (d, J=8.4 Hz, 1H), 5.34 (s, 1H), 1.40 (s, 18H). 13C NMR (126 MHz, cdcl₃) δ 154.04, 151.29, 138.76, 137.25, 136.85, 129.38, 121.44, 121.31, 115.10, 86.12, 35.00, 31.55.

### Example 18: Synthesis of 1,3-bis(2-iodo-4-(3′,5′-di-t-butylphenyl)phenoxy)propane

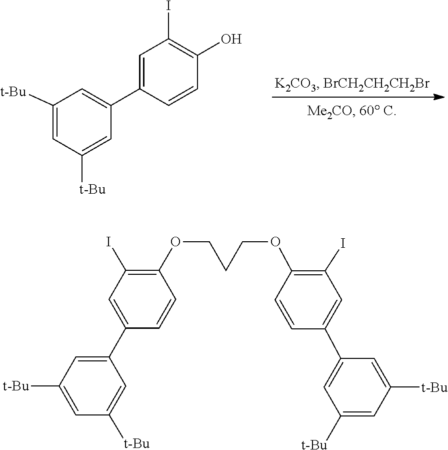 [SMILES: CC(C)(C)c1cc(-c2ccc(O)c(I)c2)cc(C(C)(C)C)c1 ; CC(C)(C)c1cc(-c2ccc(OCCCOc3ccc(-c4cc(C(C)(C)C)cc(C(C)(C)C)c4)cc3I)c(I)c2)cc(C(C)(C)C)c1]

To a mixture of the iodophenol (1.000 g, 2.449 mmol, 2.10 eq) and K₂CO₃(1.128 g, 8.162 mmol, 7.00 eq) in acetone (30 mL) was added 1,3-dibromopropane (0.12 mL, 1.166 mmol, 1.00 eq), the mixture was equipped with a reflux condenser, placed under nitrogen, placed in a mantle heated to 60° C., and stirred (500 rpm) for 48 hours. The mixture was then removed from the mantle, allowed to cool to ambient temperature, diluted with CH₂Cl₂ (20 mL), the mixture was suction filtered through a pad of diatomaceous earth, rinsed with CH₂Cl₂ (4×20 mL), the pale golden brown filtrate solution was poured into a separatory funnel, washed with aqueous NaOH (2×20 mL, 1N), residual organics were extracted from the aqueous using CH₂Cl₂ (2×20 mL), dried over Na₂SO₄, decanted, the pale golden brown solution was concentrated onto diatomaceous earth, and purified via silica gel chromatography using an ISCO; hexanes—30% CH₂Cl₂ in hexanes to afford the bisiodide as a white solid (0.844 g, 0.9852 mmol, 84%). NMR indicated product. ¹H NMR (500 MHz, cdcl₃) b 7.98 (d, J=2.2 Hz, 1H), 7.53 (dd, J=8.5, 2.2 Hz, 1H), 7.42 (t, J=1.8 Hz, 1H), 7.33 (d, J=1.8 Hz, 2H), 6.97 (d, J=8.5 Hz, 1H), 4.38 (t, J=5.9 Hz, 2H), 2.42 (p, J=5.9 Hz, 1H), 1.38 (s, 18H). ¹³C NMR (126 MHz, cdcl₃) δ 156.57, 151.22, 138.86, 138.12, 137.28, 128.51, 121.36, 121.33, 112.25, 87.08, 65.80, 34.97, 31.53, 29.19.

### Example 19: Synthesis of 2-iodo-4-(dimethyl-octyl-silyl)phenoxymethyl ethyl ether

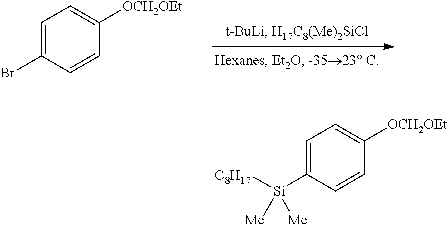

Prior to the experiment, the starting protected phenol was azeotropically dried using anhydrous toluene (4×10 mL). In a continuous purge, nitrogen filled glovebox, a clear colorless solution of the protected phenol (4.042 g, 17.491 mmol, 1.00 eq) in anhydrous deoxygenated hexanes (100 mL) was placed in a freezer cooled to −35° C. for 16 hours, upon which a solution of t-BuLi (26.0 mL, 43.728 mmol, 2.50 eq, 1.6 M in pentane) was added via syringe in a quick dropwise manner. Then, precooled anhydrous, deoxygenated Et₂O (5 mL) was added dropwise to the now pale yellow solution. The solution immediately became a pale yellow heterogeneous mixture, which was allowed to remain the freezer for 1 hr, upon which neat chlorodimethyloctylsilane (7.30 mL, 30.609 mmol, 1.75 eq) was added via syringe in a quick dropwise manner. The now golden yellow mixture was stirred (300 rpm) at 23° C. in the glovebox for 3 hours, removed from the glovebox, diluted with an aqueous saturated mixture of NaHCO₃ (50 mL) and Et₂O (50 mL), poured into a separatory funnel, partitioned, residual organics were washed with an aqueous saturated mixture of NaHCO₃ (2×25 mL), residual organics were extracted from the aqueous using Et₂O (2×25 mL), combined, dried over solid Na₂SO₄, suction filtered through silica gel, rinsed with Et₂O (3×25 mL), and the clear pale yellow solution was concentrated to afford the protected phenol as a clear pale yellow oil (4.498 g, 13.945 mmol, 80%). NMR indicated product. The material was used without further purification. ¹H NMR (400 MHz, cdcl₃) δ 7.44-7.38 (m, 2H), 7.05-6.99 (m, 2H), 5.22 (s, 2H), 3.72 (q, J=7.1 Hz, 2H), 1.33-1.18 (m, 15H), 0.90-0.83 (m, 3H), 0.70 (t, J=7.7 Hz, 2H), 0.21 (s, 6H).

### Example 20: Synthesis of 2-iodo-4-(dimethyl-octyl-silyl)phenoxymethyl ethyl ether

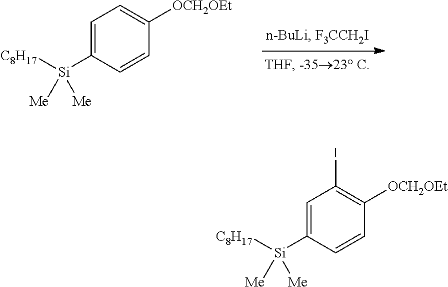

Prior to the experiment, the starting protected phenol was azeotropically dried using anhydrous toluene (4×10 mL). In a continuous purge, nitrogen filled glovebox, a clear colorless solution of the protected phenol (4.498 g, 13.945 mmol, 1.00 eq) in anhydrous deoxygenated THE (100 mL) was placed in a freezer cooled to −35° C. for 16 hours, upon which a solution of n-BuLi (8.40 mL, 20.917 mmol, 1.50 eq, 2.5 M in hexanes) was added via syringe in a quick dropwise manner, the now pale golden yellow solution remained in the freezer for 12 hours, then F₃CCH₂I (2.40 mL, 24.404 mmol, 1.75 eq) was added via syringe in a quick dropwise manner. The now golden yellow mixture was stirred (300 rpm) at 23° C. in the glovebox for 3 hours, removed from the glovebox, diluted with water (50 mL), THE was removed via rotary evaporation, the mixture was diluted with CH₂Cl₂ (50 mL), poured into a separatory funnel, partitioned, residual organics were extracted from the aqueous using CH₂Cl₂ (2×25 mL), combined, dried over solid Na₂SO₄, decanted, concentrated onto Diatomaceous earth, and purified via silica gel chromatography using the ISCO; hexanes −25% CH₂Cl₂ in hexanes to afford the iodide as a clear colorless oil (5.630 g, 12.551 mmol, 90%). NMR indicated product. ¹H NMR (500 MHz, cdcl₃) δ 7.86 (d, J=1.5 Hz, 1H), 7.38 (dd, J=8.1, 1.5 Hz, 1H), 7.08 (d, J=8.1 Hz, 1H), 5.29 (s, 2H), 3.77 (q, J=7.1 Hz, 2H), 1.32-1.19 (m, 15H), 0.91-0.85 (m, 3H), 0.74-0.68 (m, 2H), 0.22 (s, 6H). ¹³C NMR (126 MHz, cdcl₃) δ 156.53, 144.47, 135.17, 134.85, 114.55, 93.47, 88.00, 64.69, 33.53, 31.90, 29.24, 23.77, 22.66, 15.68, 15.04, 14.11, -2.93.

### Example 21: Synthesis of 2-iodo-4-(dimethyl-octyl-silyl)phenol

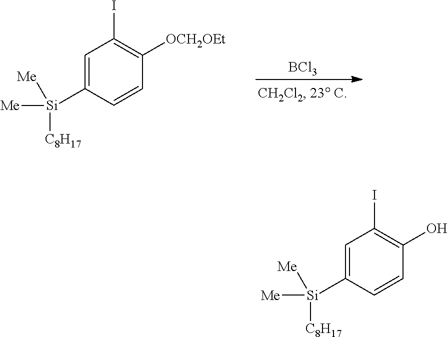

To a clear pale yellow solution of the protected phenol (0.600 g, 1.338 mmol, 1.00 eq) in anhydrous CH₂Cl₂ (20 mL) under nitrogen at 23° C. was added a solution of BCl₃ (2.70 mL, 2.676 mmol, 2.00 eq, 1 M in CH₂Cl₂). After stirring (500 rpm) for 2 hours at 23° C., an aqueous phosphate buffer (10 mL, pH=4) was added, followed by an aqueous solution of H₃PO₄ (2.5 mL, 85% in water) and water (2.5 mL), was added, the golden brown mixture was stirred for 1 hr, diluted with CH₂Cl₂ (20 mL) and water (20 mL), poured into a separatory funnel, partitioned, organics were extracted from the aqueous layer using CH₂Cl₂ (2×20 mL), combined, dried over Na₂SO₄, decanted, concentrated, CH₂Cl₂ (10 mL) was then added to the golden brown oil, the brown solution was suction filtered over a pad of silica gel, rinsed with CH₂Cl₂ (4×20 mL), and concentrated to afford the phenol as a clear pale yellow oil (0.510 g, 1.306 mmol, 98%). NMR indicated product. ¹H NMR (500 MHz, cdcl₃) δ 7.73 (d, J=1.4 Hz, 1H), 7.34 (dd, J=7.9, 1.5 Hz, 1H), 6.98 (d, J=7.9 Hz, 1H), 5.29 (s, 1H), 1.33-1.20 (m, 12H), 0.87 (t, J=7.0 Hz, 3H), 0.70 (t, J=8.0 Hz, 2H), 0.22 (s, 6H). ¹³C NMR (126 MHz, cdcl₃) δ 155.10, 143.36, 135.58, 134.13, 115.03, 86.99, 33.51, 31.91, 29.23, 23.76, 22.66, 15.71, 14.11, −2.91.

### Example 22: Synthesis of 1,3-bis[2-iodo-4-(dimethyl-octyl-silyl)phenoxy]propane

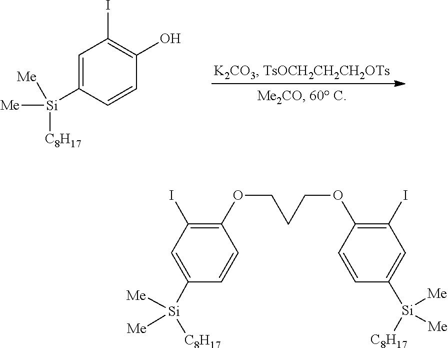

A mixture of 2-iodo-4-dimethyloctylsilyl phenol (0.500 g, 1.281 mmol, 2.20 eq), K₂CO₃ (0.563 g, 4.076 mmol, 7.00 eq), and the bistosylate (0.224 g, 0.5823 mmol, 1.00 eq) in acetone (20 mL) was equipped with a reflux condenser, placed under nitrogen, placed in a mantle heated to 60° C., stirred (500 rpm) for 36 hours, removed from the mantle, allowed to cool to ambient temperature, diluted with CH₂Cl₂ (20 mL), aqueous NaOH (20 mL, 1N) was added, the biphasic mixture was poured into a separatory funnel, partitioned, organics were washed with aqueous NaOH (2×20 mL, 1 N), residual organics were extracted from the aqueous using CH₂Cl₂ (2×20 mL), dried over Na₂SO₄, decanted, the pale golden brown solution, concentrated, CH₂Cl₂ (10 mL) was added, suction filtered through silica gel, rinsed with CH₂Cl₂ (4×20 mL), and the filtrate was concentrated to afford the bisiodide as a clear colorless oil (0.460 g, 0.5604 mmol, 96%). NMR indicated product. ¹H NMR (400 MHz, cdcl₃) δ 7.82 (d, J=1.5 Hz, 2H), 7.37 (dd, J=8.0, 1.5 Hz, 2H), 6.85 (d, J=8.0 Hz, 2H), 4.28 (t, J=5.9 Hz, 4H), 2.34 (p, J=5.9 Hz, 2H), 1.25 (d, J=16.2 Hz, 24H), 0.86 (t, J=6.8 Hz, 6H), 0.68 (t, J=7.5 Hz, 4H), 0.20 (s, 12H). ¹³C NMR (101 MHz, cdcl₃) δ 157.61, 144.34, 134.94, 133.98, 111.99, 87.55, 65.33, 33.50, 31.89, 29.22, 29.07, 23.76, 22.64, 15.70, 14.09, -2.93.

### Example 23: Synthesis of 4-triethylmethylphenyl triflate

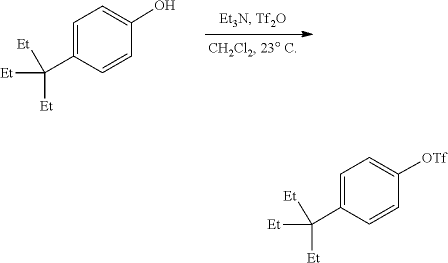 [SMILES: CCC(CC)(CC)c1ccc(O)cc1 ; CCC(CC)(CC)c1ccc(OS(=O)(=O)C(F)(F)F)cc1]

In a continuous purge nitrogen filled glovebox to a solution of the phenol (7.127 g, 37.06 mmol, 1.00 eq) and anhydrous Et₃N (10.0 mL, 111.2 mmol, 3.00 eq) in anhydrous CH₂Cl₂ (120 mL) at 23° C. was added a solution of Tf₂O (9.4 mL, 55.59 mmol, 1.50 eq) in CH₂Cl₂ (30 mL) in a dropwise manner over 20 mins. After stirring (500 rpm) for 12 hours at 23° C., the black mixture was removed from the glovebox, neutralized with an aqueous saturated mixture of NaHCO₃ (100 mL), stirred for 2 mins, poured into a separatory funnel, partitioned, organics washed with aqueous NaHCO₃ (2×50 mL), residual organics were extracted from the aqueous layer using CH₂Cl₂ (2×25 mL), combined, dried over solid Na₂SO₄, decanted, concentrated onto diatomaceous earth, and purified via silica gel chromatography; hexanes −40% CH₂Cl₂ to afford the 4-triethylmethylphenol as a clear colorless oil (9.789 g, 30.18 mmol, 81%). NMR indicated product. ¹H NMR (400 MHz, CDCl₃) δ 7.37-7.32 (m, 2H), 7.21-7.15 (m, 2H), 1.65 (q, J=7.4 Hz, 6H), 0.63 (t, J=7.4 Hz, 9H). ¹⁹F NMR (376 MHz, CDCl₃) δ −72.99. ¹³C NMR (101 MHz, CDCl₃) δ 148.09, 147.23, 128.64, 120.39, 118.73 (d, J=320.8 Hz), 43.83, 28.67, 7.84.

### Example 24: Synthesis of 4-triethylmethylphenyl boropinacolate ester

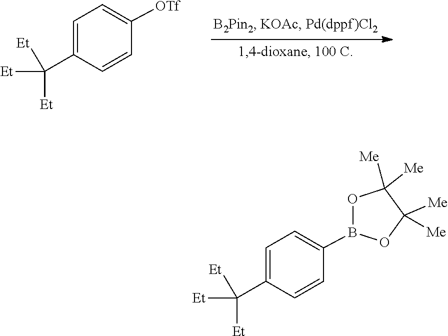 [SMILES: CCC(CC)(CC)c1ccc(OS(=O)(=O)C(F)(F)F)cc1 ; CCC(CC)(CC)c1ccc(B2OC(C)(C)C(C)(C)O2)cc1]

In a continuous purge nitrogen filled glovebox a mixture of the triflate (9.789 g, 30.180 mmol, 1.00 eq), Pd(dppf)Cl₂ (1.232 g, 1.509 mmol, 0.05 eq), B₂Pin₂ (11.496 g, 45.269 mmol, 1.50 eq), and KOAc (8.886 g, 90.540 mmol, 3.00 eq) in anhydrous deoxygenated 1,4-dioxane (100 mL) quipped with a reflux condenser and rubber septa was removed from the glovebox, placed under nitrogen and then placed in a mantle heated to 100° C., stirred vigorously (1000 rpm) for 24 hours, removed from the heating mantle, allowed to cool gradually to 23° C., suction filtered through a pad of silica gel, rinsed with CH₂Cl₂ (4×25 mL), the resulting dark purple/black filtrate solution was concentrated onto diatomaceous earth, and purified via silica gel chromatography; hexanes—60% CH₂Cl₂ in hexanes to afford the boropinacolate ester as a white solid (8.210 g, 27.162 mmol, 90%). NMR indicated product. ¹H NMR (400 MHz, CDCl₃) δ 7.76-7.72 (m, 2H), 7.32-7.28 (m, 2H), 1.67 (q, J=7.4 Hz, 6H), 1.33 (s, 12H), 0.63 (t, J=7.4 Hz, 9H). ¹³C NMR (101 MHz, CDCl₃) δ 150.92, 134.35, 127.69, 126.34, 83.54, 43.95, 28.55, 24.87, 7.93.

### Example 25: Synthesis of 4-(4′-triethylmethylphenyl)phenoxymethyl ethyl ether

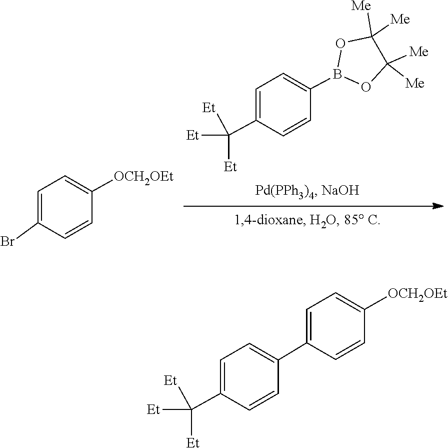

A mixture of boropinacolate ester (2.000 g, 6.617 mmol, 1.50 eq), the aryl bromide (1.019 g, 4.411 mmol, 1.00 eq), NaOH (0.794 g, 19.850 mmol, 4.50 eq), and Pd(PPh₃)₄(0.510 g, 0.4411 mmol, 0.10 eq) in a flask equipped with a reflux condenser was evacuated, back-filled with nitrogen, and the evacuation/refill process was repeated 3x more. Freshly sparged 1,4-dioxane (50 mL) and H₂O (10 mL) were added, the mixture was placed in a mantle heated to 85° C., stirred (500 rpm) for 40 hours, removed from the mantle, allowed to cool to ambient temperature, diluted with CH₂Cl₂ (20 mL), suction filtered through a pad of silica gel, rinsed with CH₂Cl₂ (4×20 mL), the filtrate was concentrated onto diatomaceous earth, and purified via silica gel chromatography using an ISCO; hexanes—50% CH₂Cl₂ in hexanes to afford the biaryl protected phenol as a white solid (1.388 g, 4.251 mmol, 96%). NMR indicated product. ¹H NMR (400 MHz, cdcl₃) δ 7.56-7.51 (m, 2H), 7.51-7.45 (m, 2H), 7.36-7.31 (m, 2H), 7.12-7.05 (m, 2H), 5.25 (s, 2H), 3.75 (q, J=7.1 Hz, 2H), 1.69 (q, J=7.5 Hz, 6H), 1.24 (t, J=7.1 Hz, 3H), 0.68 (t, J=7.4 Hz, 9H). ¹³C NMR (101 MHz, cdcl₃) δ 156.66, 145.94, 137.29, 134.68, 127.87, 127.24, 126.04, 116.43, 93.22, 64.23, 43.50, 28.67, 15.12, 8.01.

### Example 26: Synthesis of 2-iodo-4-(4′-triethylmethylphenyl)phenoxymethyl ethyl ether

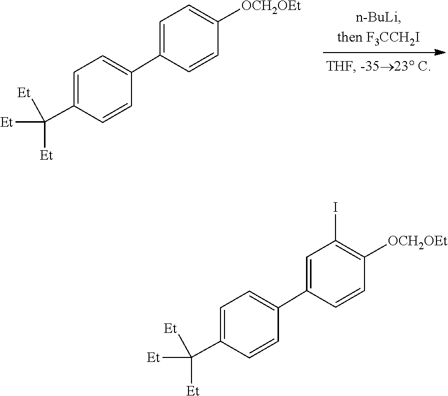

Prior to use, the ether was azeotropically dried using toluene (4×10 mL). A clear, colorless solution of the ether (1.588 g, 4.864 mmol, 1.00 eq) in anhydrous deoxygenated THE (50 mL) in a continuous purge nitrogen filled glovebox was placed in a freezer (−35° C.) for 16 hours. n-BuLi (2.50 mL, 6.323 mmol, 1.30 eq, 2.5 M in hexanes) was added, the amber solution was allowed to sit in the freezer for 10 hours, then 2-iodo-1,1,1-trifluoroethane (0.72 mL, 7.2296 mmol, 1.50 eq) was added neat in a quick dropwise manner, the now golden brown solution was allowed to remain in the freezer for 60 mins, removed, stirred (500 rpm) for 2 hours at 23° C., the mixture was removed from the glovebox, neutralized with H₂O (50 mL), and THE was removed via rotary evaporation. The golden brown mixture was diluted with CH₂Cl₂ (50 mL) and water (50 mL), poured into a separatory funnel, partitioned, organics were extracted with CH₂Cl₂ (2×25 mL), combined, dried over Na₂SO₄, decanted, concentrated onto diatomaceous earth, and purified by silica gel chromatography using an ISCO; 5%-75% CH₂Cl₂ in hexanes to afford the iodide as a clear colorless oil (1.557 g, 3.442 mmol, 71%). NMR indicated product. ¹H NMR (400 MHz, cdcl₃) δ 8.05-7.99 (m, 1H), 7.51 (dt, J=8.5, 1.5 Hz, 1H), 7.47-7.42 (m, 2H), 7.36-7.31 (m, 2H), 7.13 (d, J=8.5 Hz, 1H), 5.31 (d, J=0.9 Hz, 2H), 3.83-3.75 (m, 2H), 1.69 (q, J=7.4 Hz, 6H), 1.24 (td, J=7.1, 0.9 Hz, 3H), 0.71-0.60 (m, 9H). ¹³C NMR (101 MHz, cdcl₃) δ 155.34, 146.57, 137.74, 136.73, 135.89, 127.86, 127.34, 126.10, 114.91, 93.76, 87.54, 64.71, 43.56, 28.66, 15.08, 8.00.

### Example 27: Synthesis of 2-iodo-4-(4′-triethylmethylphenyl)phenol

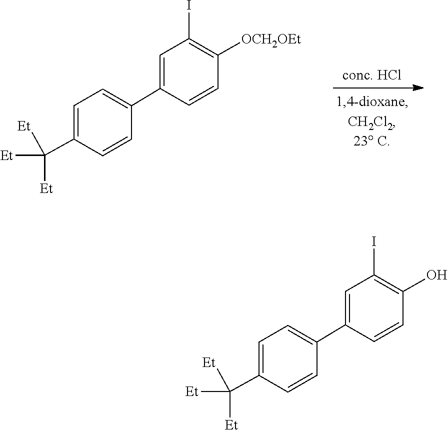

To a clear, colorless solution of the ether (1.557 g, 3.442 mmol, 1.00 eq) in 1,4-dioxane (10 mL) and CH₂Cl₂ (10 mL) under nitrogen at 23° C. was added conc. HCl (10 mL), the mixture was stirred (300 rpm) for 16 hours, water (30 mL) and CH₂Cl₂ (30 mL) was added to the golden brown mixture, which was poured into a separatory funnel, partitioned, organics were extracted with CH₂Cl₂ (2×25 mL), combined, dried over Na₂SO₄, decanted, and concentrated to afford the iodophenol as a clear amber amorphous foam (1.250 g, 3.170 mmol, 92%). NMR indicated product. The crude material was used in the subsequent reaction without further purification. ¹H NMR (400 MHz, cdcl₃) δ 7.89 (d, J=2.1 Hz, 1H), 7.48 (dd, J=8.4, 2.2 Hz, 1H), 7.45-7.40 (m, 2H), 7.35-7.29 (m, 2H), 7.03 (d, J=8.4 Hz, 1H), 5.26 (s, 1H), 1.68 (q, J=7.4 Hz, 6H), 0.66 (t, J=7.4 Hz, 9H).

### Example 28: Synthesis of 1,3-bis[2-iodo-4-(4′-triethylmethylphenyl)phenoxy]propane

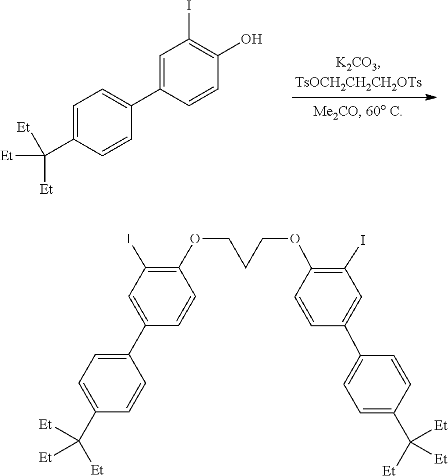 [SMILES: CCC(CC)(CC)c1ccc(-c2ccc(O)c(I)c2)cc1 ; CCC(CC)(CC)c1ccc(-c2ccc(OCCCOc3ccc(-c4ccc(C(CC)(CC)CC)cc4)cc3I)c(I)c2)cc1]

A mixture of the iodophenol (2.271 g, 5.760 mmol, 2.15 eq), K₂CO₃(2.592 g, 18.753 mmol, 7.00 eq), and the bistosylate (1.030 g, 2.679 mmol, 1.00 eq) in acetone (30 mL) was equipped with a reflux condenser, placed under nitrogen, placed in a mantle heated to 60° C., and stirred (500 rpm) for 48 hours. The mixture was then removed from the mantle, allowed to cool to ambient temperature, diluted with CH₂Cl₂ (20 mL), the mixture was suction filtered through a pad of diatomaceous earth, rinsed with CH₂Cl₂ (4×20 mL), the pale golden brown filtrate solution was poured into a separatory funnel, washed with aqueous NaOH (2×20 mL, 1 N), residual organics were extracted from the aqueous using CH₂Cl₂ (2×20 mL), dried over Na₂SO₄, decanted, the pale golden brown solution was concentrated, CH₂Cl₂ (10 mL) was added, the solution was suction filtered through a pad of silica gel, rinsed with CH₂Cl₂ (4×20 mL), and the filtrate was concentrated to afford the bisiodide as a white solid (2.150 g, 2.595 mmol, 97%). NMR indicated product. ¹H NMR (400 MHz, cdcl₃) δ 8.01 (d, J=2.2 Hz, 2H), 7.52 (dd, J=8.5, 2.2 Hz, 2H), 7.48-7.41 (m, 4H), 7.38-7.27 (m, 4H), 6.93 (d, J=8.5 Hz, 2H), 4.34 (t, J=5.9 Hz, 4H), 2.39 (p, J=5.9 Hz, 2H), 1.69 (q, J=7.4 Hz, 12H), 0.67 (t, J=7.4 Hz, 18H). ¹³C NMR (101 MHz, cdcl₃) δ 156.50, 146.44, 137.71, 135.94, 135.81, 127.88, 127.33, 126.05, 112.28, 87.08, 65.73, 43.55, 29.21, 28.66, 8.00.

### Example 29: Synthesis of Compound 1: a compound of formula (I) wherein R¹, R², and R⁵ to R⁹ are H, R³ and R⁴ are each (CH₃)₃CCH₂C(CH₃)₂— (“t-Octyl”),and each R¹⁰ is tertiary-butyl

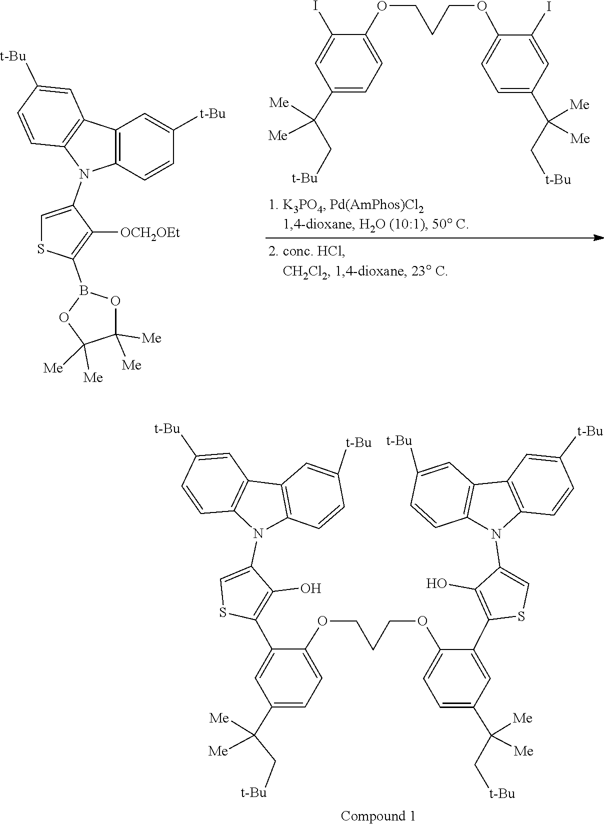

A solid mixture of the boropinacolate ester (0.872 g, 1.086 mmol, 3.00 eq, ~70% pure), bis-iodide (0.255 g, 0.3621 mmol, 1.00 eq), Pd(AmPhos)Cl₂ (52.0 mg, 0.0724 mmol, 0.20 eq), and solid K₃PO₄ (0.692 g, 3.259 mmol, 9.00 eq) in a round-bottom flask equipped with a reflux condenser sealed with a rubber septa was evacuated, back-filled with nitrogen, the evacuation/nitrogen re-fill process was repeated 3× more, then freshly sparged deoxygenated 1,4-dioxane (11 mL) and H₂O (1 mL) were added via syringe, and the resultant canary yellow mixture was placed in a mantle heated to 50° C. After stirring (300 rpm) for 36 hours, the now black mixture was removed from the mantle, allowed to cool to 23° C., diluted with CH₂Cl₂ (20 mL), the biphasic mixture was suction filtered through a pad of silica gel, rinsed with CH₂Cl₂ (4×20 mL), the filtrate was concentrated onto diatomaceous earth, and purified via silica gel chromatography; 10%-50% CH₂Cl₂ in hexanes to afford the protected coupled product as a pale golden brown amorphous foam (0.450 g). NMR indicated product with minor impurities. The mixture was used in the subsequent reaction without further purification. To a solution of the aforementioned coupled product (0.450 g) in 1,4-dioxane and CH₂Cl₂ (10 mL, 1:1) under nitrogen at 23° C. was added aqueous conc. HCl (5 mL, 37% w/w). After stirring (300 rpm) for 16 hours, the dark brown mixture was diluted with water (25 mL) and CH₂Cl₂ (25 mL), the biphasic mixture was poured into a separatory funnel, partitioned, residual organics were extracted with CH₂Cl₂ (2×25 mL), combined, dried over solid Na₂SO₄, decanted, concentrated onto diatomaceous earth, and purified via silica gel chromatography; 10%-50% CH₂Cl₂ in hexanes to afford the hydroxythiophene as a pale yellow solid (0.305 g, 0.2537 mmol, 70% two steps). NMR indicated product. ¹H NMR (500 MHz, cdcl₃) δ 8.13 (dd, J=2.0, 0.6 Hz, 4H), 7.57 (d, J=2.4 Hz, 2H), 7.43 (dd, J=8.6, 1.9 Hz, 4H), 7.33 (s, 2H), 7.26-7.24 (m, 4H), 7.23 (d, J=0.6 Hz, 2H), 7.17 (dd, J=8.7, 2.4 Hz, 2H), 6.75 (d, J=8.7 Hz, 2H), 4.23 (t, J=5.8 Hz, 4H), 2.22 (p, J=5.8 Hz, 2H), 1.75 (s, 4H), 1.46 (s, 36H), 1.38 (s, 12H), 0.75 (s, 18H). ¹³C NMR (126 MHz, cdcl₃) δ 151.47, 146.16, 144.97, 142.66, 139.68, 128.28, 127.55, 126.67, 123.46, 123.17, 121.99, 119.80, 116.18, 115.81, 113.92, 109.61, 66.78, 56.86, 38.20, 34.72, 32.39, 32.05, 31.89, 31.59, 29.01.

### Example 30: Synthesis of Compound 2: a compound of formula (I) wherein R¹, R², and R⁵ to R⁹ are H, R³ and R⁴ are each octyloxy, and each R¹⁰ is tertiary-butyl

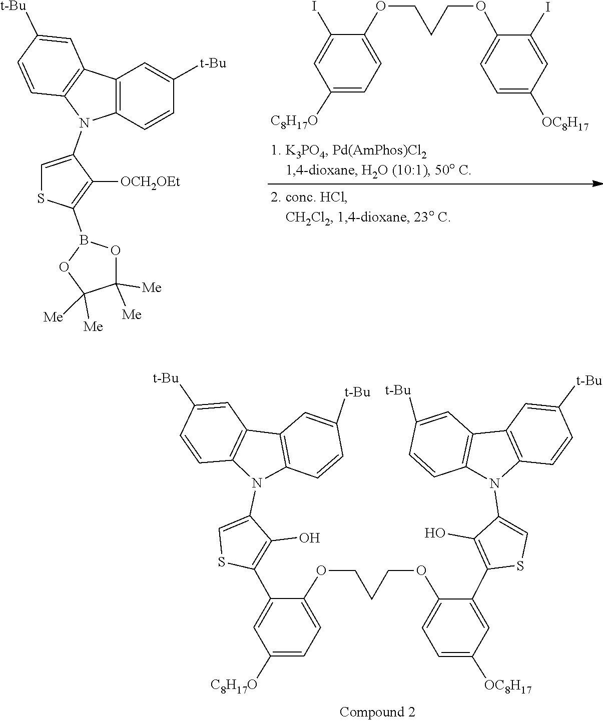

A solid mixture of the boropinacolate ester (17.973 g, 22.403 mmol, 3.00 eq, ~70% pure), bis-iodide (5.500 g, 7.468 mmol, 1.00 eq), Pd(AmPhos)Cl₂ (1.058 g, 1.494 mmol, 0.20 eq), and solid K₃PO₄ (14.267 g, 67.212 mmol, 9.00 eq) in a round-bottom flask equipped with a reflux condenser sealed with a rubber septa was evacuated, back-filled with nitrogen, the evacuation/nitrogen re-fill process was repeated 3× more, then freshly sparged deoxygenated 1,4-dioxane (70 mL) and H₂O (7 mL) were added via syringe, and the resultant canary yellow mixture was placed in a mantle heated to 50° C. After stirring (300 rpm) for 36 hours, the now black mixture was removed from the mantle, allowed to cool to 23° C., silica gel (50 g) was added, the mixture was diluted with CH₂Cl₂ (50 mL), the biphasic mixture was suction filtered through a pad of diatomaceous earth, rinsed with CH₂Cl₂ (4×30 mL), the filtrate was concentrated onto diatomaceous earth, and purified via silica gel chromatography; 15%-55% CH₂Cl₂ in hexanes to afford the protected coupled product as a dark red amorphous foam (6.805 g). NMR indicated product with minor impurities. The mixture was used in the subsequent reaction without further purification. To a solution of the aforementioned coupled product (5.805 g) in 1,4-dioxane and CH₂Cl₂ 60 mL, 1:1) under nitrogen at 23° C. was added aqueous conc. HCl (20 mL, 37% w/w). After stirring (300 rpm) for 20 hours, the dark red-black mixture was diluted with water (50 mL) and CH₂Cl₂ (50 mL), the biphasic mixture was poured into a separatory funnel, partitioned, residual organics were extracted with CH₂Cl₂ (2×25 mL), combined, dried over solid Na₂SO₄, decanted, concentrated onto diatomaceous earth, and purified via silica gel chromatography; 20%-35% CH₂Cl₂ in hexanes to afford the hydroxythiophene as a off-white foam (5.814 g, 4.705 mmol, 63% two steps). NMR indicated product. ¹H NMR (500 MHz, cdcl₃) δ 8.12 (dd, J=2.0, 0.6 Hz, 4H), 7.58 (s, 2H), 7.41 (dd, J=8.6, 1.9 Hz, 4H), 7.34 (s, 2H), 7.19 (dd, J=8.7, 0.6 Hz, 4H), 7.12 (d, J=2.9 Hz, 2H), 6.78 (d, J=9.0 Hz, 2H), 6.70 (dd, J=9.0, 3.0 Hz, 2H), 4.15 (t, J=5.9 Hz, 4H), 3.96 (t, J=6.5 Hz, 4H), 2.14 (p, J=5.8 Hz, 2H), 1.80 (p, J=6.7 Hz, 4H), 1.46 (s, 36H), 1.53-1.26 (m, 20H), 0.90 (td, J=6.4, 4.7 Hz, 6H). ¹³C NMR (126 MHz, cdcl₃) δ 155.08, 147.62, 146.57, 142.68, 139.62, 127.70, 124.49, 123.49, 123.17, 120.18, 117.13, 116.17, 115.84, 115.09, 114.66, 109.58, 68.62, 68.27, 34.71, 32.04, 31.84, 29.39, 29.33, 29.27, 28.94, 26.08, 22.68, 14.12.

### Example 31: Synthesis of Compound 3: a compound of formula (I) wherein R¹, R², and R⁵ to R⁹ are H, R³ and R⁴ are each triethylmethyl, and each R¹⁰ is tertiary-butyl

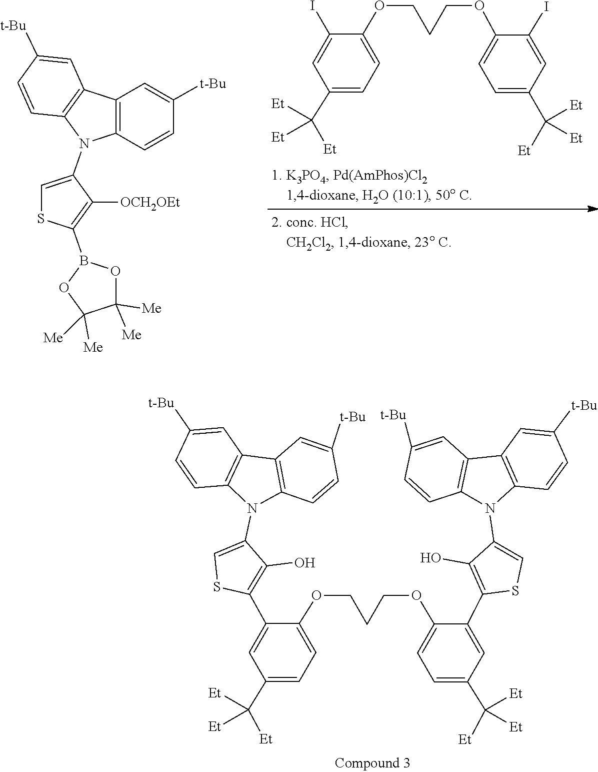

A solid mixture of the boropinacolate ester (1.200 g, 0.9443 mmol, 3.00 eq, ~65% pure), bis-iodide (0.213 g, 0.3148 mmol, 1.00 eq), Pd(AmPhos)Cl₂ (44.6 mg, 0.0630 mmol, 0.20 eq), and solid K₃PO₄ (0.601 g, 2.833 mmol, 9.00 eq) in a round-bottom flask equipped with a reflux condenser sealed with a rubber septa was evacuated, back-filled with nitrogen, the evacuation/nitrogen re-fill process was repeated 3× more, then freshly sparged deoxygenated 1,4-dioxane (15 mL) and H₂O (1.5 mL) were added via syringe, and the resultant canary yellow mixture was placed in a mantle heated to 50° C. After stirring (300 rpm) for 36 hours, the now black mixture was removed from the mantle, allowed to cool to 23° C., diluted with CH₂Cl₂ (20 mL), the biphasic mixture was suction filtered through a pad of silica gel, rinsed with CH₂Cl₂ (4×20 mL), the filtrate was concentrated onto diatomaceous earth, and purified via silica gel chromatography; 10%-50% CH₂Cl₂ in hexanes to afford the protected coupled product as a dark brown amorphous foam (0.471 g). NMR indicated product with minor impurities. The mixture was used in the subsequent reaction without further purification. To a solution of the aforementioned coupled product (0.471 g) in 1,4-dioxane and CH₂Cl₂ (10 mL, 1:1) under nitrogen at 23° C. was added aqueous conc. HCl (5 mL, 37% w/w). After stirring (300 rpm) for 20 hours, the dark brown mixture was diluted with water (25 mL) and CH₂Cl₂ (25 mL), the biphasic mixture was poured into a separatory funnel, partitioned, residual organics were extracted with CH₂Cl₂ (2×25 mL), combined, dried over solid Na₂SO₄, decanted, concentrated onto diatomaceous earth, and purified via silica gel chromatography; 10%-50% CH₂Cl₂ in hexanes to afford the hydroxythiophene as an off-white solid (0.299 g, 0.2543 mmol, 81% two steps). NMR indicated product. ¹H NMR (400 MHz, cdcl₃) δ 8.12 (d, J=1.9 Hz, 4H), 7.49 (d, J=2.3 Hz, 2H), 7.43 (dd, J=8.6, 1.9 Hz, 4H), 7.32 (s, 2H), 7.23 (d, J=8.7 Hz, 4H), 7.10 (dd, J=8.7, 2.4 Hz, 2H), 6.77 (d, J=8.7 Hz, 2H), 4.24 (t, J=5.8 Hz, 4H), 2.23 (t, J=5.9 Hz, 2H), 1.67 (q, J=7.3 Hz, 12H), 1.45 (s, 36H), 0.67 (t, J=7.3 Hz, 18H). ¹³C NMR (101 MHz, cdcl₃) δ 151.36, 146.14, 142.65, 142.15, 139.69, 129.01, 127.54, 127.44, 123.45, 123.17, 122.09, 119.73, 116.16, 115.93, 114.01, 109.62, 66.74, 43.36, 34.70, 32.04, 29.03, 28.74, 8.02.

### Example 32: Synthesis of Compound 4: A Compound of Formula (I) Wherein R¹, R², and R⁵ to R⁹ are H, R³ and R⁴ are each 3,5-di-tert-butylphenyl (“3,5-di(t-Bu)Ph”), and each R¹⁰ is tertiary-butyl

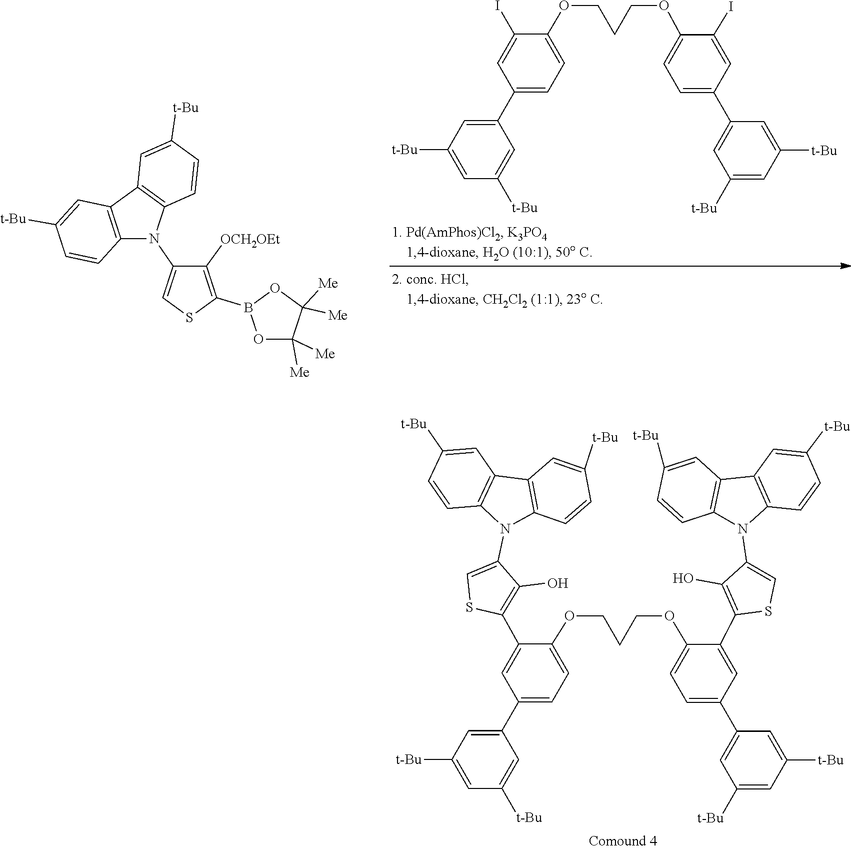

A solid mixture of the boropinacolate ester (1.278 g, 1.366 mmol, 3.00 eq), bis-iodide (0.390 g, 0.4552 mmol, 1.00 eq), Pd(AmPhos)Cl₂ (65.0 mg, 0.09104 mmol, 0.20 eq), and solid K₃PO₄ (0.870 g, 4.097 mmol, 9.00 eq) in a round-bottom flask equipped with a reflux condenser sealed with a rubber septa was evacuated, back-filled with nitrogen, the evacuation/nitrogen re-fill process was repeated 3× more, then freshly sparged deoxygenated 1,4-dioxane (20 mL) and H₂O (2.0 mL) were added via syringe, and the resultant canary yellow mixture was placed in a mantle heated to 50° C. After stirring (300 rpm) for 36 hours, the now black mixture was removed from the mantle, allowed to cool to 23° C., diluted with CH₂Cl₂ (20 mL), the biphasic mixture was suction filtered through a pad of silica gel, rinsed with CH₂Cl₂ (4×20 mL), the filtrate was concentrated onto diatomaceous earth, and purified via silica gel chromatography; 10%-60% CH₂Cl₂ in hexanes to afford the protected coupled product as a dark red amorphous foam (0.697 g). NMR indicated product with minor impurities. The mixture was used in the subsequent reaction without further purification. To a solution of the aforementioned coupled product (0.697 g) in 1,4-dioxane and CH₂Cl₂(14 mL, 1:1) under nitrogen at 23° C. was added aqueous conc. HCl (7 mL, 37% w/w). After stirring (300 rpm) for 20 hours, the dark red-black mixture was diluted with water (25 mL) and CH₂Cl₂ (25 mL), the biphasic mixture was poured into a separatory funnel, partitioned, residual organics were extracted with CH₂Cl₂ (2×25 mL), combined, dried over solid Na₂SO₄, decanted, concentrated onto diatomaceous earth, and purified via silica gel chromatography; 10%-60% CH₂Cl₂ in hexanes to afford the hydroxythiophene as a grey off-white amorphous foam (0.475 g, 0.3503 mmol, 77% two steps). NMR indicated product. ¹H NMR (500 MHz, cdcl₃) δ 8.16 (d, J=1.7 Hz, 4H), 7.84 (d, J=2.3 Hz, 2H), 7.50-7.46 (m, 4H), 7.46-7.43 (m, 8H), 7.40 (s, 2H), 7.32-7.27 (m, 4H), 7.21 (s, 2H), 6.97 (d, J=8.5 Hz, 2H), 4.36 (t, J=5.8 Hz, 4H), 2.34 (p, J=5.8 Hz, 2H), 1.47 (s, 36H), 1.43 (s, 36H). ¹³C NMR (126 MHz, cdcl₃) δ 153.05, 151.28, 146.49, 142.78, 139.66, 137.73, 129.57, 127.93, 127.54, 123.54, 123.24, 123.20, 121.59, 121.52, 120.20, 116.24, 115.26, 114.81, 109.63, 67.09, 35.03, 34.74, 32.07, 31.59, 29.02.

### Example 33: Synthesis of Compound 5: a compound of formula (I) wherein R¹, R², and R⁵ to R⁹ are H, R³ and R⁴ are each CH₃(CH₂)₇,(CH₃)₂Si— (octyl-dimethyl-silyl), and each R¹⁰ is tertiary-butyl

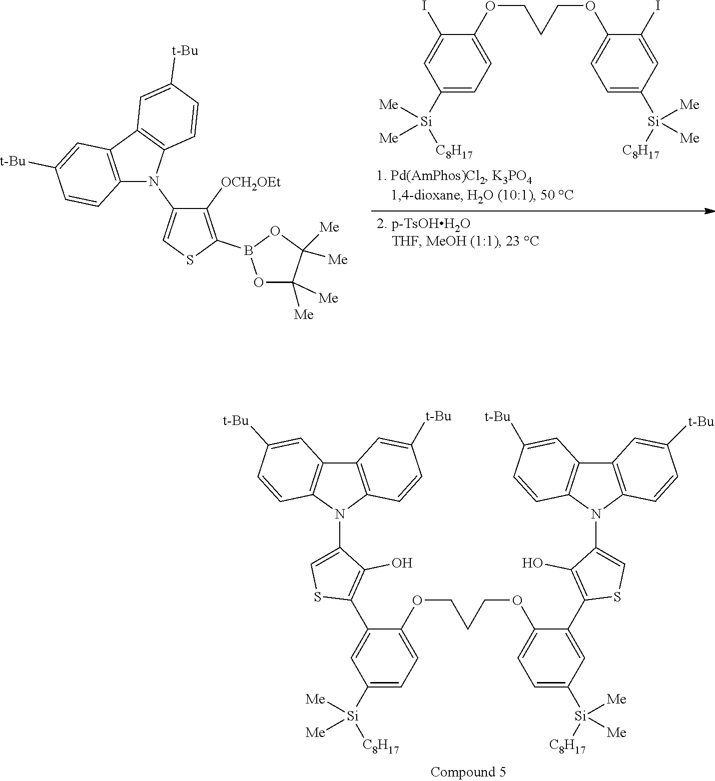

A solid mixture of the boropinacolate ester (1.230 g, 1.314 mmol, 3.00 eq, approx. 60% pure), bis-iodide (0.360 g, 0.4380 mmol, 1.00 eq), Pd(AmPhos)Cl₂ (69.0 mg, 0.09734 mmol, 0.20 eq), and solid K₃PO₄ (0.930 g, 4.380 mmol, 9.00 eq) in a round-bottom flask equipped with a reflux condenser sealed with a rubber septa was evacuated, back-filled with nitrogen, the evacuation/nitrogen re-fill process was repeated 3× more, then freshly sparged deoxygenated 1,4-dioxane (15 mL) and H₂O (1.5 mL) were added via syringe, and the resultant canary yellow mixture was placed in a mantle heated to 50° C. After stirring (300 rpm) for 36 hours, the now black mixture was removed from the mantle, allowed to cool to 23° C., diluted with CH₂Cl₂ (20 mL), the biphasic mixture was suction filtered through a pad of silica gel, rinsed with CH₂Cl₂ (4×20 mL), the filtrate was concentrated onto diatomaceous earth, and purified via silica gel chromatography; 10%-35% CH₂Cl₂ in hexanes to afford the protected coupled product as a clear pale yellow oil (0.561 g). NMR indicated product with minor impurities. The mixture was used in the subsequent reaction without further purification. To a solution of the aforementioned coupled product (0.561 g) in THF and MeOH (14 mL, 1:1) under nitrogen at 23° C. was added aqueous para-toluene sulfonic acid monohydrate (0.333 g, 1.752 mmol, 4.00 eq). After stirring (300 rpm) for 96 hours, the golden yellow solution was diluted with an aqueous phosphate buffer (25 mL, pH=7) and CH₂Cl₂ (25 mL), the biphasic mixture was poured into a separatory funnel, partitioned, residual organics were extracted with CH₂Cl₂ (2×25 mL), combined, dried over solid Na₂SO₄, decanted, concentrated onto diatomaceous earth, and purified via silica gel chromatography; 10%-35% CH₂Cl₂ in hexanes to afford the hydroxythiophene as a white amorphous foam (0.224 g, 0.1697 mmol, 39% two steps). NMR indicated product. ¹H NMR (400 MHz, cdcl₃) δ 8.12 (d, J=1.9 Hz, 4H), 7.69 (d, J=1.6 Hz, 2H), 7.41 (dd, J=8.6, 1.9 Hz, 4H), 7.33 (s, 2H), 7.31 (dd, J=8.2, 1.6 Hz, 2H), 7.22 (d, J=8.6 Hz, 4H), 7.00 (s, 2H), 6.82 (d, J=8.2 Hz, 2H), 4.25 (t, J=5.8 Hz, 4H), 2.29-2.20 (m, 2H), 1.45 (s, 36H), 1.37-1.20 (m, 24H), 0.87 (d, J=6.6 Hz, 6H), 0.75 (t, J=7.8 Hz, 4H), 0.27 (s, 12H).

### Example 34: Synthesis of Compound 6: a compound of formula (I) wherein R¹, R² and R⁵ to R⁹ are H, R³ and R⁴ are each 4-triethylmethylphenyl (“4-(Et₃C)Ph” or “Ph-4-(CEt₃)”), and each R¹⁰ is tertiary-butyl

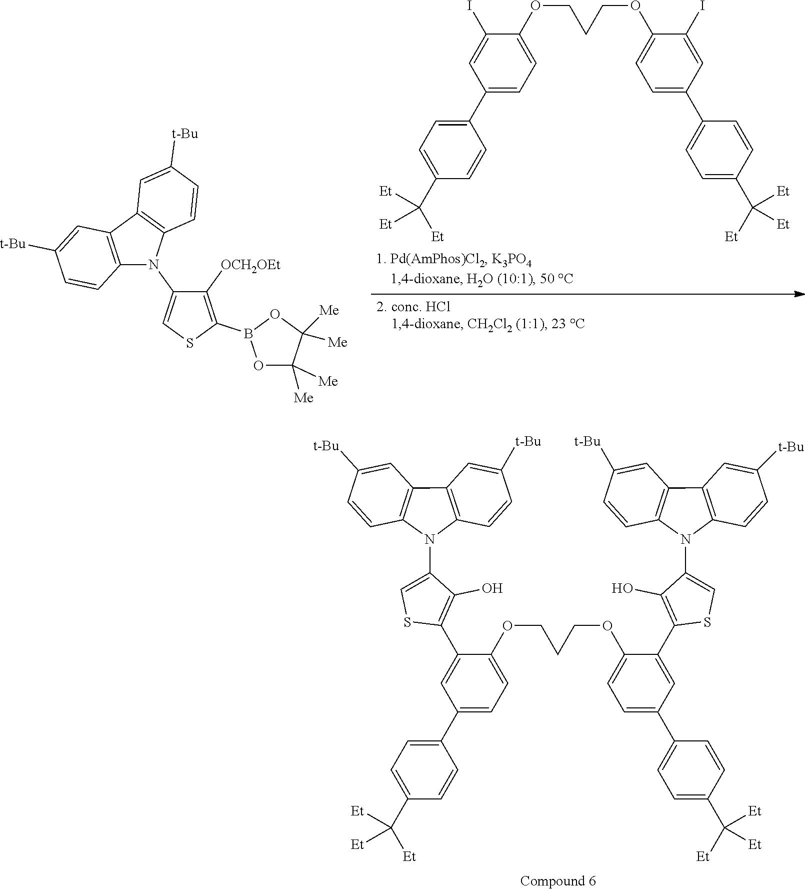

A solid mixture of the boropinacolate ester (1.017 g, 1.086 mmol, 3.00 eq, approx. 60% pure), bis-iodide (0.300 g, 0.3620 mmol, 1.00 eq), Pd(AmPhos)Cl₂ (51.3 mg, 0.07240 mmol, 0.20 eq), and solid K₃PO₄ (0.768 g, 3.620 mmol, 9.00 eq) in a round-bottom flask equipped with a reflux condenser sealed with a rubber septa was evacuated, back-filled with nitrogen, the evacuation/nitrogen re-fill process was repeated 3× more, then freshly sparged deoxygenated 1,4-dioxane (15 mL) and H₂O (1.5 mL) were added via syringe, and the resultant canary yellow mixture was placed in a mantle heated to 50° C. After stirring (300 rpm) for 36 hours, the now black mixture was removed from the mantle, allowed to cool to 23° C., diluted with CH₂Cl₂ (20 mL), the biphasic mixture was suction filtered through a pad of silica gel, rinsed with CH₂Cl₂ (4×20 mL), the filtrate was concentrated onto diatomaceous earth, and purified via silica gel chromatography; 10%-60% CH₂Cl₂ in hexanes to afford the protected coupled product as a golden brown amorphous foam (0.403 g). NMR indicated product with minor impurities. The mixture was used in the subsequent reaction without further purification. To a solution of the aforementioned coupled product (0.403 g) in 1,4-dioxane and CH₂Cl₂ (10 mL, 1:1) under nitrogen at 23° C. was added aqueous conc. HCl (5 mL, 37% w/w). After stirring (300 rpm) for 20 hours, the dark red-black mixture was diluted with water (25 mL) and CH₂Cl₂ (25 mL), the biphasic mixture was poured into a separatory funnel, partitioned, residual organics were extracted with CH₂Cl₂ (2×25 mL), combined, dried over solid Na₂SO₄, decanted, concentrated onto diatomaceous earth, and purified via silica gel chromatography; 10%-60% CH₂Cl₂ in hexanes to afford the hydroxythiophene as an off-white solid (0.294 g, 0.2214 mmol, 61% two steps). NMR indicated product. ¹H NMR (500 MHz, cdcl₃) δ 8.12 (dd, J=2.0, 0.7 Hz, 4H), 7.82 (d, J=2.3 Hz, 2H), 7.53-7.49 (m, 4H), 7.42 (dd, J=8.6, 1.9 Hz, 4H), 7.39-7.36 (m, 8H), 7.25 (d, J=0.6 Hz, 2H), 7.24 (d, J=0.6 Hz, 2H), 7.18 (s, 2H), 6.90 (d, J=8.6 Hz, 2H), 4.30 (t, J=5.9 Hz, 4H), 2.28 (p, J=5.9 Hz, 2H), 1.72 (q, J=7.5 Hz, 12H), 1.44 (s, 36H), 0.70 (t, J=7.4 Hz, 18H). ¹³C NMR (126 MHz, cdcl₃) δ 152.89, 146.59, 146.39, 142.73, 139.60, 136.63, 136.17, 129.00, 127.47, 127.37, 127.30, 126.29, 123.53, 123.18, 120.11, 116.21, 115.23, 114.82, 109.58, 66.95, 43.60, 34.71, 32.04, 31.60, 28.69, 8.05.

Examples 35 and 36: synthesis of Precatalyst 1: a precatalyst of formula (II) wherein R¹, R², and R⁵ to R⁹ are H, R³ and R⁴ are each (CH₃)₃CCH₂C(CH₃)₂— (“t-Octyl”), each R¹⁰ is tertiary-butyl, M is Zr, each X is benzyl, and subscript n is 2; and Precatalyst 2: a precatalyst of formula (II) wherein R¹, R², and R⁵ to R⁹ are H, R³ and R⁴ are each (CH₃)₃CCH₂C(CH₃)₂— (“t-Octyl”), each R¹⁰ is tertiary-butyl, M is Hf, each X is benzyl, and subscript n is 2.

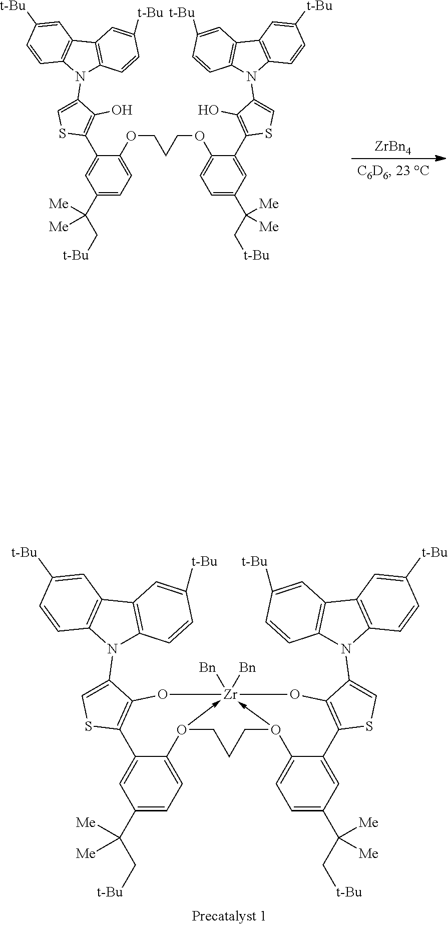

The thiophene ligand was azeotropically dried using PhMe (4×10 mL) prior to use. To a clear colorless solution of the thiophene (10.9 mg, 0.00906 mmol, 1.00 eq) in C₆D₆(1.75 mL) in a continuous purge, nitrogen filled glovebox at 23° C. was added a solution of ZrBn₄ (5.0 mg, 0.0109 mmol, 1.20 eq) in C₆D6 (0.40 mL) in a dropwise manner. After stirring (500 rpm) for 20 mins the golden yellow solution was filtered using a 0.45 μm PTFE submicron filter connected to a 0.20 μm PTFE submicron filter to afford the zirconium complex as a golden yellow solution in C₆D₆. NMR indicated product. ¹H NMR (500 MHz, c₆d₆) δ 8.55-8.52 (m, 2H), 8.15 (dd, J=1.9, 0.6 Hz, 2H), 7.58 (d, J=2.5 Hz, 2H), 7.54 (dd, J=8.5, 1.9 Hz, 2H), 7.46 (dd, J=8.7, 1.9 Hz, 2H), 7.37-7.34 (m, 2H), 7.28 (dd, J=8.7, 0.6 Hz, 2H), 7.08-7.04 (m, 2H), 6.99-6.94 (m, 4H), 6.89 (s, 2H), 6.85-6.80 (m, 2H), 6.28-6.23 (m, 4H), 5.12 (d, J=8.6 Hz, 2H), 3.96 (dt, J=11.2, 6.7 Hz, 2H), 3.09 (dt, J=10.8, 5.2 Hz, 2H), 1.71 (d, J=14.7 Hz, 2H), 1.60 (s, 18H), 1.55 (d, J=14.6 Hz, 2H), 1.25 (s, 18H), 1.21 (s, 12H), 0.99 (d, J=12.2 Hz, 2H), 0.89-0.80 (m, 2H), 0.70 (s, 18H), 0.51 (d, J=12.2 Hz, 2H). ¹³C NMR (126 MHz, c₆d₆) δ 154.38, 152.41, 149.02, 146.56, 142.85, 142.49, 139.53, 139.41, 128.44, 128.33, 126.86, 126.36, 125.10, 124.57, 122.88, 122.35, 122.15, 120.72, 117.31, 116.84, 116.25, 115.67, 112.37, 108.80, 76.49, 74.50, 56.54, 38.30, 34.68, 34.36, 32.13, 31.99, 31.69, 31.67, 30.10, 27.20.

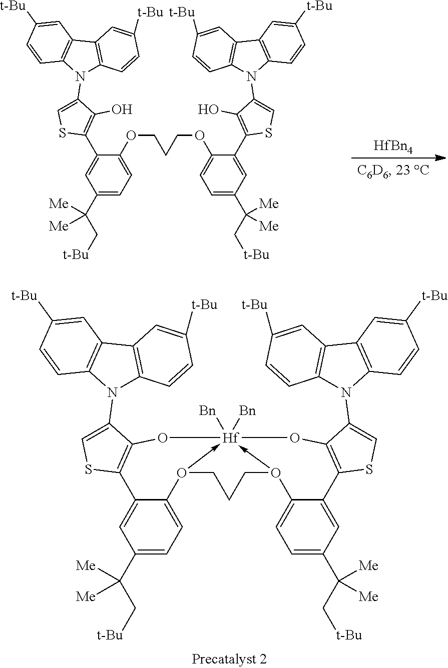

The thiophene ligand was azeotropically dried using PhMe (4×10 mL) prior to use. To a clear colorless solution of the thiophene (10.0 mg, 0.00831 mmol, 1.00 eq) in C₆D₆ (2.90 mL) in a continuous purge, nitrogen filled glovebox at 23° C. was added a solution of HfBn₄ (5.4 mg, 0.00997 mmol, 1.20 eq) in C₆D₆(0.43 mL) in a dropwise manner. After stirring (500 rpm) for 20 mins the pale yellow solution was filtered using a 0.45 μm PTFE submicron filter connected to a 0.20 μm PTFE submicron filter to afford the hafnium complex as a pale yellow solution in C₆D₆. NMR indicated product. ¹H NMR (500 MHz, c₆d₆) δ 8.55 (dd, J=2.0, 0.6 Hz, 2H), 8.16 (dd, J=1.9, 0.7 Hz, 2H), 7.58 (d, J=2.4 Hz, 2H), 7.54 (dd, J=8.5, 1.9 Hz, 2H), 7.45 (dd, J=8.7, 1.9 Hz, 2H), 7.33 (dd, J=8.5, 0.6 Hz, 2H), 7.19 (dd, J=8.7, 0.6 Hz, 2H), 7.11-7.07 (m, 6H), 6.88 (s, 2H), 6.80 (tt, J=7.3, 1.2 Hz, 2H), 6.50 (d, J=7.6 Hz, 2H), 6.28-6.24 (m, 4H), 5.12 (d, J=8.6 Hz, 2H), 3.97 (dt, J=11.3, 6.7 Hz, 2H), 3.09 (dt, J=11.3, 5.3 Hz, 2H), 1.72 (d, J=14.6 Hz, 2H), 1.61 (s, 18H), 1.55 (d, J=14.6 Hz, 2H), 1.25 (s, 18H), 1.22 (s, 6H), 1.21 (s, 6H), 0.85 (dd, J=12.6, 5.6 Hz, 4H), 0.71 (s, 18H), 0.21 (d, J=13.2 Hz, 2H). ¹³C NMR (126 MHz, c₆d₆) δ 153.91, 152.48, 149.37, 147.43, 142.91, 142.49, 139.49, 139.41, 129.88, 128.82, 128.57, 128.37, 127.05, 126.51, 125.19, 124.62, 124.34, 122.84, 122.37, 122.28, 120.71, 117.38, 116.22, 115.60, 112.43, 108.76, 83.01, 76.70, 56.53, 38.34, 34.69, 34.36, 32.15, 32.13, 31.97, 31.71, 31.67, 30.08, 26.94.

Examples 37 and 38: synthesis of Precatalyst 3: a precatalyst of formula (II) wherein R¹, R², and R⁵ to R⁹ are H, R³ and R⁴ are each CH₃(CH₂)₇O— (octyloxy), each R¹⁰ is tertiary-butyl, M is Zr, each X is benzyl, and subscript n is 2; and Precatalyst 4: a precatalyst of formula (II) wherein R¹, R², and R⁵ to R⁹ are H, R³ and R⁴ are each CH₃(CH₂)₇O-(octyloxy), each R¹⁰ is tertiary-butyl, M is Hf, each X is benzyl, and subscript n is 2.

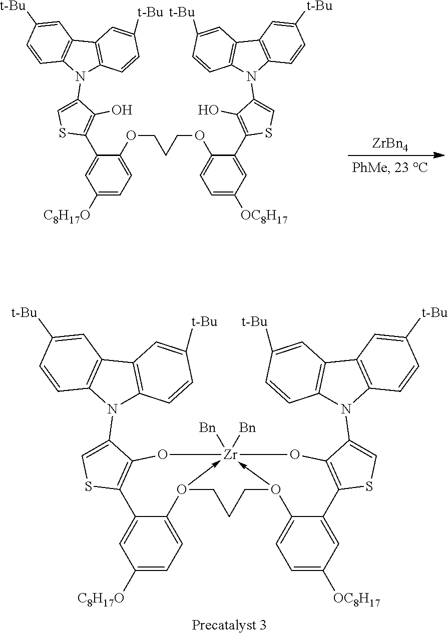

The thiophene ligand was azeotropically dried using PhMe (4×10 mL) prior to use. To a clear colorless solution of the thiophene (153.0 mg, 0.1238 mmol, 1.00 eq) in PhMe (35.5 mL) in a continuous purge, nitrogen filled glovebox at 23° C. was added a solution of ZrBn₄ (62.1 mg, 0.1362 mmol, 1.10 eq) in PhMe (5.0 mL) in a dropwise manner. After stirring (500 rpm) for 15 mins the golden yellow solution was filtered using a 0.45 μm PTFE submicron filter connected to a 0.20 μm PTFE submicron filter, rinsed with PhMe (3×3 mL), and concentrated to afford the zirconium complex as a pale golden brown foam (185.3 mg, 0.1231, 99%). NMR indicated product. ¹H NMR (500 MHz, c₆d₆) δ 8.50 (d, J=1.9 Hz, 2H), 8.27 (d, J=1.9 Hz, 2H), 7.53 (ddd, J=11.4, 8.8, 1.9 Hz, 4H), 7.41 (d, J=8.7 Hz, 2H), 7.33 (d, J=8.4 Hz, 2H), 7.06 (t, J=7.6 Hz, 4H), 7.01 (d, J=3.0 Hz, 2H), 6.88 (s, 2H), 6.78 (t, J=7.3 Hz, 2H), 6.49 (dd, J=8.9, 3.0 Hz, 2H), 6.35 (d, J=7.7 Hz, 4H), 5.01 (d, J=8.9 Hz, 2H), 3.80 (dt, J=11.6, 6.1 Hz, 2H), 3.67-3.59 (m, 2H), 3.55-3.47 (m, 2H), 2.98 (dt, J=10.9, 5.5 Hz, 2H), 1.58-1.48 (m, 4H), 1.52 (s, 18H), 1.32-1.12 (m, 20H), 1.28 (s, 18H), 1.08 (d, J=12.3 Hz, 2H), 0.90 (t, J=7.1 Hz, 8H), 0.66 (d, J=12.2 Hz, 2H). ¹³C NMR (126 MHz, c₆d₆) δ 157.41, 152.57, 149.80, 147.04, 143.07, 142.56, 139.57, 139.52, 128.94, 128.33, 128.17, 126.66, 125.30, 125.07, 124.53, 124.07, 122.90, 122.43, 120.71, 117.59, 116.28, 115.71, 115.49, 112.43, 108.78, 77.28, 74.65, 68.13, 34.61, 34.43, 32.06, 31.84, 31.72, 29.28, 29.27, 29.09, 27.47, 25.91, 22.72, 14.02.

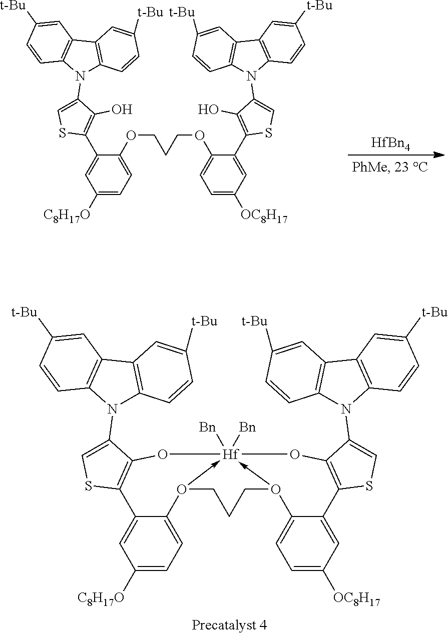

The thiophene ligand was azeotropically dried using PhMe (4×10 mL) prior to use. To a clear colorless solution of the thiophene (50.0 mg, 0.04046 mmol, 1.00 eq) in PhMe (18.3 mL) in a continuous purge, nitrogen filled glovebox at 23° C. was added a solution of HfBn₄ (24.2 mg, 0.04451 mmol, 1.10 eq) in PhMe (1.94 mL) in a dropwise manner. After stirring (500 rpm) for 15 mins the pale yellow solution was filtered using a 0.45 μm PTFE submicron filter connected to a 0.20 μm PTFE submicron filter, rinsed with PhMe (3×3 mL), and concentrated to afford the hafnium complex as a pale golden yellow foam (70.5 mg, 0.04424 mmol, 99%). NMR indicated product. ¹H NMR (500 MHz, c₆d₆) δ 8.52 (dd, J=2.0, 0.6 Hz, 2H), 8.28 (dd, J=1.9, 0.6 Hz, 2H), 7.52 (ddd, J=8.5, 6.7, 1.9 Hz, 4H), 7.32 (td, J=8.6, 0.7 Hz, 4H), 7.11-7.08 (m, 4H), 7.01 (d, J=3.1 Hz, 2H), 6.88 (s, 2H), 6.78-6.74 (m, 2H), 6.52 (dd, J=8.9, 3.1 Hz, 2H), 6.38-6.35 (m, 4H), 5.01 (d, J=8.9 Hz, 2H), 3.80 (dt, J=11.1, 6.2 Hz, 2H), 3.63 (dt, J=9.0, 6.5 Hz, 2H), 3.52 (dt, J=9.0, 6.5 Hz, 2H), 2.98 (dt, J=11.1, 5.6 Hz, 2H), 1.59-1.49 (m, 4H), 1.53 (s, 18H), 1.28 (s, 18H), 1.32-1.13 (m, 20H), 0.95 (d, J=13.2 Hz, 2H), 0.89 (q, J=6.9 Hz, 8H), 0.35 (d, J=13.2 Hz, 2H). ¹³C NMR (126 MHz, c₆d₆) δ 157.60, 152.65, 149.33, 147.89, 143.13, 142.56, 139.54, 139.51, 129.91, 128.72, 128.59, 128.15, 127.04, 126.86, 125.16, 124.58, 124.26, 122.86, 122.35, 120.71, 117.65, 116.29, 115.69, 115.64, 115.53, 112.52, 108.73, 78.78, 77.39, 68.17, 34.61, 34.43, 32.06, 31.84, 31.72, 29.82, 29.28, 29.07, 27.22, 25.90, 22.72, 14.02.

Examples 39 and 40: synthesis of Precatalyst 5: a precatalyst of formula (II) wherein R¹, R², and R⁵ to R⁹ are H, R³ and R⁴ are each triethylmethyl, each R¹⁰ is tertiary-butyl, M is Zr, each X is benzyl, and subscript n is 2; and Precatalyst 6: a precatalyst of formula (I/) wherein R¹, R², and R⁵ to R⁹ are H, R³ and R⁴ are each triethylmethyl, each R¹⁰ is tertiary-butyl, M is Hf, each X is benzyl, and subscript n is 2

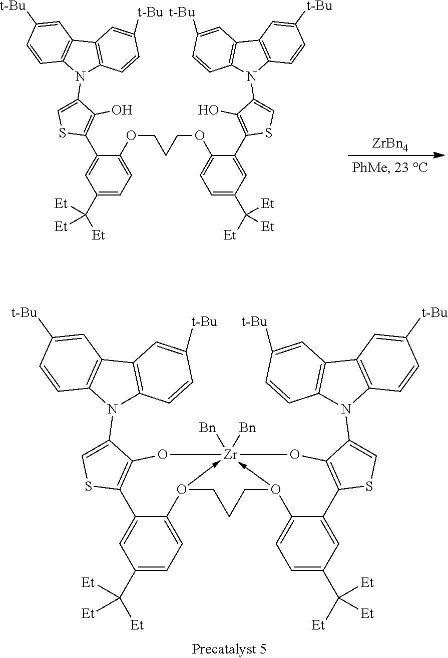

The thiophene ligand was azeotropically dried using PhMe (4×10 mL) prior to use. To a clear colorless solution of the thiophene (59.7 mg, 0.05078 mmol, 1.00 eq) in PhMe (24.0 mL) in a continuous purge, nitrogen filled glovebox at 23° C. was added a solution of ZrBn₄ (26.6 mg, 0.05839 mmol, 1.15 eq) in PhMe (2.13 mL) in a dropwise manner. After stirring (500 rpm) for 15 mins the golden yellow solution was filtered using a 0.45 μm PTFE submicron filter connected to a 0.20 μm PTFE submicron filter, rinsed with PhMe (3×3 mL), and concentrated to afford the zirconium complex as a golden yellow solid (72.5 mg, 0.05020 mmol, 99%). NMR indicated product. ¹H NMR (500 MHz, c₆d₆) δ 8.54 (dd, J=2.0, 0.6 Hz, 2H), 8.12 (dd, J=1.9, 0.6 Hz, 2H), 7.58-7.54 (m, 4H), 7.43 (dd, J=8.7, 1.9 Hz, 2H), 7.36-7.32 (m, 2H), 7.22 (dd, J=8.7, 0.6 Hz, 2H), 7.11-7.05 (m, 4H), 6.91 (dd, J=8.6, 2.5 Hz, 2H), 6.87 (s, 2H), 6.86-6.83 (m, 2H), 6.31-6.28 (m, 4H), 5.19 (d, J=8.6 Hz, 2H), 3.98 (dt, J=11.3, 6.8 Hz, 2H), 3.10 (dt, J=11.2, 5.2 Hz, 2H), 1.61 (s, 18H), 1.55 (dt, J=14.5, 7.3 Hz, 6H), 1.46 (dq, J=14.5, 7.1 Hz, 6H), 1.24 (s, 18H), 0.99 (d, J=12.2 Hz, 2H), 0.83 (p, J=5.8 Hz, 2H), 0.57-0.47 (m, 4H), 0.52 (t, J=7.4 Hz, 18H).

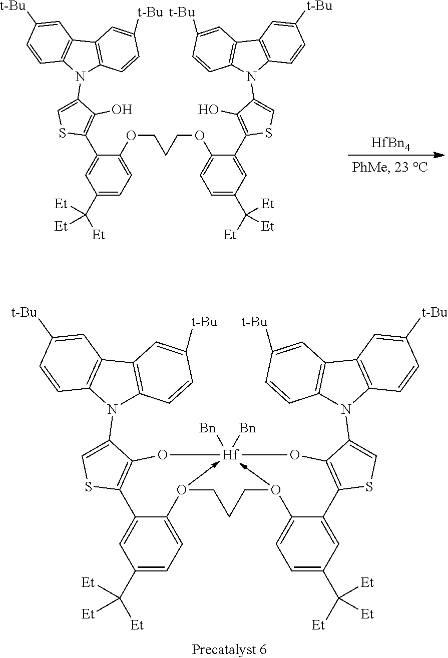

The thiophene ligand was azeotropically dried using PhMe (4×10 mL) prior to use. To a clear colorless solution of the thiophene (28.3 mg, 0.02407 mmol, 1.00 eq) in PhMe (18.0 mL) in a continuous purge, nitrogen filled glovebox at 23° C. was added a solution of HfBn₄ (15.7 mg, 0.02888 mmol, 1.20 eq) in PhMe (1.26 mL) in a dropwise manner. After stirring (500 rpm) for 15 mins the golden yellow solution was filtered using a 0.45 μm PTFE submicron filter connected to a 0.20 μm PTFE submicron filter, rinsed with PhMe (3×3 mL), and concentrated to afford the hafnium complex as a pale yellow solid (35.2 mg, 0.02294 mmol, 95%). NMR indicated product. ¹H NMR (500 MHz, c₆d₆) δ 8.55 (dd, J=2.0, 0.6 Hz, 2H), 8.12 (dd, J=1.9, 0.6 Hz, 2H), 7.58-7.54 (m, 4H), 7.41 (dd, J=8.7, 1.9 Hz, 2H), 7.33 (dd, J=8.5, 0.6 Hz, 2H), 7.14-7.08 (m, 6H), 6.94 (dd, J=8.7, 2.5 Hz, 2H), 6.87 (s, 2H), 6.84-6.80 (m, 2H), 6.31-6.28 (m, 4H), 5.18 (d, J=8.6 Hz, 2H), 4.01 (dt, J=11.4, 6.9 Hz, 2H), 3.11 (dt, J=11.4, 5.3 Hz, 2H), 1.61 (s, 18H), 1.56 (dt, J=14.7, 7.3 Hz, 6H), 1.50-1.43 (m, 6H), 1.24 (s, 18H), 0.89-0.79 (m, 2H), 0.83 (d, J=13.3, 2H), 0.57-0.49 (m, 2H), 0.52 (t, J=7.4 Hz, 18H), 0.20 (d, J=13.1 Hz, 2H). ¹³C NMR (126 MHz, c₆d₆) δ 153.81, 152.50, 147.39, 146.28, 142.81, 142.47, 139.42, 139.33, 129.91, 129.25, 128.94, 128.85, 128.59, 128.40, 127.10, 127.07, 126.72, 125.28, 124.60, 122.80, 122.57, 122.20, 120.73, 117.26, 116.29, 116.20, 115.65, 112.38, 110.01, 108.76, 78.16, 76.46, 43.57, 34.66, 34.36, 32.08, 31.67, 28.73, 26.94, 7.77.

Examples 41 and 42: synthesis of Precatalyst 7: a precatalyst of formula (II) wherein R¹, R², and R⁵ to R⁹ are H, R³ and R⁴ are each 3,5-di-(t-butyl)phenyl, each R¹⁰ is tertiary-butyl, M is Zr, each X is benzyl, and subscript n is 2; and Precatalyst 8: a precatalyst of formula (II) wherein R¹, R², and R⁵ to R⁹ are H, R³ and R⁴ are each 3,5-di-(t-butyl)phenyl, each R¹⁰ is tertiary-butyl, M is Hf, each X is benzyl, and subscript n is 2.

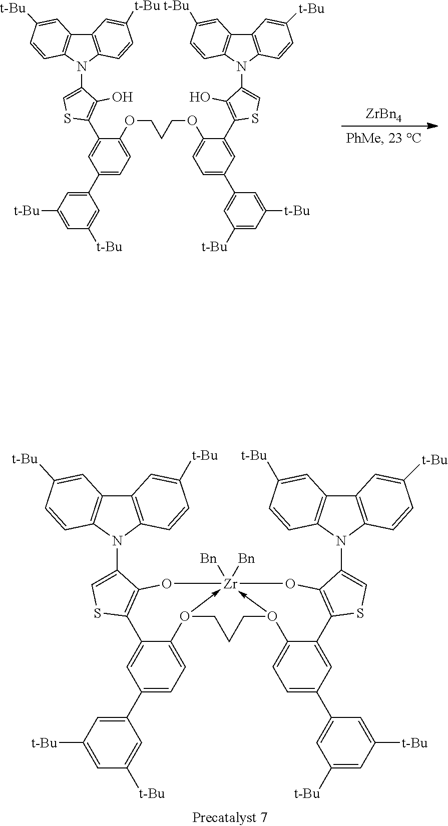

The thiophene ligand was azeotropically dried using PhMe (4×10 mL) prior to use. To a clear golden brown solution of the thiophene (46.4 mg, 0.03422 mmol, 1.00 eq) in PhMe (18.6 mL) in a continuous purge, nitrogen filled glovebox at 23° C. was added a solution of ZrBn₄ (17.9 mg, 0.03935 mmol, 1.15 eq) in PhMe (1.43 mL) in a dropwise manner. After stirring (500 rpm) for 15 mins the golden yellow solution was filtered using a 0.45 μm PTFE submicron filter connected to a 0.20 μm PTFE submicron filter, rinsed with PhMe (3×3 mL), and concentrated to afford the zirconium complex as a golden yellow foam (55.4 mg, 0.03404 mmol, 99%). NMR indicated product. ¹H NMR (400 MHz, toluene) 6 8.51 (d, J=1.9 Hz, 2H), 8.24-8.20 (m, 2H), 7.79 (d, J=2.3 Hz, 2H), 7.59-7.52 (m, 1 OH), 7.33 (d, J=8.5 Hz, 2H), 7.28 (ddd, J=8.4, 4.1, 1.5 Hz, 4H), 7.00-6.95 (m, 4H), 6.91 (s, 2H), 6.77 (t, J=7.3 Hz, 2H), 6.31-6.27 (m, 4H), 5.06 (d, J=8.4 Hz, 2H), 4.04 (dt, J=11.3, 6.7 Hz, 2H), 3.15 (dt, J=10.9, 5.3 Hz, 2H), 1.64 (s, 18H), 1.39 (s, 36H), 1.28 (s, 18H), 1.00 (p, J=6.0 Hz, 2H), 0.88 (d, J=13.4 Hz, 2H), 0.31 (d, J=13.4 Hz, 2H). ¹³C NMR (101 MHz, toluene) δ 155.79, 152.62, 151.24, 146.46, 142.96, 142.46, 141.27, 139.77, 139.57, 139.52, 137.34, 136.85, 136.77, 129.21, 127.05, 125.20, 124.16, 123.01, 122.50, 121.83, 120.72, 117.78, 116.26, 115.62, 112.33, 108.68, 76.33, 73.81, 34.76, 34.73, 34.40, 32.21, 31.66, 31.28, 27.48.

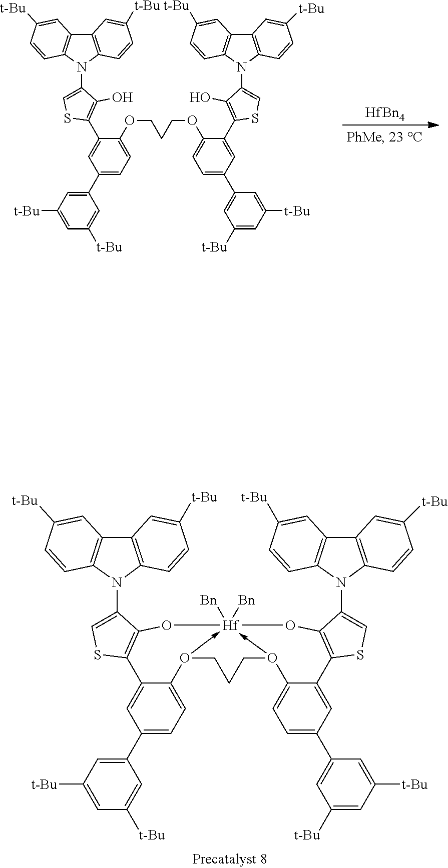

The thiophene ligand was azeotropically dried using PhMe (4×10 mL) prior to use. To a clear golden brown solution of the thiophene (37.0 mg, 0.02729 mmol, 1.00 eq) in PhMe (13.6 mL) in a continuous purge, nitrogen filled glovebox at 23° C. was added a solution of HfBn₄ (17.1 mg, 0.03138 mmol, 1.15 eq) in PhMe (1.37 mL) in a dropwise manner. After stirring (500 rpm) for 15 mins the pale golden yellow solution was filtered using a 0.45 μm PTFE submicron filter connected to a 0.20 μm PTFE submicron filter, rinsed with PhMe (3×3 mL), and concentrated to afford the hafnium complex as a pale golden yellow foam (44.8 mg, 0.02613 mmol, 96%). NMR indicated product. ¹H NMR (400 MHz, toluene) 6 8.51 (d, J=1.9 Hz, 2H), 8.24-8.21 (m, 2H), 7.79 (d, J=2.3 Hz, 2H), 7.59-7.52 (m, 1OH), 7.33 (d, J=8.5 Hz, 2H), 7.28 (ddd, J=8.4, 4.1, 1.5 Hz, 4H), 6.99-6.96 (m, 4H), 6.91 (s, 2H), 6.77 (t, J=7.3 Hz, 2H), 6.32-6.26 (m, 4H), 5.06 (d, J=8.4 Hz, 2H), 4.04 (dt, J=11.3, 6.7 Hz, 2H), 3.15 (dt, J=10.9, 5.3 Hz, 2H), 1.64 (s, 18H), 1.39 (s, 36H), 1.28 (s, 18H), 0.99 (q, J=6.0 Hz, 2H), 0.88 (d, J=13.4 Hz, 2H), 0.31 (d, J=13.4 Hz, 2H). ¹³C NMR (101 MHz, toluene) δ 155.15, 152.67, 151.27, 147.74, 143.05, 142.47, 141.70, 139.75, 139.51, 137.35, 136.85, 136.77, 129.34, 126.70, 125.20, 124.16, 122.99, 122.42, 121.86, 121.65, 120.60, 117.80, 116.28, 115.56, 112.48, 108.63, 78.94, 76.82, 34.76, 34.73, 34.39, 32.21, 31.65, 31.28, 27.25.

Examples 43 and 44: synthesis of Precatalyst 9: a precatalyst of formula (II) wherein R¹, R², and R⁵ to R⁹ are H, R³ and R⁴ are each CH₃(CH₂)₇,(CH₃)₂Si— (octyl-dimethyl-silyl), each R¹⁰ is tertiary-butyl, M is Zr, each X is benzyl, and subscript n is 2; and Precatalyst 10: a precatalyst of formula (II) wherein R¹, R², and R⁵ to R⁹ are H, R³ and R⁴ are each CH₃(CH₂)₇,(CH₃)₂Si— (octyl-dimethyl-silyl), each R¹⁰ is tertiary-butyl, M is Hf, each X is benzyl, and subscript n is 2.

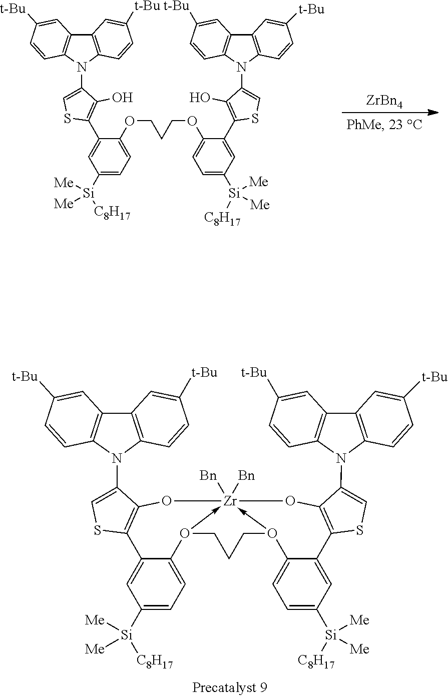

The thiophene ligand was azeotropically dried using PhMe (4×10 mL) prior to use. To a clear colorless solution of the thiophene (39.4 mg, 0.02985 mmol, 1.00 eq) in PhMe (16.7 mL) in a continuous purge, nitrogen filled glovebox at 23° C. was added a solution of ZrBn₄ (15.6 mg, 0.03432 mmol, 1.15 eq) in PhMe (1.30 mL) in a dropwise manner. After stirring (500 rpm) for 15 mins the pale golden yellow solution was filtered using a 0.45 μm PTFE submicron filter connected to a 0.20 μm PTFE submicron filter, rinsed with PhMe (3×3 mL), and concentrated to afford the zirconium complex as a pale golden yellow foam (47.4 mg, 0.02978 mmol, 99%). NMR indicated product. ¹H NMR (400 MHz, C₆D6) δ 8.60 (d, J=1.9 Hz, 2H), 8.20 (d, J=1.8 Hz, 2H), 7.78 (d, J=1.6 Hz, 2H), 7.59 (dd, J=8.6, 1.9 Hz, 2H), 7.53 (dd, J=8.7, 1.9 Hz, 2H), 7.35 (d, J=8.5 Hz, 2H), 7.30 (d, J=8.6 Hz, 2H), 7.22 (dd, J=8.0, 1.7 Hz, 2H), 7.06 (t, J=7.7 Hz, 4H), 6.91 (s, 2H), 6.82 (tt, J=7.3, 1.3 Hz, 2H), 6.21-6.16 (m, 4H), 5.14 (d, J=8.0 Hz, 2H), 3.97 (dt, J=11.1, 6.7 Hz, 2H), 3.07 (dt, J=10.9, 5.2 Hz, 2H), 1.66 (s, 18H), 1.37-1.27 (m, 24H), 1.26 (s, 18H), 1.06 (d, J=12.3 Hz, 2H), 0.96-0.91 (m, 6H), 0.87 (p, J=5.7 Hz, 2H), 0.71 (t, J=7.8 Hz, 4H), 0.58 (d, J=12.3 Hz, 2H), 0.22 (s, 6H), 0.22 (s, 6H). ¹³C NMR (101 MHz, C₆D₆) δ 157.26, 152.55, 146.69, 143.00, 142.59, 139.53, 139.44, 138.52, 136.14, 135.09, 130.59, 128.96, 128.36, 128.31, 127.01, 126.89, 126.54, 125.15, 124.59, 122.93, 122.41, 122.39, 120.77, 117.50, 116.42, 116.34, 115.73, 112.33, 108.82, 76.46, 74.87, 34.74, 34.39, 33.70, 32.17, 32.02, 31.67, 29.85, 29.43, 29.35, 27.47, 23.91, 22.77, 15.60, 14.05, −3.15, −3.34.

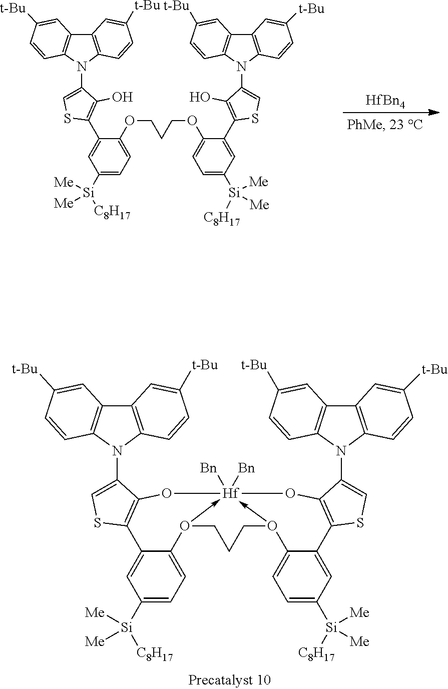

The thiophene ligand was azeotropically dried using PhMe (4×10 mL) prior to use. To a clear colorless solution of the thiophene (41.0 mg, 0.03106 mmol, 1.00 eq) in PhMe (16.4 mL) in a continuous purge, nitrogen filled glovebox at 23° C. was added a solution of HfBn₄ (19.4 mg, 0.03572 mmol, 1.15 eq) in PhMe (1.55 mL) in a dropwise manner. After stirring (500 rpm) for 15 mins the pale golden yellow solution was filtered using a 0.45 μm PTFE submicron filter connected to a 0.20 am PTFE submicron filter, rinsed with PhMe (3×3 mL), and concentrated to afford the hafnium complex as an off-white foam (51.2 mg, 0.03050 mmol, 98%). NMR indicated product. ¹H NMR (400 MHz, CD₆) δ 8.64-8.60 (m, 2H), 8.21 (dd, J=1.9, 0.7 Hz, 2H), 7.78 (d, J=1.6 Hz, 2H), 7.58 (dd, J=8.6, 1.9 Hz, 2H), 7.52 (dd, J=8.8, 1.9 Hz, 2H), 7.36-7.32 (m, 2H), 7.25 (dd, J=8.0, 1.7 Hz, 2H), 7.20 (dd, J=8.7, 0.6 Hz, 2H), 7.14-7.07 (m, 4H), 6.90 (s, 2H), 6.80 (tt, J=7.3, 1.2 Hz, 2H), 6.21-6.16 (m, 4H), 5.14 (d, J=8.0 Hz, 2H), 3.97 (dt, J=11.2, 6.6 Hz, 2H), 3.06 (dt, J=11.0, 5.4 Hz, 2H), 1.67 (s, 18H), 1.37-1.27 (m, 24H), 1.26 (s, 18H), 0.97-0.84 (m, 1OH), 0.72 (t, J=7.8 Hz, 4H), 0.30-0.25 (m, 2H), 0.23 (s, 6H), 0.22 (s, 6H). ¹³C NMR (101 MHz, CD₆) δ 156.77, 152.62, 147.59, 143.06, 142.59, 139.48, 139.43, 138.92, 136.21, 135.06, 128.96, 128.73, 128.31, 128.19, 127.11, 127.05, 126.70, 125.32, 125.25, 124.65, 122.89, 122.64, 122.31, 120.74, 117.54, 116.31, 115.78, 115.65, 112.41, 108.78, 78.88, 76.68, 34.74, 34.39, 33.71, 32.17, 32.03, 31.67, 29.85, 29.44, 29.35, 27.23, 23.91, 22.78, 15.58, 14.05, −3.15, −3.35.

Examples 45 and 46: synthesis of Precatalyst 11: a precatalyst of formula (II) wherein R¹, R², and R⁵ to R⁹ are H, R³ and R⁴ are each 4-(triethylmethyl)phenyl (4-Et₃C-Ph), each R¹⁰ is tertiary-butyl, M is Zr, each X is benzyl, and subscript n is 2; and Precatalyst 12: a precatalyst of formula (II) wherein R¹, R², and R⁵ to R⁹ are H, R³ and R⁴ are each 4-(triethylmethyl)phenyl (4-Et₃C-Ph), each R¹⁰ is tertiary-butyl, M is Hf, each X is benzyl, and subscript n is 2.

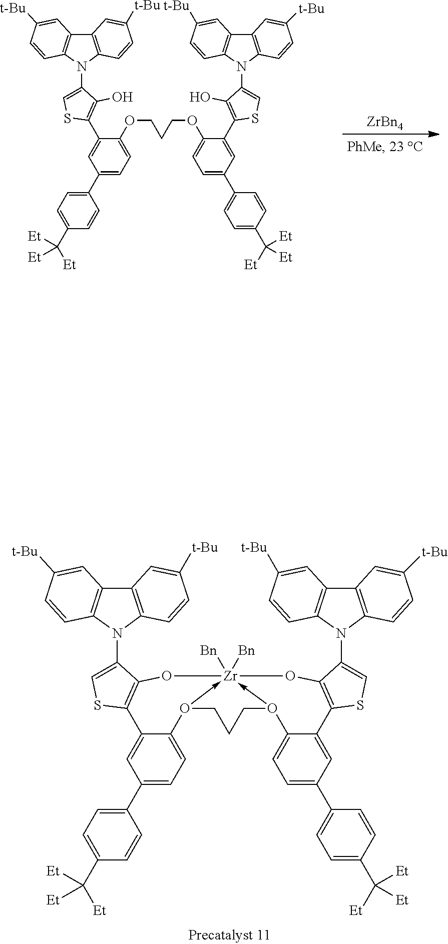

The thiophene ligand was azeotropically dried using PhMe (4×10 mL) prior to use. To a clear colorless solution of the thiophene (50.1 mg, 0.03773 mmol, 1.00 eq) in PhMe (20.4 mL) in a continuous purge, nitrogen filled glovebox at 23° C. was added a solution of ZrBn₄ (19.8 mg, 0.04339 mmol, 1.15 eq) in PhMe (1.59 mL) in a dropwise manner. After stirring (500 rpm) for 15 mins the pale golden yellow solution was filtered using a 0.45 μm PTFE submicron filter connected to a 0.20 μm PTFE submicron filter, rinsed with PhMe (3×3 mL), and concentrated to afford the zirconium complex as a pale golden brown foam (59.3 mg, 0.03708 mmol, 98%). NMR indicated product. ¹H NMR (500 MHz, c₆d₆) δ 8.47 (d, J=2.0 Hz, 2H), 8.30 (d, J=1.8 Hz, 2H), 7.74 (d, J=2.3 Hz, 2H), 7.60-7.56 (m, 4H), 7.53-7.49 (m, 4H), 7.44-7.41 (m, 2H), 7.35 (d, J=8.4 Hz, 2H), 7.30-7.26 (m, 4H), 7.17 (dd, J=8.4, 2.4 Hz, 2H), 7.03 (t, J=7.7 Hz, 4H), 6.90 (d, J=0.5 Hz, 2H), 6.75 (dd, J=8.0, 6.7 Hz, 2H), 6.32-6.28 (m, 4H), 5.14 (d, J=8.3 Hz, 2H), 3.96 (dt, J=11.2, 6.6 Hz, 2H), 3.05 (dt, J=10.8, 5.2 Hz, 2H), 1.60 (d, J=7.3 Hz, 12H), 1.55 (s, 18H), 1.30 (s, 18H), 1.03 (d, J=12.1 Hz, 2H), 0.86 (p, J=5.8 Hz, 2H), 0.66 (d, J=12.1 Hz, 2H), 0.64 (t, J=7.4 Hz, 18H).

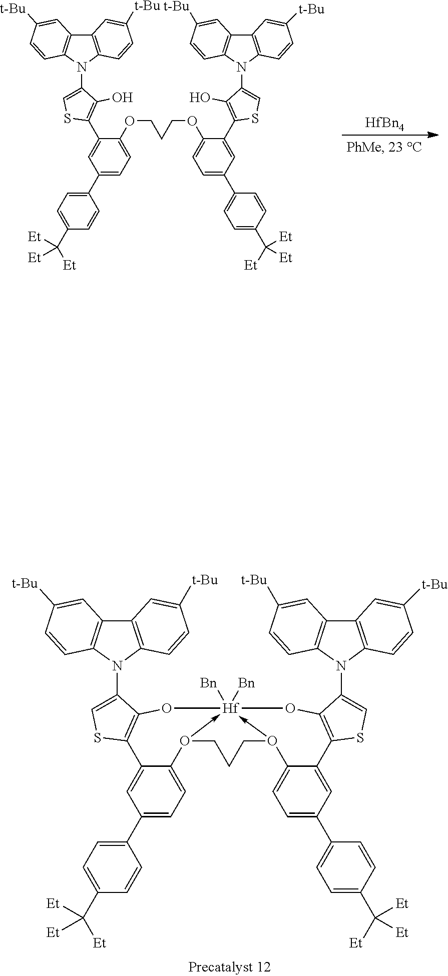

The thiophene ligand was azeotropically dried using PhMe (4×10 mL) prior to use. To a clear golden brown solution of the thiophene (30.9 mg, 0.02327 mmol, 1.00 eq) in PhMe (15.0 mL) in a continuous purge, nitrogen filled glovebox at 23° C. was added a solution of HfBn₄ (14.6 mg, 0.02676 mmol, 1.15 eq) in PhMe (1.17 mL) in a dropwise manner. After stirring (500 rpm) for 15 mins the pale golden yellow solution was filtered using a 0.45 μm PTFE submicron filter connected to a 0.20 μm PTFE submicron filter, rinsed with PhMe (3×3 mL), and concentrated to afford the hafnium complex as a pale golden yellow foam (38.8 mg, 0.02304 mmol, 99%). NMR indicated product. ¹H NMR (500 MHz, c₆d₆) δ 8.49 (dd, J=2.0, 0.6 Hz, 2H), 8.30 (dd, J=1.9, 0.6 Hz, 2H), 7.76 (d, J=2.4 Hz, 2H), 7.58-7.55 (m, 4H), 7.53-7.49 (m, 4H), 7.34 (ddd, J=8.5, 4.7, 0.6 Hz, 4H), 7.30-7.26 (m, 4H), 7.19 (dd, J=8.4, 2.4 Hz, 2H), 7.10-7.06 (m, 4H), 6.88 (s, 2H), 6.74 (tt, J=7.3, 1.3 Hz, 2H), 6.35-6.31 (m, 4H), 5.12 (d, J=8.4 Hz, 2H), 3.92 (dt, J=11.2, 6.5 Hz, 2H), 3.02 (dt, J=11.0, 5.4 Hz, 2H), 1.59 (q, J=7.4 Hz, 12H), 1.55 (s, 18H), 1.29 (s, 18H), 0.96 (d, J=13.3 Hz, 2H), 0.86 (p, J=5.9 Hz, 2H), 0.65 (t, J=7.4 Hz, 18H), 0.37 (d, J=13.3 Hz, 2H). ¹³C NMR (126 MHz, c₆d₆) δ 154.99, 152.69, 147.70, 146.95, 143.18, 142.62, 140.22, 139.53 (d, J=1.8 Hz), 136.81, 129.91, 129.23, 128.94, 128.64, 128.59, 128.18, 127.12, 126.71, 126.68, 125.30, 125.16, 124.62, 124.37, 123.56, 122.88, 122.43, 120.80, 117.77, 116.54, 115.69, 115.57, 112.50, 108.65, 78.92, 76.86, 43.49, 34.65, 34.46, 32.12, 31.76, 28.65, 27.28, 7.84.

Examples 47: synthesis of Precatalyst 13: a precatalyst of formula (II) wherein R¹, R², and R⁵ to R⁹ are H, R³ and R⁴ are each CH₃(CH₂)₇O— (octyloxy), each R¹⁰ is tertiary-butyl, M is Zr, each X is chloro, and subscript n is 2.

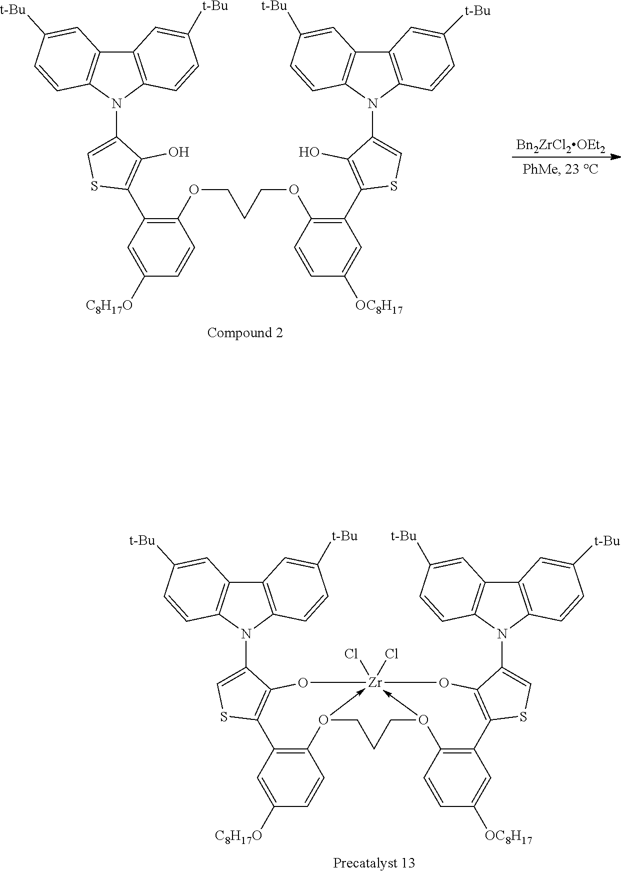

The thiophene ligand was azeotropically dried using PhMe (4×10 mL) prior to use. To a clear colorless solution of the thiophene (53.6 mg, 0.04337 mmol, 1.00 eq) in PhMe (20.1 mL) in a continuous purge, nitrogen filled glovebox at 23° C. was added a solution of ZrBn₂Cl₂ (20.0 mg, 0.04771 mmol, 1.10 eq) in PhMe (1.60 mL) in a dropwise manner. After stirring (500 rpm) for 15 mins the clear pale yellow solution was filtered using a 0.45 μm PTFE submicron filter connected to a 0.20 μm PTFE submicron filter, rinsed with PhMe (3×3 mL), and concentrated to afford the zirconium complex as an off-white foam (59.5 mg, 0.04263 mmol, 98%). NMR indicated product. ¹H NMR (400 MHz, C₆D6) δ 8.55 (d, J=2.0 Hz, 2H), 8.38 (s, 2H), 7.56-7.45 (m, 6H), 7.33 (t, J=7.9 Hz, 2H), 6.89 (s, 2H), 6.87 (d, J=3.0 Hz, 2H), 6.45 (d, J=9.0 Hz, 2H), 5.48-5.40 (m, 2H), 3.93-3.84 (m, 2H), 3.46 (ddt, J=23.9, 9.0, 6.5 Hz, 4H), 3.26-3.15 (m, 2H), 1.52 (s, 18H), 1.33 (s, 18H), 1.32-1.15 (m, 30H), 0.93 (t, J=7.0 Hz, 6H). ¹³C NMR (101 MHz, C₆D₆) δ 157.93, 152.73, 143.23, 142.69, 139.75 (d, J=5.9 Hz), 128.96, 128.55, 128.19, 127.16, 125.49, 124.94, 124.72, 122.59, 122.32, 118.20, 117.20, 116.40, 115.67, 115.28, 114.96, 112.05, 108.62, 79.26, 67.99, 34.57, 34.44, 32.03, 31.84, 31.67, 29.32, 29.28, 29.09, 28.04, 25.92, 22.72, 14.01.

### Examples 48 to 60: Spray-Drying Precatalysts to Make Spray-Dried Supported Catalyst Systems

Supported catalyst systems described in TABLE 3 above were made and spray-dried in a nitrogen-purged glove box. In an oven-dried jar, Cabosil™ TS-610 fumed silica was slurried in toluene until well dispersed, then a 10% solution by weight of MAO in toluene was added. The mixture was stirred magnetically 15 minutes, then the metal-ligand complex was added to the resulting slurry, and the mixture was stirred for 30 to 60 minutes. The mixture was spray-dried using a Buchi Mini Spray Dryer B-290 with the following parameters to yield the dried sample: Set Temperature: 140° C., Outlet Temperature: 75° C. (min.), aspirator setting of 95 rotations per minute (rpm), and pump speed of 150 rpm. TABLE 4 contains the amounts of the metal-ligand complex, fumed silica, 10% MAO solution, and toluene used to make each of the spray-dried supported catalyst systems 1 to 13. Quantities of reagents used are listed below in TABLE 4.

TABLE 4

| | | | Precatalyst | Fumed | 10% MAO | |
|---|---|---|---|---|---|---|
| | | | Weight, | Silica, | solution, | Toluene, |
| | Spray- | | Actual | Actual | Actual | Actual |
| Ex. | dried | Precatalyst | [expected] | [expected] | [expected] | [expected] |
| No. | SCS No. | No. | (g) | (g) | (g) | (g) |
| 48 | SCS 1 | 1 | 0.081 | 0.657 | 5.64 | 37.5 |
| 49 | SCS 2 | 2 | 0.083 | 0.633 | 5.47 | 37.5 |
| 50 | SCS 3 | 3 | 0.064 | 0.510 | 4.38 | 37.5 |
| 51 | SCS 4 | 4 | 0.071 | 0.530 | 4.59 | 37.5 |
| 52a | SCS 5a | 5 | 0.073 | 0.608 | 5.20 | 37.5 |
| 52b | SCS 5b | 5 | 0.049 | 0.91 | 7.33 | 37.5 |
| 53 | SCS 6 | 6 | 0.062 | 0.483 | 4.17 | 37.5 |
| 54 | SCS 7 | 7 | 0.062 | 0.452 | 3.93 | 37.5 |
| 55 | SCS 8 | 8 | 0.085 | 0.585 | 5.11 | 37.5 |
| 56 | SCS 9 | 9 | 0.038 | 0.640 | 5.17 | 37.5 |
| 57 | SCS 10 | 10 | 0.068 | 0.478 | 4.18 | 37.5 |
| 58 | SCS 11 | 11 | 0.064 | 0.475 | 4.13 | 37.5 |
| 59 | SCS 12 | 12 | 0.065 | 0.454 | 3.98 | 37.5 |
| 60 | SCS 13 | 13 | 0.017 | 0.687 | 5.40 | 37.5 |

### Gas-Phase Polymerizations Making Ethylene/i-Hexene Copolymers.

The spray dried catalysts described in TABLE 4 were used to catalyze gas phase polymerizations of ethylene monomer and 1-hexene comonomer to give ethylene/1-hexene copolymers (also called poly(ethylene-co-1-hexene) copolymers). The gas phase polymerizations conducted in a 2 liter (L), semi-batch, stainless steel autoclave gas phase polymerization reactor equipped with a mechanical agitator. For each polymerization run, the reactor was first dried (“baked out”) for 1 hour by charging the reactor with 200 grams (g) of NaCl, and heating the reactor contents at 100 00 under dry nitrogen for 30 minutes. Then 5 g of spray-dried methylaluminoxane/fumed silica (“SDMAO”) was added to the reactor under nitrogen pressure to scavenge any remaining water. The reactor was sealed, and the contents were stirred. The polymerizations were initiated by charging the reactor with hydrogen (H₂) and 1-hexene (C₆), then pressurizing the reactor with ethylene (C₂). The feed ratios of hydrogen, 1-hexene, and ethylene were set to achieve predetermined H₂/C₂ molar ratio and C₆/C₂ molar ratio in the reactor. Once the reactor reached a steady state, the supported catalyst system was charged into the reactor at 80° C. to start polymerization. The reactor temperature was brought to a predetermined polymerization temperature, typically 90° C. or 100° C. is used for these experiments, but any temperature from 750 to 115° C. may be used, and maintained at this polymerization temperature while keeping the ethylene, 1-hexene, and hydrogen feed ratios consistent for 1 hour. At the end of the 1-hour run, the feeds of hydrogen, 1-hexene, and ethylene were stopped, the reactor was cooled down, vented and opened. The resulting product mixture was washed with water and methanol, then dried to give the ethylene/1-hexene copolymer. The weight of the copolymer was recorded. Catalyst productivity (grams copolymer/gram catalyst-hour) and catalyst efficiency (grams copolymer/gram catalyst metal (Zr or Hf)) were determined to compare the amount of copolymer produced, based on ethylene and hexene uptake/consumption, relative to the amount of supported catalyst system added to the reactor. The copolymer samples were characterized by DSC and melt flow. The polymerization run conditions and results are listed in the following TABLES.

TABLE 5
Batch reactor conditions: Temp. = 100° C., C6/C2 (molar ratio) = 0.00X,
H2/C2 (molar ratio) = 0.00X, C2PP = 220 psi, run time = 1 hr, catalyst injection temp. = 80° C.

| | | | | Charge | Yield | Productivity | Efficiency | | C6 |
|---|---|---|---|---|---|---|---|---|---|
| Run | C6/C2 | H2/C2 | Catalyst | (mg) | (g) | (gPE/gCat/hr) | (gPE/gM) | I5, I21 | Uptake |
| 1 | 0.004 | 0.002 | SCS 1 | 3.1 | 35.0 | 13,500 | 3,536,200 | NF, 6.9 | 4.5% |
| 2 | 0.004 | 0.001 | SCS 1 | 3.7 | 39.7 | 7,400 | 1,923,000 | 0.1, 4.1 | 5.4% |
| 3 | 0.002 | 0.001 | SCS 1 | 3.1 | 21.1 | 5,400 | 1,418,700 | No | 2.8% |
| | | | | | | | | Flow | |
| 4 | 0.001 | 0.004 | SCS 1 | 5.0 | 39.9 | 7,300 | 1,897,000 | No | 1.5% |
| | | | | | | | | Flow | |
| 5 | 0.002 | 0.004 | SCS 1 | 5.2 | 32.1 | 5,800 | 1,523,800 | No | 2.4% |
| | | | | | | | | Flow | |
| 6 | 0.004 | 0.002 | SCS 2 | 10.7 | 27.4 | 2,500 | 333,500 | No | 7.5% |
| | | | | | | | | Flow | |
| 7 | 0.001 | 0.004 | SCS 3 | 5.2 | 126.9 | 24,300 | 6,338,200 | No | 0.9% |
| | | | | | | | | Flow | |
| 8 | 0.002 | 0.004 | SCS 3 | 4.8 | 117.4 | 27,300 | 7,120,700 | No | 1.8% |
| | | | | | | | | Flow | |
| 9 | 0.004 | 0.007 | SCS 3 | 5.6 | 81.5 | 14,300 | 3,732,500 | No | 4.0% |
| | | | | | | | | Flow | |
| 10 | 0.004 | 0.004 | SCS 3 | 5.0 | 85.7 | 16,900 | 4,411,100 | No | 4.1% |
| | | | | | | | | Flow | |
| 11 | 0.003 | 0.004 | SCS 3 | 5.3 | 65.1 | 12,000 | 3,132,100 | No | 2.8% |
| | | | | | | | | Flow | |
| 12 | 0.003 | 0.002 | SCS 3 | 5.1 | 50.5 | 9,400 | 2,453,500 | No | 2.7% |
| | | | | | | | | Flow | |
| 13 | 0.002 | 0.004 | SCS 4 | 4.7 | 19.8 | 3,500 | 466,900 | No | 2.3% |
| | | | | | | | | Flow | |
| 14 | 0.001 | 0.004 | SCS 4 | 5.0 | 18.9 | 3,100 | 413,500 | No | 2.1% |
| | | | | | | | | Flow | |
| 15 | 0.001 | 0.004 | SCS 5a | 5.3 | 83.9 | 15,400 | 4,016,500 | No | 1.4% |
| | | | | | | | | Flow | |
| 16 | 0.002 | 0.004 | SCS 5a | 5.8 | 145.4 | 24,100 | 6,291,200 | No | 2.2% |
| | | | | | | | | Flow | |
| 17 | 0.004 | 0.007 | SCS 5a | 6.4 | 19.9 | 17,500 | 4,567,700 | No | 5.0% |
| | | | | | | | | Flow | |
| 18 | 0.004 | 0.002 | SCS 5a | 6.0 | 23.4 | 20,000 | 5,220,200 | No | 5.1% |
| | | | | | | | | Flow | |
| 19 | 0.004 | 0.002 | SCS 5b | 12.3 | 36.3 | 5,500 | 3,010,600 | No | 5.2% |
| | | | | | | | | Flow | |
| 20 | 0.001 | 0.004 | SCS 5b | 5.7 | 20.8 | 4,400 | 2,408,500 | No | 1.9% |
| | | | | | | | | Flow | |
| 21 | 0.001 | 0.004 | SCS 6 | 5.6 | 59.5 | 10,100 | 1,347,200 | No | 1.2% |
| | | | | | | | | Flow | |
| 22 | 0.002 | 0.004 | SCS 6 | 5.5 | 45.5 | 7,700 | 1,027,100 | No | 1.3% |
| | | | | | | | | Flow | |
| 23 | 0.001 | 0.004 | SCS 7 | 5.4 | 158.7 | 31,900 | 8,313,500 | No | 1.6% |
| | | | | | | | | Flow | |
| 24 | 0.002 | 0.004 | SCS 7 | 5.3 | 26.5 | 16,000 | 4,175,900 | No | 2.3% |
| | | | | | | | | Flow | |
| 25 | 0.001 | 0.004 | SCS 8 | 5.1 | 22.1 | 4,100 | 546,900 | No | 1.5% |
| | | | | | | | | Flow | |
| 26 | 0.002 | 0.004 | SCS 8 | 5.4 | 21.7 | 3,500 | 466,900 | No | 3.1% |
| | | | | | | | | Flow | |
| 27 | 0.002 | 0.004 | SCS 9 | 10.1 | 28.3 | 2,500 | 1,370,300 | No | 1.9% |
| | | | | | | | | Flow | |
| 28 | 0.002 | 0.002 | SCS 9 | 10.6 | 62.7 | 5,100 | 2,795,400 | No | 1.5% |
| | | | | | | | | Flow | |
| 29 | 0.003 | 0.002 | SCS 9 | 10.5 | 53.3 | 4,400 | 2,411,800 | NF, 3.0 | 2.2% |
| 30 | 0.004 | 0.1 | SCS 10 | 5.7 | 7.2 | 2,500 | 333,500 | 0.3, 2.5 | 4.7% |
| 31 | 0.001 | 0.004 | SCS 11 | 5.4 | 120.6 | 21,700 | 5,664,700 | No | 1.7% |
| | | | | | | | | Flow | |
| 32 | 0.002 | 0.004 | SCS 11 | 5.1 | 97.9 | 18,400 | 4,810,700 | No | 3.5% |
| | | | | | | | | Flow | |
| 33 | 0.003 | 0.004 | SCS 11 | 5.1 | 50.0 | 10,100 | 2,636,200 | No | 5.5% |
| | | | | | | | | Flow | |
| 34 | 0.004 | 0.007 | SCS 11 | 5.6 | 43.7 | 7,400 | 1,919,700 | NF, 3.8 | 7.6% |
| 35 | 0.004 | 0.002 | SCS 11 | 5.6 | 25.6 | 11,500 | 3,001,100 | NF, 2.1 | 6.0% |
| 36 | 0.003 | 0.002 | SCS 11 | 5.0 | 46.8 | 9,300 | 2,427,400 | No | 3.9% |
| | | | | | | | | Flow | |
| 37 | 0.001 | 0.004 | SCS 12 | 5.0 | 4.1 | 2,100 | 280,100 | No | 1.1% |
| | | | | | | | | Flow | |
| 38 | 0.002 | 0.004 | SCS 12 | 5.3 | 10.8 | 1,800 | 240,100 | No | 2.4% |
| | | | | | | | | Flow | |
| 39 | 0.004 | 0.004 | SCS 13 | 5.0 | 20.5 | 6,600 | 7,235,300 | No | 3.4% |
| | | | | | | | | Flow | |
| 40 | 0.004 | 0.002 | SCS 13 | 5.2 | 19.3 | 8,500 | 9,318,100 | No | 3.5% |
| | | | | | | | | Flow | |
| 41 | 0.004 | 0.002 | SCS 13 | 3.7 | 18.9 | 7,100 | 7,783,400 | No | 4.2% |
| | | | | | | | | Flow | |
| 42 | 0.002 | 0.002 | SCS 13 | 3.6 | 24.7 | 6,800 | 7,454,500 | No | 2.1% |
| | | | | | | | | Flow | |

NF = No Flow.

TABLE 6
Batch reactor conditions: Temp. = 100° C., C6/C2 (molar ratio) = 0.00X,
H2/C2 (molar ratio) = 0.00X, C2PP = 220 psi, run time = 1 hr, catalyst injection temp. = 80° C.

| | | | | Mw | PDI | Mz | C6 | T_{M} | LCBf |
|---|---|---|---|---|---|---|---|---|---|
| Run | C6/C2 | H2/C2 | Catalyst | (g/mol) | (Mw/Mn) | (g/mol) | wt % | (° C.) | (LCB/1000 C) |
| 1 | 0.004 | 0.002 | SCS 1 | 206,100 | 5.1 | 2,228,100 | 6.6 | 114.7 | 0.105 |
| 2 | 0.004 | 0.001 | SCS 1 | 157,700 | 4.5 | 2,463,500 | 7.9 | 114.0 | 0.074 |
| 3 | 0.002 | 0.001 | SCS 1 | 228,200 | 4.6 | 2,965,500 | 5.1 | 118.3 | 0.073 |
| 4 | 0.001 | 0.004 | SCS 1 | 357,800 | 4.8 | 2,846,100 | 4.6 | 120.9 | 0.034 |
| 5 | 0.002 | 0.004 | SCS 1 | 248,400 | 4.2 | 1,962,300 | 5.3 | 118.6 | 0.045 |
| 6 | 0.004 | 0.002 | SCS 2 | 916,300 | 5.0 | 3,379,100 | 7.2 | 109.3 | N.D. |
| 7 | 0.001 | 0.004 | SCS 3 | 585,600 | 6.4 | 3,788,400 | 3.8 | 122.9 | N.D. |
| 8 | 0.002 | 0.004 | SCS 3 | 486,700 | 6.2 | 3,238,200 | 5.0 | 119.1 | 0.065 |
| 9 | 0.004 | 0.007 | SCS 3 | 384,200 | 5.7 | 2,869,900 | 6.9 | 111.3 | 0.040 |
| 10 | 0.004 | 0.004 | SCS 3 | 367,800 | 5.5 | 2,711,300 | 7.0 | 112.0 | 0.052 |
| 11 | 0.003 | 0.004 | SCS 3 | 444,400 | 5.7 | 3,171,500 | 5.7 | 115.2 | 0.043 |
| 12 | 0.003 | 0.002 | SCS 3 | 451,900 | 5.5 | 3,217,600 | 5.4 | 114.7 | 0.042 |
| 13 | 0.002 | 0.004 | SCS 4 | 1,162,900 | 5.9 | 4,013,600 | 5.4 | 122.5 | N.D. |
| 14 | 0.001 | 0.004 | SCS 4 | 1,186,900 | 5.7 | 3,981,900 | 3.8 | 122.9 | N.D. |
| 15 | 0.001 | 0.004 | SCS 5a | 367,400 | 3.7 | 2,198,400 | 3.6 | 123.6 | 0.026 |
| 16 | 0.002 | 0.004 | SCS 5a | 303,300 | 3.7 | 1,869,500 | 4.8 | 119.9 | 0.027 |
| 17 | 0.004 | 0.007 | SCS 5a | 198,900 | 4.3 | 1,282,800 | 8.2 | 112.2 | 0.058 |
| 18 | 0.004 | 0.002 | SCS 5a | 201,100 | 4.4 | 1,590,000 | 8.2 | 112.3 | 0.056 |
| 19 | 0.004 | 0.002 | SCS 5b | 219,100 | 4.8 | 2063,600 | 8.4 | 111.9 | N.D. |
| 20 | 0.001 | 0.004 | SCS 5b | 332,900 | 3.9 | 2,133,200 | 4.1 | 121.4 | N.D. |
| 21 | 0.001 | 0.004 | SCS 6 | 1,263,300 | 2.8 | 3,074,100 | 3.0 | 124.4 | N.D. |
| 22 | 0.002 | 0.004 | SCS 6 | N.D. | N.D. | N.D. | N.D. | 119.4 | N.D. |
| 23 | 0.001 | 0.004 | SCS 7 | 408,100 | 4.4 | 1,805,100 | 5.0 | 119.8 | 0.101 |
| 24 | 0.002 | 0.004 | SCS 7 | 418,800 | 5.9 | 1,926,500 | 6.3 | 119.4 | 0.261 |
| 25 | 0.001 | 0.004 | SCS 8 | 875,900 | 9.7 | 2,504,400 | 4.3 | 119.5 | N.D. |
| 26 | 0.002 | 0.004 | SCS 8 | N.D. | N.D. | N.D. | N.D. | 114.8 | N.D. |
| 27 | 0.002 | 0.004 | SCS 9 | 394,000 | 7.0 | 3,619,700 | 5.5 | 122.1 | 0.016 |
| 28 | 0.002 | 0.002 | SCS 9 | 410,500 | 10.4 | 3,801,000 | 4.0 | 126.00 | 0.078 |
| 29 | 0.003 | 0.002 | SCS 9 | 324,200 | 9.0 | 3,648,300 | 5.0 | 123.0 | 0.076 |
| 30 | 0.004 | 0.1 | SCS 10 | 185,500 | 3.4 | 979,000 | 8.6 | 120.2 | N.D. |
| 31 | 0.001 | 0.004 | SCS 11 | 283,500 | 4.9 | 2,254,700 | 6.1 | 119.2 | 0.126 |
| 32 | 0.002 | 0.004 | SCS 11 | N.D. | N.D. | N.D. | N.D. | 115.0 | N.D. |
| 33 | 0.003 | 0.004 | SCS 11 | 183,400 | 5.0 | 1,498,800 | 8.9 | 110.6 | 0.204 |
| 34 | 0.004 | 0.007 | SCS 11 | 153,300 | 5.6 | 1,530,500 | 11.0 | 107.9 | 0.164 |
| 35 | 0.004 | 0.002 | SCS 11 | 177,400 | 5.7 | 1,644,300 | 10.2 | 109.3 | 0.156 |
| 36 | 0.003 | 0.002 | SCS 11 | 202,300 | 6.2 | 1,911,600 | 8.4 | 111.9 | 0.135 |
| 37 | 0.001 | 0.004 | SCS 12 | 756,000 | 5.0 | 2,163,700 | 4.1 | 122.1 | N.D. |
| 38 | 0.002 | 0.004 | SCS 12 | 674,400 | 3.9 | 2,033,000 | 5.1 | 117.8 | N.D. |
| 39 | 0.004 | 0.004 | SCS 13 | 438,200 | 9.7 | 2,407,000 | 6.2 | 118.9 | 0.006 |
| 40 | 0.004 | 0.002 | SCS 13 | 409,300 | 9.7 | 2,254,000 | 6.7 | 118.3 | N.D. |
| 41 | 0.004 | 0.002 | SCS 13 | 419,300 | 10.5 | 2,397,900 | 7.2 | 117.9 | 0.007 |
| 42 | 0.002 | 0.002 | SCS 13 | 554,900 | 10.2 | 2,618,000 | 4.6 | 120.0 | 0.006 |

LCBf = amount of long-chain branches/1000 carbons.

In TABLES 5 and 6, the semi-batch reactor results for the effective spray-dried catalysts, SCS 1 to SCS-13 with differentiated substituents R³ and R⁴, are shown. Under process relevant high density conditions, the productivities and/or efficiencies for the inventive SCS 1, SCS 3, SCS 5, SCS 5a, SCS 6, SCS 7, SCS 9, SCS 11, and SCS 13 are high, with productivities up to 31,900 gPE/gCat/hr and efficiencies up to 9.3 MM gPE/gM under these conditions. Optimal productivity and efficiency were observed for spray-dried zirconium catalysts possessing substituents R³ and R⁴ such as, but not limited to, triethylmethyl, 3,5-di-t-butylphenyl, or octyloxy groups. Based on the melt flow data (12, 15, 121) as well as data from GPC analysis (Table 6), under these commercially relevant high density process conditions, the inventive catalysts can produce polyethylene copolymers with medium-to-ultra-high weight average molecular weight (Mw up to 1,300,000 g/mol), high Mz (up to 3,981,900 g/mol), broad molecular weight distribution (MWD) or polydispersity index (PDI), up to 10, broad Mw/Mz, up to 10.8, and high comonomer incorporation (up to 11 wt %). These Mw and Mz may be lowered by increasing the temperature as well as the H₂/C₂ and/or C₆/C₂ ratio used in the reactor. Also, based on comonomer consumption in the reactor, and/or T_{M} of the polymer, along with the GPC analysis, this data is indicative that each of these inventive spray-dried catalysts incorporate increased amounts of comonomer (1-hexene) under industrially relevant high density process conditions. This combination of high Mw and/or high Mz capability, broader MWD as well as Mz/Mw with higher comonomer incorporation makes a resin with potential advantageous properties.

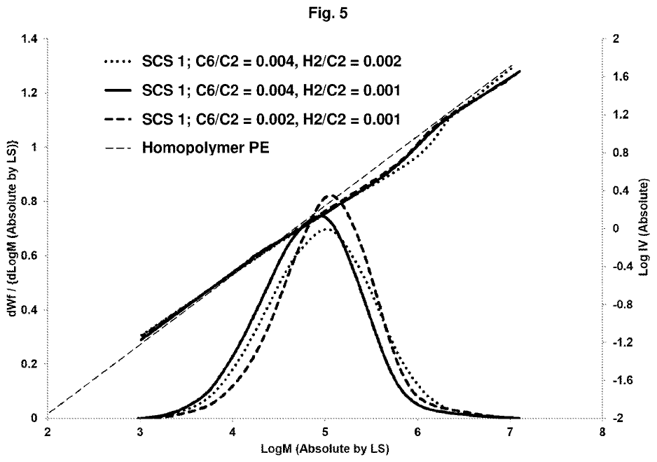

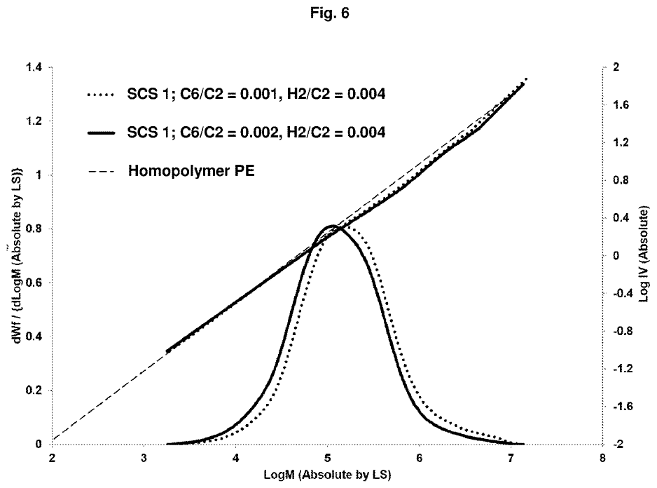

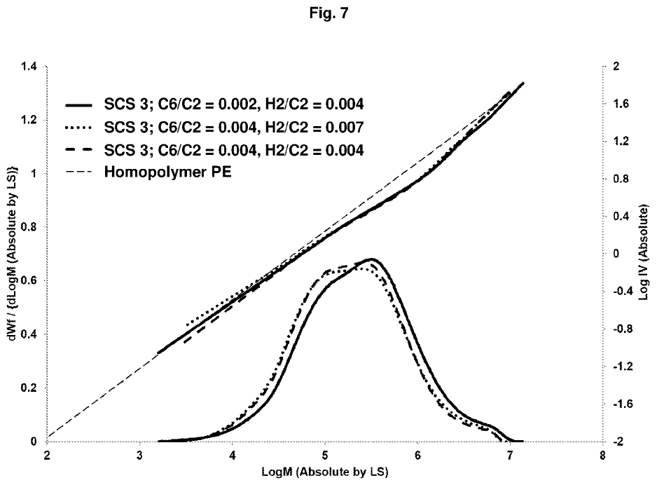

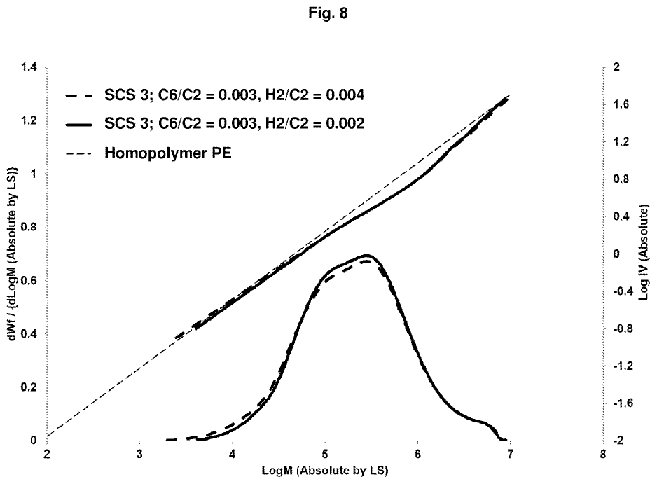

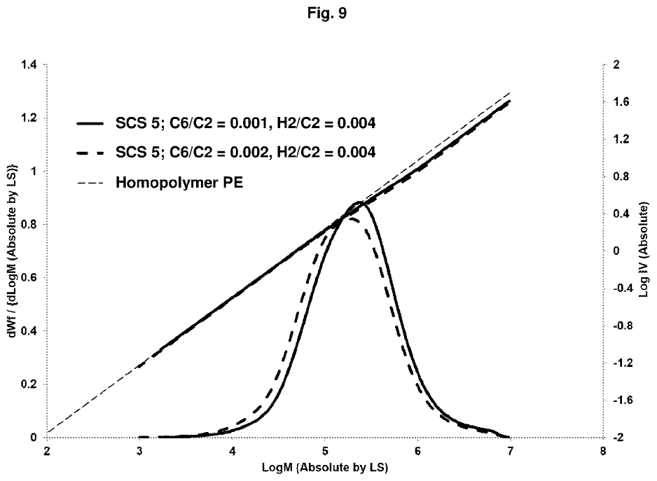

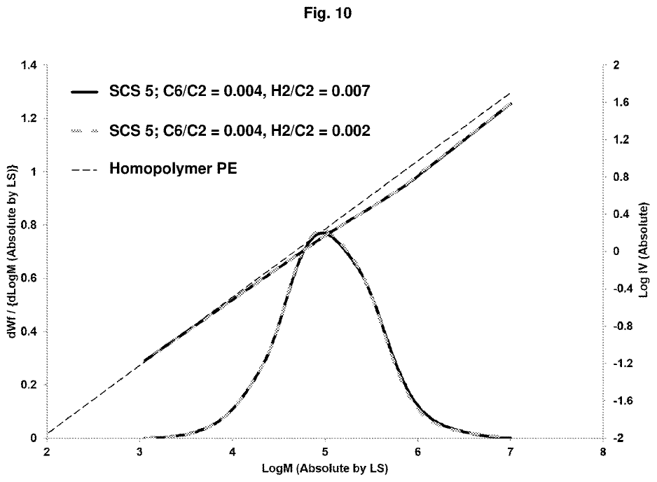

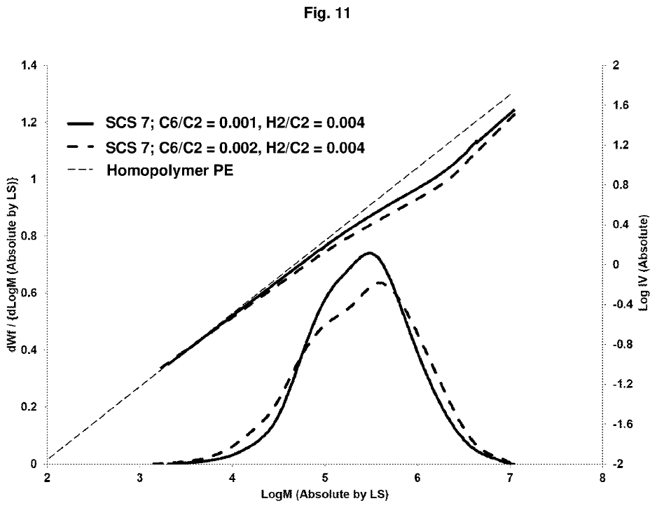

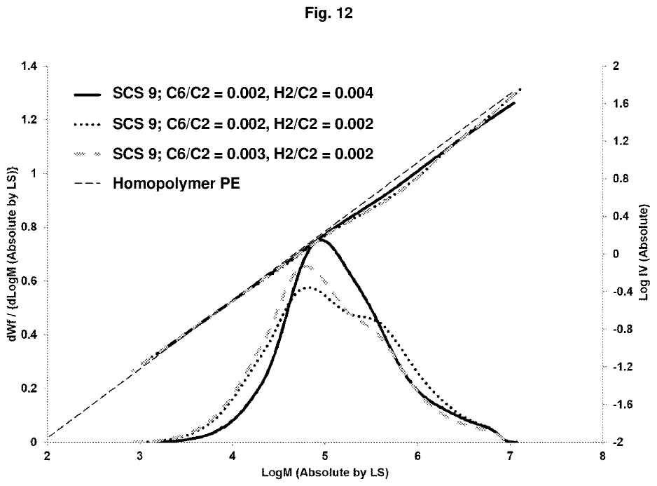

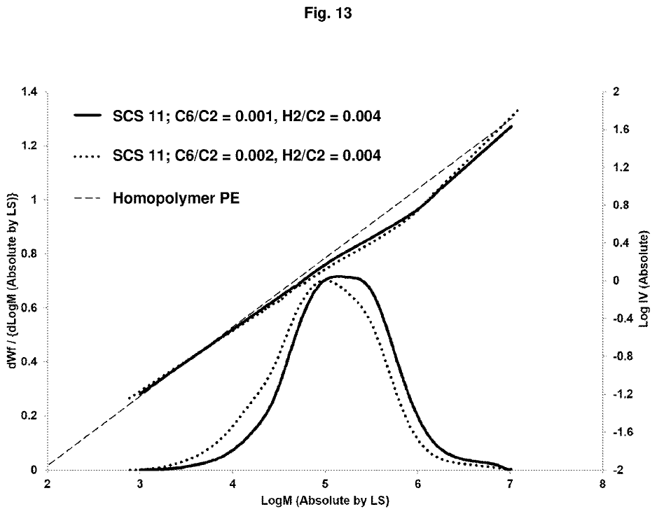

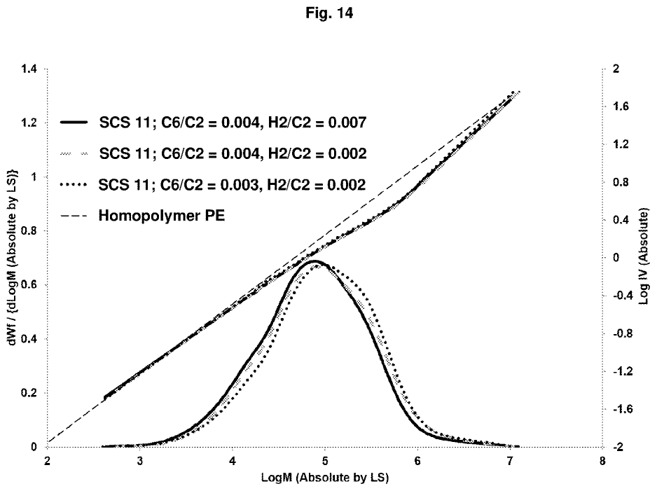

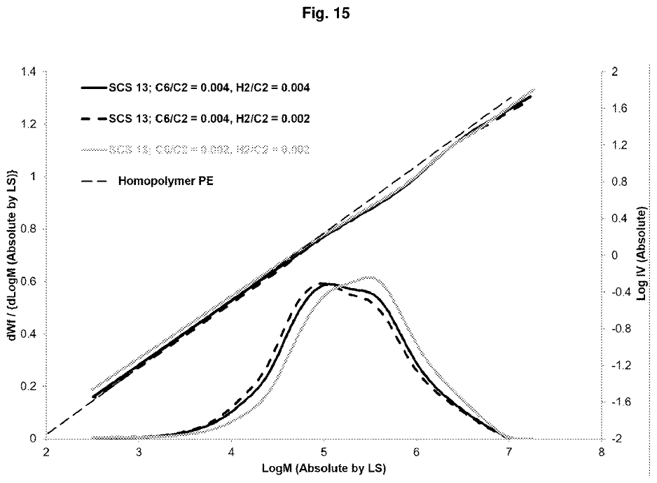

TABLE 6 also has data for the estimated LCBf of the polymers produced from the gas phase batch reactor experiments. LCBf is a measurement, or quantification, of the number of long-chain branches per 1000 carbon atoms (LCB/1000C) based on analysis of Mark-Houwink plots as shown in FIGS. 5 to 15, where the higher the LCBf, the higher the amount of long-chain branching. A model fit for LCBf was calculated using Microsoft Solver using Equation 1 below to fit a model curve to the experimental data, where the Log(IV) is calculated at a given log(Mw) (a) of a sample with an LCBf (b), weight percent comonomer (c) and number of carbons in comonomer (d). Equation 1 (Eq. 1): Log(IV)=LOG((0.000381478441772065*(10\hat{ }a)\hat{ }0.732)*((6/(b*(10\hat{ }a)/14,000)*(0.5*SQRT ((2+(b*(10\hat{ }a)/14,000))/(b*(10\hat{ }a)/14,000))*LN((SQRT(2+(b*(10\hat{ }a)/14,000))+SQRT((b*(10\hat{ }a)/14,000)))/(SQRT(2+(b*(10\hat{ }a)/14,000))−SQRT((b*(10\hat{ }a)/14,000))))−1))\hat{ }0.5)*(1−c/100*(d−2)/d)\hat{ }2). The inventive catalyst systems can produce polymers with a varied amount of long-chain branching (LCBf up to 0.261 long-chain branches/1000C). In particular, the inventive catalysts bearing a 3,5-di-t-butylphenyl or 4-triethylmethylphenyl substituent for R³ and R⁴ in Equation 1, SCS 7 and SCS 11, produce polymers with the highest degree of LCB for these inventive catalysts. This combination of high Mw and/or high Mz capability, broader MWD, high hexene incorporation combined with varying amounts of LCB also offers a resin with potential advantageous properties for both single and multi-catalyst applications.

### Slurry Phase Polymerization Experiments

Synthesis of Undried Supported Catalyst Systems for slurry phase polymerization. In a nitrogen filled continuous purge glovebox, a precatalyst of formula (II) is provided either in neat form, or as a solution thereof dissolved in toluene, or as a solid form wherein the precatalyst is already supported on spray-dried activator/hydrophobic fumed silica solids, wherein the activator is methylaluminoxane. This supported activator is called “SMAO” herein and is white in color. Unsupported precatalysts are diluted to 4.21 millimolar (mM) concentration in anhydrous deoxygenated toluene, and pipetted into oven-dried 4 mL or 8 mL scintillation vials containing a pre-weighed amount of the SMAO such that the resultant slurry has a catalyst formulation of 45 micromoles (pmol) Zr atom or Hf atom, as the case may be, per 1.0 grams (g) SMAO, unless otherwise noted. The slurry is stirred at 300 rotations per minute (rpm) and heated to 50° C. for 30 minutes, then returned to room temperature to give a slurry of an undried supported catalyst system (“ud-SCS”) in toluene. Colorization of the previously white SMAO indicates the precatalyst has been supported and activated. ¹H-NMR experiments of the slurry's liquid phase shows that there is no remaining precatalyst or unsupported active catalyst present in the liquid phase, which indicates the precatalyst has been fully converted to active catalyst in the ud-SCS. The slurries of ud-SCS at room temperature are vortexed and agitated at 700 rpm to produce a uniform dispersion thereof. The slurries of ud-SCS are agitated for at least one minute, and while vortexing is continued, aliquots are daughtered by positive displacement tip (PDT) into 8 mL vials. Daughtered aliquots of the ud-SCS will be diluted with isoparaffin solvent (Isopar E) to 50 millimoles (mmol) to 500 nmol per mL, depending on the expected catalytic activity. All catalyst materials and daughtered aliquots of ud-SCS are kept in a glovebox freezer at −30° C. until use in the slurry phase polymerization.

Preparation of slurry phase polymerization reactor cells in a drybox. A day prior to polymerization runs, 48 parallel pressure reactor (PPR) cells having module heads and module bodies were prepared as follows. Oven-dried, pre-weighed glass tubes were manually inserted into the reactor wells, polyether ether ketone (“PEEK”) stir paddles were attached to the module heads, and the module heads were attached to the module bodies. The reactor cells were heated to 190° C., purged with nitrogen for 10 hours, and cooled to 50° C. On the next day (day of the experiments), the reactors were purged twice with ethylene and vented completely to purge feed lines. The reactor cells were then pre-heated to 50° C. and the stir paddles turned on to a stirring rate of 800 rotations per minute (rpm), thereby giving prepared reactor cells.

Running slurry phase polymerizations in the prepared reactor cells in the drybox. The prepared reactor cells were partially filled (to an appropriate solvent level) with an isoparaffin hydrocarbon (“solvent”, Isopar-E from ExxonMobil), and olefin comonomer (for these experiments 1-hexene) using a robotic needle to later give a final total volume of 5 mL in each reactor cell (once all of the reagent solutions are added later). Following solvent injection, the reactor cells were heated to a target starting-the-polymerization temperature (in these experiments, 100° C.) and the stirring rate was increased. When the temperature of the reactor cells reached the starting—the polymerization temperature, which required about 10-30 minutes of heating, the reactor cells were pressurized to a target starting the polymerization pressure with either pure ethylene, or a gas mixture of ethylene and hydrogen from a gas accumulator, and until the solvent was saturated with the pure ethylene or the gas mixture, respectively, (as observed by the gas uptake). If the gas mixture of ethylene and hydrogen was used, once the solvent was saturated in all cells, the gas feed line was switched from the accumulator to pure ethylene for the remainder of the polymerization run.

Then a robotic synthesis protocol was initiated whereby activator (a slurry of SMAO in solvent) was injected first, followed by injection of a slurry of the conventionally-dried supported catalyst system. Both injections for a given reactor cell were completed before the robot started the injection into the next reactor cell in the sequence. Each reagent addition was chased with 500 microliters (μL) of the solvent to ensure all the reagent had been injected. After each reagent injection the needles were washed with the solvent both inside and outside the needle.

At the moment of the catalyst injection in each individual cell, a reaction timer and monitoring of each cell's pressure were started. The desired pressure (within approximately 2-6 psig) was maintained by adding supplemental amount of ethylene gas by opening the valve at the target pressure minus 2 psi and closing the valve when the pressure reached 2 psi above target pressure. All drops in reactor cell pressure were cumulatively recorded as uptake of ethylene for the duration of the run. The slurry phase polymerization reactions proceeded for 90 minutes or to an ethylene uptake of 90 psi, whichever occurred first, and then were quenched by adding a 60 psi overpressure of 10% (v/v) CO₂ in argon. Data collection of each cell continued for 5 minutes after the quench. After the last cell finished quenching, any potential gas leaks were identified from the cell pressure and ethylene uptake curves were noted. The reactors were cooled down to 50° C., vented, and the glass tubes were removed from the module bodies. The glass tubes were removed from the drybox and the volatiles then removed using a rotary evaporator. The glass tubes were re-weighed to obtain reaction yields, the obtained polyethylene homopolymer or ethylene/1-hexene copolymer products were analyzed by GPC using a High Throughput High Temperature Gel Permeation Chromatography (HT-HT-GPC) Test Method.

High Throughput High Temperature Gel Permeation Chromatography (HT-HT-GPC) Test Method was performed using a Robot Assisted Delivery (RAD) system equipped with a PolymerChar infrared detector (IR5) and Agilent PLgel Mixed A columns. Decane (10 μL) was added to each sample for use as an internal flow marker. Samples were first diluted in 1,2,4-trichlorobenzene (TCB) stabilized with 300 ppm butylated hydroxyl toluene (BHT) at a concentration of 10 mg/mL and dissolved by stirring at 160° C. for 120 minutes. Prior to injection samples were further diluted with TCB stabilized with BHT to a concentration of 2 mg/mL. Samples (250 μL) were eluted through one PL-gel 20 μm (50×7.5 mm) guard column followed by two PL-gel 20 μm (300×7.5 mm) Mixed-A columns maintained at 160° C. with TCB stabilized with BHT at a flowrate of 1.0 mL/min. The total run time was 24 minutes. To calibrate for molecular weight (MW), Agilent EasiCal polystyrene standards (PS-1 and PS-2) were analyzed to create a 3^{rd} order MW calibration curve. Molecular weight units were converted from polystyrene (PS) to polyethylene (PE) using a daily Q-factor calculated around 0.4 using the average of 5 reference samples of known MW.

The table below lists the slurry phase polymerization conditions: Temp.=100° C., IsoparE=5 mL, C₆/C₂ (molar ratio)=0.6/1 in liquid, H₂/C₂ (molar ratio)=0.0016/1.0 in liquid, run time=90 minutes maximum (5400 seconds), Quench time=time needed to uptake 90 psi of ethylene; the faster the quench time, the more active the catalyst is. All catalysts are 45 pmol Zr or Hf/1 g SMAO, except ud-SCS 3 which is 10 pmol Zr/g SMAO. N.D.=not determined. The loading of all catalysts in the slurry phase reactor was 25 nanomoles except ud-SCS 3, which was 10 nmol. The “ud-SCS” means undried supported catalyst system, which is prepared according to the procedure of Synthesis of Undried Supported Catalyst Systems for slurry phase polymerization.

TABLE 9
Slurry Parallel Pressure Reactor (PPR) conditions: Temp. =
X ° C., IsoparE = 5 mL, C6/C2 (molar ratio) = 0.6
in liquid, H2/C2 (molar ratio) = 0.0016 in liquid, run time = 90 mins max (5400 s).

| | | | | Quench | | | | | |
|---|---|---|---|---|---|---|---|---|---|
| | Temp. | Loading | Uptake | Time | Mw | PDI | Mz | C6 | Yield |
| Catalyst | (° C.) | (nmol) | (psi) | (s) | (g/mol) | (Mw/Mn) | (g/mol) | (wt %) | (mg) |
| SCS 14 | 80 | 25 | 91 | 143 | 69,600 | 7.2 | 797,000 | 9.6 | 175 |
| SCS 14 | 100 | 25 | 90 | 148 | 57,400 | 5.8 | 600,500 | 9.5 | 166 |
| SCS 15 | 80 | 25 | 90 | 282 | 117,800 | 5.5 | 349,200 | 8.1 | 142 |
| SCS 15 | 100 | 25 | 90 | 207 | 160,100 | 4.2 | 408,100 | 9.0 | 154 |
| SCS 16 | 80 | 10 | 90 | 1,103 | 72,500 | 4.5 | 244,500 | 2.8 | 120 |
| SCS 16 | 100 | 10 | 90 | 825 | 43,200 | 3.7 | 218,700 | 3.7 | 117 |

Under these process relevant high density conditions, high activity is deemed as quench times of 1,000 seconds or faster at catalyst charges of 25 nmol for a catalyst of 45 pmol Zr or Hf/g SMAO. The quench time is the time it takes to consume 90 psi of ethylene during the experiment, where the faster the time, the more active the catalyst. SCS 1, SCS 2, and SCS 3 each exhibit significantly high activity in slurry polymerization process as indicated by the low catalyst loading (10-25 nmol) combined with the fast quench time (143-1,103 s) in the slurry polymerization process. GPC analysis also indicates that these inventive supported catalysts can produce ethylene/hexene copolymers with high Mw (>100,000 g/mol under these conditions for PPR process), high Mz, copolymers with broad PDI (2 4.0 for PPR process) as well as Mz/Mw, and high 1-hexene incorporation (2 3.0 wt % under these conditions for slurry PPR process). This combination of high activity combined with the ability to make ethylene/hexene copolymers with high Mw and/or high Mz, broad PDI, broad Mz/Mw with higher alpha-olefin comonomer incorporation offers an ethylene/alpha-olefin copolymer resin with advantageous properties in an industrially relevant slurry polymerization process.

Claimed embodiments follow.

1. A substituted 2-hydroxythiophene compound of formula (I): 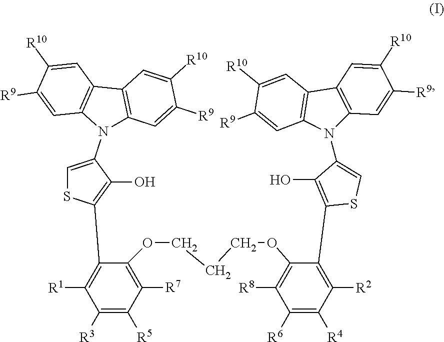
or a Group 1 or Group 2 metal salt thereof, wherein:
R¹ and R² independently are H or a halogen;
R³ and R⁴ independently are H, a halogen, a (C₁-C₂₀)hydrocarbyl, a (C₁-C₁₀)alkoxy, or a Si((C₁-C₁₀)alkyl)₃;
R⁵ and R⁶ independently are H or a halogen;
R⁷ and R⁸ independently are H or a halogen; and
each R⁹ is H and each R¹⁰ is a (C₁-C₂₀)hydrocarbyl; or each R¹⁰ is H and each R⁹ is a (C₁-C₂₀)hydrocarbyl.

2. A precatalyst of formula (II): 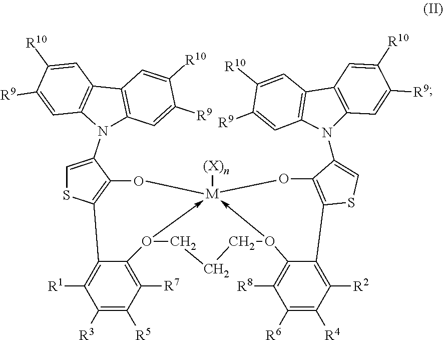
wherein:
M is Ti, Hf, or Zr;
subscript n is 1 or 2; and
each X independently is selected from a monodentate ligand independently chosen from a hydrogen atom, a (C₁-C₅₀)hydrocarbyl, a (C₁-C₅₀)heterohydrocarbyl, a (C₁-C₅₀)organoheteryl, a halogen atom, a dialkylamino, or a dialkyl carbamate;
R¹ and R² independently are H or a halogen;
R³ and R⁴ independently are H, a halogen, a (C₁-C₂₀)hydrocarbyl, a (C₁-C₁₀)alkoxy, or a Si((C₁-C₁₀)alkyl)₃;
R⁵ and R⁶ independently are H or a halogen;
R⁷ and R⁸ independently are H or a halogen; and
each R⁹ is H and each R¹⁰ is a (C₁-C₂₀)hydrocarbyl; or each R¹⁰ is H and each R⁹ is a (C₁-C₂₀)hydrocarbyl.

3. A supported catalyst system comprising the precatalyst of formula (II) of claim 2, a support material, and an activator.

4. A method of making the supported catalyst system of claim 3 comprising step (a) or comprising steps (b) and (c):
(a) spray drying a mixture of an inert hydrocarbon solvent, the precatalyst of formula (II), the support material, and the activator to make the supported catalyst system; or
(b) spray drying a mixture of an inert hydrocarbon solvent, the support material and the activator to make a spray-dried supported activator, and
(c) mixing the precatalyst of formula (II) with the spray-dried supported activator and an inert hydrocarbon solvent to make the supported catalyst system.

5. The invention of claim 1 wherein:
R¹ and R² are different, or R¹ and R² are identical; or R¹ and R² are H; or R¹ and R² are F; or
R³ and R⁴ are different, or R³ and R⁴ are identical, or R³ and R⁴ are a halogen, a (C₁-C₂₀)hydrocarbyl, a (C₁-C₁₀)alkoxy, or a Si((C₁-C₁₀)alkyl)₃, or R³ and R⁴ are a (C₁-C₁₀)alkyl or a (C₁-C₁₀)alkoxy; or
R⁵ and R⁶ are different, or R⁵ and R⁶ are identical, or R⁵ and R⁶ are H, or R⁵ and R⁶ are F; or
R⁷ and R⁸ are different, or R⁷ and R⁸ are identical, or R⁷ and R⁸ are H, or R⁷ and R⁸ are F; or
each R⁹ is H and each R¹⁰ is tertiary-butyl, 4-tert-butylphenyl, 4-triethylmethylphenyl, 3,5-dimethylphenyl, or 3,5-di-tert-butylphenyl; or each R¹⁰ is H and each R⁹ is 3,5-di-tert-butylphenyl; or
a combination of the foregoing definitions of R¹ to R¹⁰.

6. The invention of claim 2 wherein:
M is Hf or Zr; or
subscript n is 2; or
each X is benzyl or each X is Cl; or
M is Hf or Zr, subscript n is 2, and each X is benzyl; or
M is Hf or Zr, subscript n is 2, and each X is chloro.

7. The substituted 2-hydroxythiophene compound of formula (I) of claim 1 selected from the group consisting of compounds 1 to 7 in TABLE 1:
TABLE 1

| Cmpd | R¹/R² | R³/R⁴ | R⁵/R⁶ | R⁷/R⁸ | | |
|---|---|---|---|---|---|---|
| No. | each is | each is | each is | each is | R⁹ | R¹⁰ |
| 1 | H | t-Octyl | H | H | H | t-Bu |
| 2 | H | OctylO | H | H | H | t-Bu |
| 3 | H | Et₃C— | H | H | H | t-Bu |
| 4 | H | 3,5-dtBP | H | H | H | t-Bu |
| 5 | H | C₈Me₂Si | H | H | H | t-Bu |
| 6 | H | 4-Et₃CPh | H | H | H | t-Bu |

Wherein “Cmpd No.” is compound number, t-Bu is tertiary-butyl; t-Octyl is (CH₃)₃CCH₂C(CH₃)₂—; OctylO is CH₃(CH₂)₇O—; 35dtBP is 3,5-di-tert-butylphenyl or 3,5-di(t-Bu)-Ph; C₈Me₂Si is CH₃(CH₂)₇(CH₃)₂Si— (octyl-dimethyl-silyl); and 4Et₃CPh is 4-triethylmethylphenyl (i.e., 4-[(CH₃CH₂)₃C]—C₆H₄—).

8. The precatalyst of formula (II) of claim 2 selected from the group consisting of precatalyst numbers 1 to 13 in TABLE 2:
TABLE 2

| Precatalyst | Make from Formula | | X | |
|---|---|---|---|---|
| No. | (I) Compound No. | M | each is | n |
| 1 | 1 | Zr | Benzyl | 2 |
| 2 | 1 | Hf | Benzyl | 2 |
| 3 | 2 | Zr | Benzyl | 2 |
| 4 | 2 | Hf | Benzyl | 2 |
| 5 | 3 | Zr | Benzyl | 2 |
| 6 | 3 | Hf | Benzyl | 2 |
| 7 | 4 | Zr | Benzyl | 2 |
| 8 | 4 | Hf | Benzyl | 2 |
| 9 | 5 | Zr | Benzyl | 2 |
| 10 | 5 | Hf | Benzyl | 2 |
| 11 | 6 | Zr | Benzyl | 2 |
| 12 | 6 | Hf | Benzyl | 2 |
| 13 | 2 | Zr | Cl | 2 |

9. The supported catalyst system of claim 3 selected from the group consisting of spray-dried supported catalyst system numbers SCS 1 to SCS 13 and undried supported catalyst system numbers SCS 14 to SCS 16 in TABLE 3:
TABLE 3

| | Make from Formula | Support | | Catalyst Drying |
|---|---|---|---|---|
| SCS No. | (II) Precatalyst No. | Material | Activator | Method |
| SCS 1 | 1 | HPFS1 | MAO | Spray |
| SCS 2 | 2 | HPFS1 | MAO | Spray |
| SCS 3 | 3 | HPFS1 | MAO | Spray |
| SCS 4 | 4 | HPFS1 | MAO | Spray |
| SCS 5 | 5 | HPFS1 | MAO | Spray |
| SCS 6 | 6 | HPFS1 | MAO | Spray |
| SCS 7 | 7 | HPFS1 | MAO | Spray |
| SCS 8 | 8 | HPFS1 | MAO | Spray |
| SCS 9 | 9 | HPFS1 | MAO | Spray |
| SCS 10 | 10 | HPFS1 | MAO | Spray |
| SCS 11 | 11 | HPFS1 | MAO | Spray |
| SCS 12 | 12 | HPFS1 | MAO | Spray |
| SCS 13 | 13 | HPFS1 | MAO | Spray |
| | | | |
|---|---|---|---|
| SCS 14 | 1 | SMAO | None (undried) |
| SCS 15 | 2 | SMAO | None (undried) |
| SCS 16 | 3 | SMAO | None (undried) |

wherein “HPFS1” is a hydrophobic fumed silica made from untreated fumed silica and a hydrophobing agent that is dichlorodimethylsilane; and “MAO” is methylaluminoxane; and wherein “SMAO” is spray dried methylaluminoxane/HPFS1, wherein HPFS1 is made from untreated fumed silica and a hydrophobing agent that is dichlorodimethylsilane.

10. The supported catalyst system of claim 3 is that which has been shown to make by gas phase polymerization an ethylene/1-hexene copolymer having a weight-average molecular weight greater than 500,000 grams per mole and/or a z-average molecular weight greater than 2,000,000 grams per mole.

11. A method of making a polyolefin in a gas phase polymerization process, the method comprising contacting one or more olefin monomers with the supported catalyst system; of claim 3 in a gas phase polymerization reactor under gas phase polymerization conditions to make a polyolefin polymer.

12. The method of claim 11 having any one of limitations (i) to (v):
(i) wherein the one or more olefin monomers comprises ethylene or a combination of ethylene and propylene or a combination of ethylene and a (C₄-C₂₀)alpha-olefin and wherein the polyolefin polymer is an ethylene homopolymer or an ethylene/propylene copolymer or an ethylene/(C₄-C₂₀)alpha-olefin copolymer;
(ii) wherein the one or more olefin monomers comprises ethylene or a combination of ethylene and a (C₄-C₂₀)alpha-olefin and the polyolefin polymer is an ethylene homopolymer or an ethylene/(C₄-C₂₀)alpha-olefin copolymer; wherein the ethylene homopolymer or an ethylene/(C₄-C₂₀)alpha-olefin copolymer has a weight-average molecular weight 500,000 grams per mole or greater, or a z-average molecular weight of 2,000,000 grams per mole or greater, or both; or
(iii) wherein the one or more olefin monomers comprises a combination of ethylene and a (C₄-C₂₀)alpha-olefin and wherein the polyolefin polymer is an ethylene/(C₄-C₂₀)alpha-olefin copolymer having a polydispersity index (PDI) of a ratio of weight-average molecular weight to number-average molecular weight (Mw/Mn) of greater than or equal to 4.0, or a broad molecular weight distribution of a ratio of z-average molecular weight to weight-average molecular weight (Mz/Mw) greater than or equal to 3.5, or both;
(iv) any one of limitations (i) to (iii) wherein the (C₄-C₂₀)alpha-olefin is 1-hexene;
(v) a combination of limitations (ii) and (iii) or a combination of limitations (ii), (iii), and (iv).

13. A method of making a polyolefin in a gas phase polymerization process, the method comprising contacting one or more olefin monomers with the supported catalyst system, of claim 3 in a gas phase polymerization reactor under gas phase polymerization conditions to make a polyolefin polymer with long-chain branching.

14. A method of making a polyolefin in a slurry phase polymerization process, the method comprising contacting one or more olefin monomers with the supported catalyst system of claim 3 in a slurry phase polymerization reactor under slurry phase polymerization conditions to make a polyolefin polymer.

15. The method of claim 14 wherein the one or more olefin monomers comprises ethylene or a combination of ethylene and propylene or a combination of ethylene and a (C₄-C₂₀)alpha-olefin and the polyolefin polymer comprises an ethylene homopolymer or an ethylene/propylene copolymer or an ethylene/(C₄-C₂₀)alpha-olefin copolymer.
